SEQUENCE LISTING (101 sequences)

SEQ ID NO: 1 - DNA, 1685 bp, Pantoea sp.
  ribosomal RNA nucleic acid sequence
ccggacccctccccccgcaattccccctccactccccatttttctattctacttctctctttgttgtgtgctctcgttttatatttgttcacgggggggaaaaacgctaccgcggcgcgcgcttgttagagtttgatcatggctcagattgaactctgttggcaggcctaacacatgcaagtcgaacggcagcacagaagagcttgctctttgggtggcgagtggcggacgggtgagtaatgtctgtgaaactgcccgatggagggggataactactggaaacggtagctaataccgcataacgtcgcaagaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcggctcggccgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggattgtagtctgcaactcgactccatgaagtcggaatcgctagtaatcgtagatcagaatgctacggtgaatacgttcccggaccttgtacacaccgcccgtcacaccatgggagtgtattgcaaaaagaagtaggtagcttaaccttcgggaggacgcttaccactttgggattcatgacaagaactgaagtcctaacaagataaccgtaaagagtgtgtaagggtgatgggggacccgtacggggcggataagatcgca

SEQ ID NO: 2 - DNA, 1507 bp, Bacillus sp.
  ribosomal RNA nucleic acid sequence
gagtttgaatcatggctcaggacgaacgctggcggcgtgcctaatacatgcaagtcgagcggacagaagggagcttgctcccggatgttagcggcggacgggtgagtaacacgtgggtaacctgcctgtaagactgggataactccgggaaaccggagctaataccgcatagttccttgaaccgcatggttaaagaatgaaagacggtttcggctgtcacttacagatggacccgcggcgcattagctagttggtggggtaatggctcaccaaggcgacgatgcgtagccgacctgagagggcgaccgcccaaactgggactgagacacgaccaaaactaatacgggaggcagcagtagggaatcttccgcaatggacgaaagtctgacggagcaacgccgcgtgagtgatgaaggttttcggatcgtaaagctctgttgttagggaagaacaagtgcgagagtaactgctcgcaccttgacggtacctaaccagaaagccacggctaactacgtgccagcagccgcggtaatacgtaggtggcaagcgttgtccggaattattgggcgtaaagggctcgcaggcggtttcttaagtctgatgtgaaagcccccggctcaaccggggagggtcattggaaactgggaaacttgagtgcagaagaggagagtggaattccacgtgtagcggtgaaatgcgtagagatgtggaggaacaccagtggcgaaggcgactctctggtctgtaactgacgctgaggagcgaaagcgtggggagcgaacaggattagataccctggtagtccacgccgtaaacgatgagtgctaagtgttagggggtttccgccccttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaaccctagagatagggctttcccttcggggacagagtgacaggtggtgcatggttgtcgtcagctcgtgtcgtgagatgttgggttaagtcccgcaacgagcgcaacccttgatcttagttgccagcattcagttgggcactctaaggtgactgccggtgacaaaccggaggaaggtggggatgacgtcaaatcatcatgccccttatgacctgggctacacacgtgctacaatggacagaacaaagggctgcaagaccgcaaggtttagccaatcccataaatctgttctcagttcggatcgcagtctgcaactcgactgcgtgaagctggaatcgctagtaatcgcggatcaccatgccgcggtgaatacgttcccgggccttgtacacaccgcccgtcacaccacgagagtttgcaacacccgaagtcggtgaggtaacctttatggagccagccgccgaaggtggggcagatgattggggtgaagtcgtaacagggta

SEQ ID NO: 3 - DNA, 1503 bp, Pseudomonas sp.
  ribosomal RNA nucleic acid sequence
agagtttgaatcatggctcagattgaacgctggcggcaggcgtaacacatgcaagtcgagcggtagagagaagcttgcttctcttgagagcggcggacgggtgagtaatgcctaggaatctgcctggtagtgggggataacgttcggaaacggacgctaataccgcatacgtcctacgggagaaagcaggggaccttcgggccttgcgctatcagatgagccctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggttgtagattaatactctgcaattttgacgttaccgacagaataagcaccggctaactctgtgccagcagccgcggtaatacagagggtgcaagcgttaatcggaattactgggcgtaaagcgcgcgtaggtggtttgttaagttggatgtgaaatccccgggctcaacctgggaactgcattcaaaactgactgactagagtatggtagagggtggtggaatttcctgtgtagcggtgaaatgcgtagatataggaaggaacaccagtggcgaaggcgaccacctggactaatactgacactgaggtgcgaaagcgtggggagcaaacaggattagataccctggtagtccacgccgtaaacgatgtcaactaaccgttggaagcacttgagcttttagtggcgcagctaacgcagtaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtcttaaccttcgggaggacggttaccacggtgtgatttcatgactgggggtgaagtcgttaacaaggtag

SEQ ID NO: 4 - DNA, 1409 bp, Sphingobium sp.
  ribosomal RNA nucleic acid sequence
tgtggtcgcctgcctccttacggttagctcaacgccttcgagtgaatccaactcccatggtgtgacgggcggtgtgtacaaggcctgggaacgtattcaccgcggcatgctgatccgcgattactagcgattccgccttcacgctctcgagttgcagagaacgatccgaactgagacgacttttggagattagctccctctcgcgaggtggctgcccaccgtagtcgccattgtagcacgtgtgtagcccaacgcgtaagggccatgaggacttgacgtcatccccaccttcctccggcttatcaccggcggttcctttagagtacccaactaaatgctggcaactaaaggcgagggttgcgctcgttgcgggacttaacccaacatctcacgacacgagctgacgacagccatgcagcacctgtcacctatccagccgaactgaaggaaagtgtctccacgatccgcgatagggatgtcaaacgttggtaaggttctgcgcgttgcttcgaattaaaccacatgctccaccgcttgtgcaggcccccgtcaattcctttgagttttaatcttgcgaccgtactccccaggcggataacttaatgcgttagctgcgccaccaaaacaccatgtgccctgacagctagttatcatcgtttacggcgtggactaccagggtatctaatcctgtttgctccccacgctttcgcacctcagcgtcaataccagtccagtgagccgccttcgccactggtgttcttccgaatatctacgaatttcacctctacactcggaattccactcacctctcctggattcaagctatctagtttcaaaggcagttccggggttgagccccgggctttcacctctgacttgaatagccgcctacgtgcgctttacgcccagtaattccgaacaacgctagctccctccgtattaccgcggctgctggcacggagttagccggagcttattctcccggtactgtcattatcatcccgggtaaaagagctttacaaccctaaggccttcatcactcacgcggcattgctggatcaggctttcgcccattgtccaatattccctactgctgcctcccgtaggagtctgggccgtgtctcagtcccagtgtggctgatcatcctctcagaccagctaaggatcgtcgccttggtgagcctttacctcaccaactagctaatcctacgcgggctcatccttgggcgataaatctttggacttacgtcatcatccggtattagcttccgtttccagaagttattccgaacccaagggcagattcccacgcgttacgcacccgtgcgccactagatccgaagatctcgttcgacttgcatgtattaggcatgccgccagcgttcgttctgagccatgatccaaactct

SEQ ID NO: 5 - DNA, 1527 bp, Lysobacter sp.
  ribosomal RNA nucleic acid sequence
aagagtttgatcctggctcagagtgaacgctggcggcaggcctaacacatgcaagtcgaacggcagcacagaggagcttgctccttgggtggcgagtggcggacgggtgaggaatacgtcggaatctgcctatttgtgggggataacgtagggaaacttacgctaataccgcatacgacctacgggtgaaagcggaggaccttcgggcttcgcgcagatagatgagccgacgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccatgccgcgtgtgtgaagaaggccttcgggttgtaaagcacttttgtccggaaagaaaagccttgggttaataccctgaggtcatgacggtaccggaagaataagcaccggctaacttcgtgccagcagccgcggtaatacgaagggtgcaagcgttactcggaattactgggcgtaaagcgtgcgtaggtggtttgttaagtctgatgtgaaagccctgggctcaacctgggaatggcattggaaactggctgactagagtgcggtagagggtagtggaattcccggtgtagcagtgaaatgcgtagatatcgggaggaacatctgtggcgaaggcgactacctggaccagcactgacactgaggcacgaaagcgtggggagcaaacaggattagataccctggtagtccacgccctaaacgatgcgaactggatgttgggagcaacttggctctcagtatcgaagctaacgcgttaagttcgccgcctgggaagtacggtcgcaagactgaaactcaaaggaattgacgggggcccgcacaagcggtggagtatgtggtttaattcgatgcaacgcgaagaaccttacctggccttgacatccacggaactttccagagatggattggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacggccagggctacacacgtactacaatggtagggacagagggctgcaaacccgcgagggcaagccaatcccagaaaccctatctcagtccggatcggagtctgcaactcgactccgtgaagtcggaatcgctagtaatcgcagatcagcattgctgcggtgaatacgttcccgggccttgtacacaccgcccgtcacaccatgggagtttgttgcaccagaagcaggtagcttaaccttcgggagggcgcttgccacggtgtggccgatgactggggtgaagtcgtaacaaggtagccgtatcggaaggtgcggctgg

SEQ ID NO: 6 - DNA, 1522 bp, Pseudomonas sp.
  ribosomal RNA nucleic acid sequence
aagagtttgatcatggctcagattgaacgctggcggcaggcctaacacatgcaagtcgagcggcagcacgggtacttgtacctggtggcgagcggcggacgggtgagtaatgcctaggaatctgcctagtagtgggggataacgtccggaaacgggcgctaataccgcatacgtcctacgggagaaagtgggggatcttcggacctcacgctattagatgagcctaggtcggattagctagttggtgaggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagttacctaatacgtgattgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttctagagatagattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgcgaatcagaatgtcgcggtgaatacgttcccgggccttgtacacaccgcccgtcacaccatgggagtgggttgcaccagaagtagctagtctaaccttcgggaggacggttaccacggtgtgattcatgactggggtgaagtcgtaacaaggtagccgtaggggaacctgcggctgga

SEQ ID NO: 7 - DNA, 1339 bp, Gordonia sp.
  ribosomal RNA nucleic acid sequence
agagtttgatcctggctcaggacgaacgctggcggcgtgcttaacacatgcaagtcgaacggaaaggcccagcttgctgggtactcgagtggcgaacgggtgagtaacacgtgggtgatctgccctgcactctgggataagcctgggaaactgggtctaataccggatatgaccaactgtcgcatggtggttggtggaaagcttttgcggtgtgggatgggcccgcggcctatcagcttgttggtggggtaatggcctaccaaggcgacgacgggtagccgacctgagagggtgatcggccacactgggactgagacacggcccagactcctacgggaggcagcagtggggaatattgcacaatgggcgcaagcctgatgcagcgacgccgcgtgagggatgacggccttcgggttgtaaacctctttcaccagggacgaagcgtgagtgacggtacctggagaagaagcaccggccaactacgtgccagcagccgcggtaatacgtagggtgcgagcgttgtccggaattactgggcgtaaagagctcgtaggcggtttgtcgcgtcgtctgtgaaattctgcaactcaattgcaggcgtgcaggcgatacgggcagacttgagtactacaggggagactggaattcctggtgtagcggtgaaatgcgcagatatcaggaggaacaccggtggcgaaggcgggtctctgggtagtaactgacgctgaggagcgaaagcgtgggtagcgaacaggattagataccctggtagtccacgccgtaaacggtgggtactaggtgtgggttccttttcacgggatccgtgccgtagctaacgcattaagtaccccgcctggggagtacggccgcaaggctaaaactcaaaggaattgacgggggcccgcacaagcggcggagcatgtggattaattcgatgcaacgcgaagaaccttacctgggtttgacatacaccagacgcggctagagatagtcgttcccttgtggttggtgtacaggtggtgcatggctgtcgtcagctcgtgtcgtgagatgttgggttaagtcccgcaacgagcgcaacccttgtcctgtattgccagcgggttatgccggggacttgcaggagactgccggggtcaactcggaggaaggtggggatgacgtcaagtcatcatgccccttatgtccagggcttcacacatgctacaatggctggtacagagggctgcgataccgtgaggtggagcgaatcccttaaagccagtctcagttcggattggggtctgcaactcgaccccatgaagtcggagtcgctagtaatcgcagatcagcaacgctg

SEQ ID NO: 8 - DNA, 1406 bp, Pseudomonas sp.
  misc_feature (1309)..(1309): n is a, c, g, or t
  ribosomal RNA nucleic acid sequence
ctaacacatgcaagtcgagcggtagagaggtgcttgcacctcttgagagcggcggacgggtgagtaatgcctaggaatctgcctggtagtgggggataacgctcggaaacggacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcattaacctaatacgttagtgttttgacgttaccgacagaataagcaccggctaactctgtgccagcagccgcggtaatacagagggtgcaagcgttaatcggaattactgggcgtaaagcgcgcgtaggtggttcgttaagttggatgtgaaatccccgggctcaacctgggaactgcattcaaaactgtcgagctagagtatggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttgggagccttgagctcttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatccaatgaactttccagagatggattggtgccttcgggaacattgagacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatcccacaaaaccgatcgtagtccggatcgcagtctgcaactcgactgcgtgaagtcggaatcgctagtaatcgcgaatcanaatgtcgcggtgaatacgttcccgggccttgtacacaccgcccgtcacaccatgggagtgggttgcaccagaagtagctagtctaaccttcgggggg

SEQ ID NO: 9 - DNA, 1390 bp, Pseudomonas sp.
  misc_feature (1378)..(1378): n is a, c, g, or t
  ribosomal RNA nucleic acid sequence
cgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctttgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatccatctctggaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagctcgccagttttggatgcagttcccaggttgagcccggggatttcacatccaacttaacgaaccacctacgcgcgctttacgcccagtaattccgattaacgcttgcaccctctgtattaccgcggctgctggcacagagttagccggtgcttattctgtcggtaacgtcaaaacagcaaagtattaatttactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctgatagcgcaaggcccgaaggtcccctgctttctcccgtaggacgtatgcggtattagcgttcctttcgaaacgttgtcccccactaccaggcagattcctaggcattactcacccgtccgccgctgaattcaggagcaagctcccttcntccgctcgactg

SEQ ID NO: 10 - DNA, 1384 bp, Paenibacillus sp.
  misc_feature (201)..(201): n is a, c, g, or t
  misc_feature (1276)..(1276): n is a, c, g, or t
  ribosomal RNA nucleic acid sequence
ggctggctccttgcggttaccccaccgacttcgggtgttataaactctcgtggtgtgacgggcggtgtgtacaagacccgggaacgtattcaccgcggcatgctgatccgcgattactagcaattccgacttcatgcaggcgagttgcagcctgcaatccgaactgagaccggctttttaggattcgttccacctcgcggnttcactgcccgttgtaccggccattgtagtacgtgtgtagcccaggtcataaggggcatgatgatttgacgtcatccccaccttcctccggtttgtcaccggcagtcaccttagagtgcccacccgaagtgctggcaactaagatcaagggttgcgctcgttgcgggacttaacccaacatctcacgacacgagctgacgacaaccatgcaccacctgtctcaactttccccgaagggcacctaacgtatctctacctcgttagttggatgtcaagacctggtaaggttcttcgcgttgcttcgaattaaaccacatactccactgcttgtgcgggtccccgtcaattcctttgagtttcagtcttgcgaccgtactccccaggcggaatgcttaatgtgttaacttcggcaccaagggtatcgaaacccctaacacctagcattcatcgtttacggcgtggactaccagggtatctaatcctgtttgctccccacgctttcgcgcctcagcgtcagttacagcccagagagtcgccttcgccactggtgttcctccacatctctacgcatttcaccgctacacgtggaattccactctcctcttctgcactcaagtcacgcagtttccagtgcgatccggggttgagccccgggattaaacaccagacttacatgaccgcctgcgcgcgctttacgcccaataattccggacaacgcttgccccctacgtattaccgcggctgctggcacgtagttagccggggctttcttctcaggtaccgtcaccttgggagcagttactctcccaagcgttcttccctggcaacagagctttacgatccgaaaaccttcatcactcacgcggcattgctccgtcaggctttcgcccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcaggcccatcctcaagtgacagattgctccgtctttccagttcccntcaggcgaagaaaacaagtattcggtattagctaccgtttccggtagttgtcccaagcttgagggcaggttgcctacgtgttactcacccgtccgccgctaagtatca

SEQ ID NO: 11 - DNA, 1350 bp, Rahnella sp.
  misc_feature (159)..(159): n is a, c, g, or t
  misc_feature (1200)..(1200): n is a, c, g, or t
  misc_feature (1252)..(1252): n is a, c, g, or t
  misc_feature (1287)..(1287): n is a, c, g, or t
  misc_feature (1302)..(1302): n is a, c, g, or t
  ribosomal RNA nucleic acid sequence
caaggggggataacaactggaaacggtagctaatccgcatgacctcgaaagagcaaagtggggggatcttcggacctcacgccatcggatgtgcccagatgggataagctagtgggtgaggtaatggctcacctaggcgacgatccctagctggtctgngaggatgaccagccacactggaactgagacacggtccagactcctacgggaggcagcagtggggaatattgcccaatgggcgcaagcctgatgcacccatgccgcgtgtgtgaagaaggccttagggttgtaaagcactttcagcgaggaggaaggcatcacacttaatacgtgtggtgattgacgttcctcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacagaattcgccagagatggcttagtgccttcgggaactctgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtcccggattggagtctgcaactcgactccatgaagtcnggaatcgctagtaatcgtagatcagaatgctacggtgaatacgttcccgggncttgtacacaccgcccgttcacaccatgggagtgngttgcaaagaagtantagcttaaccttcgggagggcgcttaccactttgtgattcatgactgg

SEQ ID NO: 12 - DNA, 1454 bp, Pseudomonas sp.
  misc_feature (423)..(423): n is a, c, g, or t
  misc_feature (1322)..(1322): n is a, c, g, or t
  misc_feature (1417)..(1417): n is a, c, g, or t
  misc_feature (1425)..(1425): n is a, c, g, or t
  ribosomal RNA nucleic acid sequence
gctggcggcaggcctaacacatgcaagtcgagcggatgacgggagcttgctccttgattcagcggcggacgggtgagtaatgcctaggaatctgcctggtagtgggggacaacgtttcgaaaggaacgctaataccgcatacgtcctacgggagaaagcaggggaccttcgggccttgcgctatcagatgagcctaggtcggattagctagttggtggggtaatggctcaccaaggcgacgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagntaataccttgctgttttgacgttaccgacagaataagcaccggctaactctgtgccagcagccgcggtaatacagagggtgcaagcgttaatcggaattactgggcgtaaagcgcgcgtaggtggtttgttaagttggatgtgaaagccccgggctcaacctgggaactgcatccaaaactggcaagctagagtacggtagagggtggtggaatttcctgtgtagcggtgaaatgcgtagatataggaaggaacaccagtggcgaaggcgaccacctggactgatactgacactgaggtgcgaaagcgtggggagcaaacaggattagataccctggtagtccacgccgtaaacgatgtcaactagccgttggaatccttgagattttagtggcgcagctaacgcattaagttgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggaactctgacacaggtgctgcatggctgtcgtcagctcgtgtcgtgagatgttgggttaagtcccgtaacgagcgcaacccttgtccttagttaccagcacgttatggtgggcactctaaggagactgccggtgacaaaccggaggaaggtggggatgacgtcaagtcatcatggcccttacggcctgggctacacacgtgctacaatggtcggtacagagggttgccaagccgcgaggtggagctaatctcacaaaaccgatcgtagtccggatcgcagtctgcaactcgactgcgtgaagtcggaatcgctagtaatcgcgaatcanaatgtcgcggtgaatacgttcccgggccttgtacacaccgcccgtcacaccatgggagtgggttgcaccagaagtagctagtctaaccttcgggnaggacggntaccacggtgtgattcatgactggggtga

SEQ ID NO: 13 - DNA, 1456 bp, Pseudoxanthomonas sp.
  ribosomal RNA nucleic acid sequence
aggctaaccacatgcaagtcgaacggcagcacaggagagcttgctctctgggtggcgagtggcggacgggtgaggaatacatcggaatctaccttttcgtgggggataacgtagggaaacttacgctaataccgcatacgacctacgggtgaaagtgggggaccgcaaggcctcacgcgattagatgagccgatgtccgattagctagttggcggggtaatggcccaccaaggcgacgatcggtagctggtctgagaggatgatcagccacactggaactgagacacggtccagactcctacgggaggcagcagtggggaatattggacaatgggcgcaagcctgatccagccataccgcgtgggtgaagaaggccttcgggttgtaaagcccttttgttgggaaagaaatcctatcggttaataaccggtggggatgacggtacccaaagaataagcaccggctaacttcgtgccagcagccgcggtaatacgaagggtgcaagcgttactcggaattactgggcgtaaagcgtgcgtaggtggtggtttaagtctgctgtgaaagccctgggctcaacctgggaattgcagtggatactggatcactagagtgtggtagagggatgcggaatttctggtgtagcagtgaaatgcgtagagatcagaaggaacatccgtggcgaaggcggcatcctgggccaacactgacactgaggcacgaaagcgtggggagcaaacaggattagataccctggtagtccacgccctaaacgatgcgaactggatgttgggtgcaacttggcacccagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatccacggaactttccagagatggattggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggtagggacagagggctgcaaacccgcgagggtgagccaatcccagaaaccctatctcagtccggattggagtctgcaactcgactccatgaagtcggaatcgctagtaatcgcagatcagcattgctgcggtgaatacgttcccgggccttgtacacaccgcccgtcacaccatgggagtttgcttgcaccagaagcaggtagcttaaccttcgggagggcgcttgccacggtgtggccgatgactggggtgaatc

SEQ ID NO: 14 - DNA, 1463 bp, Erwinia sp.
  ribosomal RNA nucleic acid sequence
gtggtaagcgccctcccgaaggttaagctacctacttcttttgcaacccactcccatggtgtgacgggcggtgtgtacaaggcccgggaacgtattcaccgtagcattctgatctacgattactagcgattccgacttcacggagtcgagttgcagactccgatccggactacgacgcactttatgaggtccgcttgctctcgcgaggtcgcttctctttgtatgcgccattgtagcacgtgtgtagccctggccgtaagggccatgatgacttgacgtcatccccaccttcctccggtttatcaccggcagtctcctttgagttcccgacatcactcgctggcaacaaaggataagggttgcgctcgttgcgggacttaacccaacatttcacaacacgagctgacgacagccatgcagcacctgtctcacggttcccgaaggcactaaggcatctctgccgaattccgtggatgtcaaggccaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacggcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactcaagcttgccagtttcaaatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgggtaacgtcaatgacgaaggttattaaccctcatcccttcctccccgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccgttaccccacctactagctaatcccatctgggttcatccgatggtgtgaggcccgaaggtcccccactttggtcttgcgacgttatgcggtattagctaccgtttccagtagttatccccctccatcgggcagatccccagacattactcacccgtccgccactcgtcacccggagagcaagctcccctgtgctaccgttcgacttgcatgtgttaggcctgccgccagcgttcaatctgagcctgaatcaaactct

SEQ ID NO: 15 - DNA, 1460 bp, Pseudomonas sp.
  ribosomal RNA nucleic acid sequence
ccgtgggtaaccgtcctcccgaaggttagactagctacttctggtgcaacccactcccatggtgtgacgggcggtgtgtacaaggcccgggaacgtattcaccgcaacattctgatttgcgattactagcgattccgacttcacgcagtcgagttgcagactgcgatccggactacgatcggttttgtgagattagctccacctcgcggcttggcaaccctctgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtgtcagagttcccgaaggcaccaatccatctctggaaagttctctgcatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaaaatctcaaggattccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagctcgccagttttggatgcagttcccaggttgagcccggggctttcacatccaacttaacgaaccacctacgcgcgctttacgcccagtaattccgattaacgcttgcaccctctgtattaccgcggctgctggcacagagttagccggtgcttattctgtcggtaacgtcaaaattgcacggtattagcgtacaacccttcctcccaacttaaagtgctttacaatccgaagaccttcttcacacacgcggcatggctggatcaggctttcgcccattgtccaatattccccactgctgcctcccgtaggaatctggaccgtgtctcagttccagtgtgactgatcatcctctcagaccagttacggatcgtcgcctaggtgagccattacctcacctactagctaatccgacctaggctcatctgatagcgcaaggcccgaaggtcccctgctttctcccgtaggacgtatgcggtattagcgttcctttcgaaacgttgtcccccactaccaggcagattcctaggcattactcacccgtccgccgctgaatcaaggagcaagctcccgtcatccgctcgacttgcatgtgttaggcctgccgccagcgttcaatctgagccatgatccaaactc

SEQ ID NO: 16 - DNA, 1507 bp, Pseudomonas sp.
  ribosomal RNA nucleic acid sequence
ttacgggttacccctgtacgacttcaccccagtcatgaatcacaccgtggtaaccgtcctcccgaaggttagactagctacttctggtgcaacccactcccatggtgtgacgggcggtgtgtacaaggcccgggaacgtattcaccgcgacattctgattcgcgattactagcgattccgacttcacgcagtcgagttgcagactgcgatccggactacgatcggttttatgggattagctccacctcgcggcttggcaaccctttgtaccgaccattgtagcacgtgtgtagcccaggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtctcaatgttcccgaaggcaccaatctatctctagaaagttcattggatgtcaaggcctggtaaggttcttcgcgttgcttcgaattaaaccacatgctccaccgcttgtgcgggcccccgtcaattcatttgagttttaaccttgcggccgtactccccaggcggtcaacttaatgcgttagctgcgccactaagagctcaaggctcccaacggctagttgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccatactctagcttgccagttttggatgcagttcccaggttgagcccggggctttcacatccaacttaacaaaccacctacgcgcgctttacgcccagtaattccgattaacgcttgcaccctctgtattaccgcggctgctggcacagagttagccggtgcttattctgtcggtaacgtcaaaacaatcacgtattaggtaactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagccattacctcaccaactagctaatccgacctaggctcatctaatagcgtgaggtccgaagatcccccactttctcccgtaggacgtatgcggtattagcgcccgtttccggacgttatcccccactactaggcagattcctaggcattactcacccgtccgccgctcgccaccaggtacaagtacccgtgctgccgctcgacttgcatgtgttaggcctgccgccagcgttcaatctgagccatgaatcaaactcta

SEQ ID NO: 17 - DNA, 1449 bp, Ensifer sp.
  ribosomal RNA nucleic acid sequence
gagtttgatcatggctcagaacgaacgctggcggcaggcttaacacatgcaagtcgagcgccccgcaaggggagcggcagacgggtgagtaacgcgtgggaatctacccttttctacggaataacgcagggaaacttgtgctaataccgtatacgcccttcgggggaaagatttatcgggaaaggatgagcccgcgttggattagctagttggtggggtaaaggcctaccaaggcgacgatccatagctggtctgagaggatgatcagccacattgggactgagacacggcccaaactcctacgggaggcagcagtggggaatattggacaatgggcgcaagcctgatccagccatgccgcgtgagtgatgaaggccctagggttgtaaagctctttcaccggtgaagataatgacggtaaccggagaagaagccccggctaacttcgtgccagcagccgcggtaatacgaagggggctagcgttgttcggaattactgggcgtaaagcgcacgtaggcggacatttaagtcaggggtgaaatcccggggctcaaccccggaactgcctttgatactgggtgtctagagtatggaagaggtgagtggaattccgagtgtagaggtgaaattcgtagatattcggaggaacaccagtggcgaaggcggctcactggtccattactgacgctgaggtgcgaaagcgtggggagcaaacaggattagataccctggtagtccacgccgtaaacgatgaatgttagccgtcgggcagtttactgttcggtggcgcagctaacgcattaaacattccgcctggggagtacggtcgcaagattaaaactcaaaggaattgacgggggcccgcacaagcggtggagcatgtggtttaattcgaagcaacgcgcagaaccttaccagcccttgacatcccgatcgcggattacggagacgttttccttcagttcggctggatcggagacaggtgctgcatggctgtcgtcagctcgtgtcgtgagatgttgggttaagtcccgcaacgagcgcaaccctcgcccttagttgccagcatttagttgggcactctaaggggactgccggtgataagccaaaaggaaggtggggatgacgtcaagtcctcaagggccccttacgggctgggctacacacctgctacaatggtggtgacaatgggcaccgaaaaccgcgaggtcgacctaatctccaaaaaccatctcagttcggaatgcactctgcaactcgagtgcatgaagttggaatcgctagtaatcgcagatcagcatgctgcggtgaatacgttcccgggccttgtacacaccgcccgtcacaccatgggagttggttctacccgaaggtagtgcgctaaccgcaaggaggcagctaaccacggtagggtcagcgactggggtgaagtcgtaacagggtaaccg

SEQ ID NO: 18 - DNA, 1457 bp, Pantoea sp.
  ribosomal RNA nucleic acid sequence
gcagtcgacgcgcagcacagaagagcttgctctttgggtggcgagtggcggacgggtgagtaatgtctgggaaactgcccgatggagggggataactactggaaacggtagctaataccgcataacgtcgcgagaccaaagtgggggaccttcgggcctcacaccatcggatgtgccccgatgggattagctagtaggtggggtaatggctcacctaggcgacgatccctagcttgtctgagaggatgaccagccacactggaactgagacacgttaaaaaaatcatgcgggaggcagcagtggggaatattgcacaatgggcgcaagcctgatgcagccatgccgcgtgaatgaagaaggccttcgggttgtaaagtactttcagcggggaggaaggcggtgaggttaataacctcgccgattgacgttacccgcagaagaagcaccggctaactccgtgccagcagccgcggtaatacggagggtgcaagcgttaatcggaattactgggcgtaaagcgcacgcaggcggtctgttaagtcagatgtgaaatccccgggcttaacctgggaactgcatttgaaactggcaggcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccagagaacttagcagagatgctttggtgccttcgggaactctgagacaggtgctgcatggctgtcgtcagctcgtgttgtgaaatgttgggttaagtcccgcaacgagcgcaacccttatcctttgttgccagcgactcggccgggaactcaaaggagactgccggtgataaaccggaggaaggtggggatgacgtcaagtcatcatggcccttacggccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtacagggggaaacccg

SEQ ID NO: 19 - DNA, 1504 bp, Acinetobacter sp.
  ribosomal RNA nucleic acid sequence
ttacggttacccctgtacgacttcaccccagtcatcggccacaccgtggtaagcgtcctccttgcggttagactacctacttctggtgcaacaaactcccatggggtgacgggcggtgtgtacaaggcccgggaacgtattcaccgcggcattctgatccgcgattacaagcgattccgacttcatggagtcgagttgcagactccaatccggactacgatcggctttttgagattagcatcacatcgctgtgtagcaaccctttgtaccgaccattgtagcacgtgtgcagccctggccgtaagggccatgatgacttgacgtcgtacccgcatacctaaaatttgtcactggcagtatccttaaagttcccatccgaaatgctggcaagtaaggaaaagggttgcgctcgttgcgggacttaacccaacatctcacgacacgagctgacgacagccatgcagcacctgtatctagattcccgaaggcaccaatccatctctggaaagtttctagtatgtcaaggccaggtaaggttcttcgcgttgcatcaaattaaaccacatgctccaccgcttgtgcgggcccccgtcaattcatttgagttttagtcttgcgaccgtactccccaggcggtctacttatcgcgttagctgcgccactaaagcctcaaaggccccaacggctagtagacatcgtttacggcatggactaccagggtatctaatcctgtttgctccccatgctttcgtacctcagcgtcagtattaggccagatggctgccttcgccatcggtattcctccagatctctacgcatttcaccgctacacctggaattctaccatcctctcccatactctagcttcccagtatcgaatgcaattcccaagttaagctcggggatttcacatccgacttaaaaagccgcctacgcacgctttacgcccagtaaatccgattaacgctcgcaccctctgtattaccgcggctgctggcacagagttagccggtgcttattctgcgagtaacgtccactatccaagagtattagtctcagtagcctcctcctcgcttaaagtgctttacaaccataaggccttcttcacacacgcggcatggctggatcagggttccccccattgtccaatattccccactgctgcctcccgtaggagtctgggccgtgtctcagtcccagtgtggcggatcatcctctcagacccgctacagatcgtcgccttggtaggcctttaccccaccaactagctaatccgacttaggctcatctattagcgcaaggtccgaagatcccctgctttctcccgtaggacgtatgcggtattagcattcctttcggaatgttgtcccccactaataggcagattcctaagcattactcacccgtccgccgctaagataaggtgcaagcacctcatctccgctcgacttgcatgtgttaagcctgccgccagcgttcaatctgagccatgattcaaactcta

SEQ ID NO: 20 - DNA, 1463 bp, Enterobacter sp.
  ribosomal RNA nucleic acid sequence
aagtggtaagcgccctcccgaaggttaagctacctacttcttttgcaacccactcccatggtgtgacgggcggtgtgtacaaggcccgggaacgtattcaccgtagcattctgatctacgattactagcgattccgacttcatggagtcgagttgcagactccaatccggactacgacgcactttatgaggtccgcttactctcgcaagttcgcttctctttgtatgcgccattgtagcacgtgtgtagccctactcgtaagggccatgatgacttgacgtcatccccaccttcctccagtttatcactggcagtctcctttgagttcccggccgaaccgctggcaacaaaggataggggttgcgctcgttgcgggacttaacccaacatttcacaacacgagctgacgacagccatgcagcacctgtctcagagttcccgaaggcaccaatccatctctggaaagttctctggatgtcaagagtaggtaaggttcttcgcgttgcatcgaattaaaccacatgctccaccgcttgtgcgggcccccgtcaattcatttgagttttaaccttgcggccgtactccccaggcggtcgacttaacgcgttagctccggaagccacgcctcaagggcacaacctccaagtcgacatcgtttacagcgtggactaccagggtatctaatcctgtttgctccccacgctttcgcacctgagcgtcagtcttcgtccagggggccgccttcgccaccggtattcctccagatctctacgcatttcaccgctacacctggaattctacccccctctacgagactctagcttgccagtttcgaatgcagttcccaggttaagcccggggatttcacatccgacttgacaaaccgcctgcgtgcgctttacgcccagtaattccgattaacgcttgcaccctccgtattaccgcggctgctggcacggagttagccggtgcttcttctgcgagtaacgtcaatcggcaaggttattaaccttaccgccttcctcctcgctgaaagtactttacaacccgaaggccttcttcatacacgcggcatggctgcatcaggcttgcgcccattgtgcaatattccccactgctgcctcccgtaggagtctggaccgtgtctcagttccagtgtggctggtcatcctctcagaccagctagggatcgtcgcctaggtgagccattaccccacctactagctaatcccatctgggcacatccgatggtgtgaggcccgaaggtcccccactttggtccgaagacgttatgcggtattagctaccgtttccagtagttatccccctccatcgggcagtttcccagacattactcacccgtccgccactcgtcacccaagagcaagctctctgtgctaccgttcgacttgcatgtgttaggcctgccgccagcgttcaatctgagccatgaatcaaactct

SEQ ID NO: 21 - DNA, 1457 bp, Pseudomonas sp.
  ribosomal RNA nucleic acid sequence
tccgtggtaaccgtccccttgcggttagactagctacttctggagcacccactcccatggtgtgacgggcggtgtgtacaaggcccgggaacgtattcaccgtgacattctgattcacgattactagcgattccgacttcacgcagtcgagttgcagactgcgatccggactacgatcggttttatgggattagctccacctcgcggtttggcaaccctttgtaccgaccattgtagcacgtgtgtagccctggccgtaagggccatgatgacttgacgtcatccccaccttcctccggtttgtcaccggcagtctccttagagtgcccaccataacgtgctggtaactaaggacaagggttgcgctcgttacgggacttaacccaacatctcacgacacgagctgacgacagccatgcagcacctgtgtctgagttcccgaaggcaccaatccatctctggaaagttctcagcatgtcaaggccaggtaaggttcttcgcgttgcttcgaattaaaccacatgctccaccgcttgtgcgggcccccgtcaattcatttgagttttaaccttgcggccgtactccccaggcggtcgacttatcgcgttagcttcgccactaagatctcaaggatcccaacggctagtcgacatcgtttacggcgtggactaccagggtatctaatcctgtttgctccccacgctttcgcacctcagtgtcagtatcagtccaggtggtcgccttcgccactggtgttccttcctatatctacgcatttcaccgctacacaggaaattccaccaccctctaccgtactctagctcagtagttttggatgcagttcccaggttgagcccggggctttcacatccaacttgctgaaccacctacgcgcgctttacgcccagtaattccgattaacgcttgcacccttcgtattaccgcggctgctggcacgaagttagccggtgcttattctgtcggtaacgtcaaaacagcaaggtattaacttactgcccttcctcccaacttaaagtgctttacaatccgaagaccttcttcacacacgcggcatggctggatcaggctttcgcccattgtccaatattccccactgctgcctcccgtaggagtctggaccgtgtctcagttccagtgtgactgatcatcctctcagaccagttacggatcgtcgccttggtgagcctttacctcaccaactagctaatccgacctaggctcatctgatagcgcaaggcccgaaggtcccctgctttctcccgtaggacgtatgcggtattagcgttcctttcgaaacgttgtcccccaccaccaggcagactcctaggcattactcacccgtccgccgctgaatcatggagcaagctccactcatccgctcgacttgcatgtgttaggcctgccgccagcgttcaatctgagccatgattcaaact

SEQ ID NO: 22 - DNA, 1507 bp, Acinetobacter sp.
  ribosomal RNA nucleic acid sequence
tttagagtttgaatcatggctcagattgaacgctggcggcaggcttaacacatgcaagtcgagcggagatgaggtgcttgcaccttacctcagcggcggacgggtgagtaatgcttaggaatctgcctattagtgggggacaacattccgaaaggaatgctaataccgcatacgtcctacgggagaaagcaggggatcttcggaccttgcgctaatagatgagcctaagtcgcattagctagttggtggggtaaaggcctaccaaggcgacgatctgtaccgggtctgagaggatgatccgccaaactgggactgacacacggcccacactcatacgggaggcagcagtggggaatattggacaatggggggaaccctgatccagccatgccgcgtgtgtgaagaaggccttatggttgtaaagcactttaagcgaggaggaggctactgagactaatactcttggatagtggacgttactcgcagaataagcaccggctaactctgtgccagcagccgcggtaatacagagggtgcgagcgttaatcggatttactgggcgtaaagcgtgcgtaggcggctttttaagtcggatgtgaaatccccgagcttaacttgggaattgcattcgatactgggaagctagagtatgggagaggatggtagaattccaggtgtagcggtgaaatgcgtagagatctggaggaataccgatggcgaaggcagccatctggcctaatactgacgctgaggtacgaaagcatggggagcaaacaggattagataccctggtagtccatgccgtaaacgatgtctactagccgttggggcctttgaggctttagtggcgcagctaacgcgataagtagaccgcctggggagtacggtcgcaagactaaaactcaaatgaattgacgggggcccgcacaagcggtggagcatgtggtttaattcgatgcaacgcgaagaaccttacctggccttgacatactagaaactttccagagatggattggtgccttcgggaatctagatacaggtgctgcatggctgtcgtcagctcgtgtcgtgagatgttgggttaagtcccgcaacgagcgcaacccttttccttacttgccagcatttcggatgggaactttaaggatactgccagtgacaaactggaggaaggcggggacgacgtcaagtcatcatggcccttacggccagggctacacacgtgctacaatggtcggtacaaagggttgctacacagcgatgtgatgctaatctcaaaaagccgatcgtagtccggattggagtctgcaactcgactccatgaagtcggaatcgctagtaatcgcggatcagaatgccgcggtgaatacgttcccgggccttgtacacaccgcccgtcacaccatgggagtttgttgcaccagaagtaggtagtctaaccgcaaggaggacgcttaccacggtgtggccgatgactggggtgaagtcgaacaggggtaaccgtaaa

SEQ ID NO: 23 - DNA, 1504 bp, Bacillus sp.
  misc_feature (579)..(579): n is a, c, g, or t
  ribosomal RNA nucleic acid sequence
ggctcaggatgaacgctggcggcgtgcctaatacatgcaagtcgagcgaactgattagaagcttgcttctatgacgttagcggcggacgggtgagtaacacgtgggcaacctgcctgtaagactgggataacttcgggaaaccgaagctaataccggataggatcttctccttcatgggagatgattgaaagatggtttcggctatcacttacagatgggcccgcggtgcattagctagttggtgaggtaacggctcaccaaggcaacgatgcatagccgacctgagagggtgatcggccacactgggactgagacacggcccagactcctacgggaggcagcagtagggaatcttccgcaatggacgaaagtctgacggagcaacgccgcgtgagtgatgaaggctttcgggtcgtaaaactctgttgttagggaagaacaagtacgagagtaactgctcgtaccttgacggtacctaaccagaaagccacggctaactacgtgccagcagccgcggtaatacgtaggtggcaagcgttatccggaattattgggcgtaaagcgcgcgcaggcggttncttaagtctgatgtgaaagcccacggctcaaccgtggagggtcattggaaactggggaacttgagtgcagaagagaaaagcggaattccacgtgtagcggtgaaatgcgtagagatgtggaggaacaccagtggcgaaggcggctttttggtctgtaactgacgctgaggcgcgaaagcgtggggagcaaacaggattagataccctggtagtccacgccgtaaacgatgagtgctaagtgttagagggtttccgccctttagtgctgcagctaacgcattaagcactccgcctggggagtacggtcgcaagactgaaactcaaaggaattgacgggggcccgcacaagcggtggagcatgtggtttaattcgaagcaacgcgaagaaccttaccaggtcttgacatcctctgacaactctagagatagagcgttccccttcgggggacagagtgacaggtggtgcatggttgtcgtcagctcgtgtcgtgagatgttgggttaagtcccgcaacgagcgcaacccttgatcttagttgccagcatttagttgggcactctaaggtgactgccggtgacaaaccggaggaaggtggggatgacgtcaaatcatcatgccccttatgacctgggctacacacgtgctacaatggatggtacaaagggctgcaagaccgcgaggtcaagccaatcccataaaaccattctcagttcggattgtaggctgcaactcgcctacatgaagctggaatcgctagtaatcgcggatcagcatgccgcggtgaatacgttcccgggccttgtacacaccgcccgtcacaccacgagagtttgtaacacccgaagtcggtggagtaaccgtaaggagctagccgcctaaggtgggacagatgattggggtgaagtcgtaacaggggtaaccgta

SEQ ID NO: 24 - DNA, 1483 bp, Streptomyces sp.
  ribosomal RNA nucleic acid sequence
taccttgttacgacttcgtcccaatcgccagtcccaccttcgacagctccctccacaaggggttgggccaccggcttcgggtgttaccgactttcgtgacgtgacgggcggtgtgtacaaggcccgggaacgtattcaccgcagcaatgctgatctgcgattactagcaactccgacttcatggggtcgagttgcagaccccaatccgaactgagaccggctttttgagattcgctccgcctcgcggcatcgcagctcattgtaccggccattgtagcacgtgtgcagcccaagacataaggggcatgatgacttgacgtcgtccccaccttcctccgagttgaccccggcagtctcctgtgagtccccatcaccccgaagggcatgctggcaacacagaacaagggttgcgctcgttgcgggacttaacccaacatctcacgacacgagctgacgacagccatgcaccacctgtataccgaccacaaggggggcaccatctctgatgctttccggtatatgtcaagccttggtaaggttcttcgcgttgcgtcgaattaagccacatgctccgctgcttgtgcgggcccccgtcaattcctttgagttttagccttgcggccgtactccccaggcggggaacttaatgcgttagctgcggcaccgacgacgtggaatgtcgccaacacctagttcccaacgtttacggcgtggactaccagggtatctaatcctgttcgctccccacgctttcgctcctcagcgtcagtaatggcccagagatccgccttcgccaccggtgttcctcctgatatctgcgcatttcaccgctacaccaggaattccgatctcccctaccacactctagctagcccgtatcgaatgcagacccggggttaagccccgggctttcacatccgacgtgacaagccgcctacgagctctttacgcccaataattccggacaacgcttgcgccctacgtattaccgcggctgctggcacgtagttagccggcgcttcttctgcaggtaccgtcactttcgcttcttccctgctgaaagaggtttacaacccgaaggccgtcatccctcacgcggcgtcgctgcatcaggctttcgcccattgtgcaatattccccactgctgcctcccgtaggagtctgggccgtgtctcagtcccagtgtggccggtcgccctctcaggccggctacccgtcgtcgccttggtaggccattaccccaccaacaagctgataggccgcgggctcatccttcaccgccggagcttttaaccccgtcccatgcgggacagagtgttatccggtattagaccccgtttccagggcttgtcccagagtgaagggcagattgcccacgtgttactcacccgttcgccactaatccaccccgaaaggcttcatcgttcgacttgcatgtgttaagcacgccgccagcgttcgacctgagccatgatcaaactct

SEQ ID NO: 25 - DNA, 1511 bp, Streptomyces sp.
  ribosomal RNA nucleic acid sequence
ggagagtttgatcctggctcaggacgaacgctggcggcgtgcttaacacatgcaagtcgaacgatgaaccacttcggtggggattagtggcgaacgggtgagtaacacgtgggcaatctgccctgcactctgggacaagccctggaaacggggtctaataccggatactgatcctcgcaggcatctgcgaggttcgaaagctccggcggtgcaggatgagcccgcggcctatcagctagttggtgaggtaacggctcaccaaggcgacgacgggtagccggcctgagagggcgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggaagaagcgaaagtgacggtacctgcagaagaagcgccggctaactacgtgccagcagccgcggtaatacgtagggcgcaagcgttgtccggaattattgggcgtaaagagctcgtaggcggcttgtcacgtcggttgtgaaagcccggggcttaaccccgggtctgcagtcgatacgggcaggctagagttcggtaggggagatcggaattcctggtgtagcggtgaaatgcgcagatatcaggaggaacaccggtggcgaaggcggatctctgggccgatactgacgctgaggagcgaaagcgtggggagcgaacaggattagataccctggtagtccacgccgtaaacggtgggcactaggtgtgggcaacattccacgttgtccgtgccgcagctaacgcattaagtgccccgcctggggagtacggccgcaaggctaaaactcaaaggaattgacgggggcccgcacaagcggcggagcatgtggcttaattcgacgcaacgcgaagaaccttaccaaggcttgacatacaccggaaaaccctggagacagggtcccccttgtggtcggtgtacaggtggtgcatggctgtcgtcagctcgtgtcgtgagatgttgggttaagtcccgcaacgagcgcaacccttgtcccgtgttgccagcaggcccttgtggtgctggggactcacgggagaccgccggggtcaactcggaggaaggtggggacgacgtcaagtcatcatgccccttatgtcttgggctgcacacgtgctacaatggccggtacaatgagctgcgataccgcgaggtggagcgaatctcaaaaagccggtctcagttcggattggggtctgcaactcgaccccatgaagtcggagtcgctagtaatcgcagatcagcattgctgcggtgaatacgttcccgggccttgtacacaccgcccgtcacgtcacgaaagtcggtaacacccgaagccggtggcccaaccccttgtgggagggagctgtcgaaggtgggactggcgattgggacgaagtcgtaacaaggtagccgtaccggaaggtgcggctggat

SEQ ID NO: 26 - DNA, 1481 bp, Actinokineospora sp.
  ribosomal RNA nucleic acid sequence
gagtttgatcctggctcaggacgaacgctggcggcgtgcttaacacatgcaagtcgagcggtatggcccttcggggtacacgagcggcgaacgggtgagtaacacgcgggtaatctgccccgtactctgggataagccttggaaacgaggtctaataccggatatgactccacatcgcatggtgtggggtggaaagttccggcggtacgggatgaacccgcggcctatcagcttgttggtggggtagtggcctaccaaggcgacgacgggtagccggcctgagagggtgaccggccacactgggactgagacacggcccagactcctacgggaggcagcagtggggaatattgcacaatgggcgaaagcctgatgcagcgacgccgcgtgagggatgacggccttcgggttgtaaacctctttcagcagggacgaagggcaactgacggtacctgcagaagaagcaccggctaactacgtgccagcagccgcggtaatacgtagggtgcgagcgttgtccggaattattgggcgtaaagagctcgtaggcggtttgtcgcgtcggccgtgaaaacctggggctcaaccctgggcctgcggtcgatacgggcagacttgagttcggtaggggagactggaattcctggtgtagcggtgaaatgcgcagatatcaggaggaacaccggtggcgaaggcgggtctctgggccgatactgacgctgaggagcgaaagcgtggggagcgaacaggattagataccctggtagtccacgccgtaaacggtgggtgctaggtgtggggaacattccacgttctccgtgccgtagccaacgcattaagcaccccgcctggggagtacggccgcaaggctaaaactcaaaggaattgacgggggcccgcacaagcggcggagcatgtggattaattcgatgcaacgcgaagaaccttacctgggcttgacatgcactggaaaccagtagagatgttggcccccttgtggccggtgtacaggtggtgcatggctgtcgtcagctcgtgtcgtgagatgttgggttgagtcccgcaacgagcgcaaccttcgttccatgttgccagcacgtaatggtggggactcatgggagactgccggggtcaactcggaggaaggtggggatgacgtcaagtcatcatgccccttatgtccagggcttcacacatgctacaatggtcggtacagagggctgcgagaccgtgaggtggagcgaatcccttaaagccggtctcagttcggatcggggtctgcaactcgaccccgtgaagtcggagtcgctagtaatcgcagatcagcaacgctgcggtgaatacgttcccgggccttgtacacaccgcccgtcacgtcatgaaagtcggtaacacccggagcccatggcccaacagttcactgagggagtggtcgaaggtgggactggcgattgggacgaagtcgtaacaaggtaaccgta

SEQ ID NO: 27 - DNA, 927 bp, Pantoea sp.
  Marker nucleic acid sequence
gggctgaggaatccccacaaaaaaagacaggcacagggttttatgatctacttattgcgccaacaaaaaacagatcgaaccctatgcctgcacgccaattatgccagaagtttttccgcaacgctttagcctccgttcactgctaccgccgcaacgccctgctggacgccgcggcagccctgtcaggcgacgccccgctcacgctgaccggcatcgggcgcagcctgcccggcagggcgcgcgtcaaggctaaaatcaagcgcgtcgaccgcctcctgggcaacgagcggctacaccgcgagctgcccctcatcgcaaaaaatattgccgccatgctgacggagcacctcagctggtgcgtgattgccgtggactggagcggctacgcctggtcggcgtttcacgtgctccgcgcaagcctggtcttcgacggccgggccatcccgttgatgagccgcgtcgtgccggaagagcagctgaacagcctgcaggttcaggctgacttcctggacgcgcttcacgccgccatcggccccggcaggcgggtcgtcgtcctgaccgacgcgggcttcaagagtgcctggttccggcacgtcagcgcgctgggctgggattttatcggccgggtacgcggcaacaaccggttcaggctggatggcgacggtcaggcctggcaggaggccggaagctgcgccggggtgaccggcaaaccggccgggctgggcagcggcgagctgttccggacgcggaccgcgcgctgcgcggggtacttttacggctataaaaagccgtccgcccggcgtaaggcccggcgcgcacggggccggccgcactacgcggcggcgaaccgcgagcacagcgcgtcggcgaaggagccgtgggtgctgttctgcagcacagacaggtacagcgcaaagacggtcatcc

SEQ ID NO: 28 - DNA, 328 bp, Pantoea sp.
  Marker nucleic acid sequence
tctgatggctcaaacacagcgacctcgggtgggaacacgtttttcaggtcctcgagcactgtcagccgggtgcctgggccaaaatactgcgtatctgtgctcacagcctggaggcccaggacaaaaaccggtgctgcccgctctgctgtcccagactcagctcgggtccttcccaccgctgagagccccatcgctctcccagcacccagaaccaggagtcctccgcccagacgcccagcagcgcctctcagcacagcccctctcagagtggggcagctgcttacctctccccatataagaaacggctgcgaaggttatgtgctctctg

SEQ ID NO: 29 - DNA, 233 bp, Bacillus sp.
  Marker nucleic acid sequence
gtacagacacaattggtacgtagaccatgattatattctcaggtattgtcgcagcttttgaaatgagaaatcccgacgtgtacatcagcatagaatcagagaataccgcaatggctcctaaaaataaaaccaaggcaaacaggttttgattctccttgatataaggtataaaccagcatttactgaccatgtgtgaccgcccccatttgctcgtgtagcatttgatagtacac

SEQ ID NO: 30 - DNA, 666 bp, Pseudomonas sp.
  Marker nucleic acid sequence
ggtgtcgatggcaccgctgtcgtttgccgagcctgtgttgccggctgcgtccgtgactgtggcatcaacggtgtaagggccctcagccaggccagccggtgcatcaaccgagtaagtgccgcctgctaccacgatcgcagttgtggtcagaactgtgctgccttgggtaaccgtcaacgtaacggtactgccaaccggcgcatcggtcgtgccggtgatgatgggcgtcgtgtcgttgctgattgcaggcgcatcgacggtgatcagcggcgcggtggtgtcgatggcacccgagtcgttggccgagccggtgttgccagctgcgtcggtgactgtggcatcaacagtgtaagagccttcagccagcccagccggtgcatcagccgagtaagtgccgcctgccaccacgattgcagtggtggtaagaacggtggtgccctgggtaaccgtaagcgtgaccgtgctgccagccggcgcatcagtggtaccggtaatggtcggcgtcgtgtcgttgctgattgcgggtgcatcgacggtgatcagtggggcggtggtgtcgatggcacccgaatcgttggccgagccggtattgccagcagcatcggtaacggtggcatcaatggagtacggcccctcagccaatggctgattcagctcgacgttgta

SEQ ID NO: 31 - DNA, 587 bp, Pseudomonas sp.
  Marker nucleic acid sequence
ggccagaaccggcgtggtcacggtaaagctggtattgccctgggtaaccgtcaacgtgaccgtgctgccaaccggcgcatcggtagtgccggtaatatttggagtcgtgtcgttgctgattgcgggtgcatcaacggtgatcagcggcgcggtggtatcgatggcaccgctgtcattagcggagcctgtgttgccggccgcgtcggtaactgtggcgtcgacggtgtaagagccttcagccaagccagccggtgtatcaaccgaataagtgccgcctgccaccacgatcgcagtggtggtcaggacggtagcgccttgggtgatggtcagcgtgactgtgctgccaaccggcgcgtcggtcgtgccggtgatggtcggcgtcgtgtcgttgctgatggcaggcgcatcgacagtgatcagcggggcggtggtgtcgatggcaccgctgtcgttagcggagccagtgttgccggcagcgtcggtgactgtggcatcaacggaatacgggctctcagccaatggctgattcagctcgacgttgtaggtgccgccggccagaaccggtgtagttaccgtaaaggtcgtgt

SEQ ID NO: 32 - DNA, 779 bp, Pseudomonas sp.
  Marker nucleic acid sequence
tcgagcagcttgcggaacatctcaacaccggtacaggtggtcttggtggtgtcacgcagaccaacgatctcgatttcttcctggatcttgacgataccgcgctctacacgaccggtcactacagtgccgcggccggagatcgagaatacgtcttcgatcggcatcaggaacggacggtcgattgcacgaaccggctccgggatgtagctgtccagcgtttcgacgagcttcttgacggcagtggtgcccatctcgttgtcgtcctggccgttcagagccatcagtgcggaaccgatgatgatcggcgtgtcgtcgcccgggaagtcgtaagtgctcagcagatcgcgcacttccatttccaccagctccagcagctcggcgtcatcaaccatgtcagccttgttcaggaacacgacgatgtacggtacgcctacctgacgcgacagcaggatgtgctcgcgagtctgaggcatcggaccatcggcagccgaacaaaccaggatagcgccgtccatctgggcagcaccggtgatcatgttcttgacgtagtcagcgtgacccgggcagtcaacgtgcgcgtagtgacgtacggcagagtcgtactcaacgtgcgcagtgttgatggtgataccacgagccttctcttccggcgcgctgtcgatcttgtcgaagtcgaccttggccgaaccgaatacttcggagcagacgcgggtcagagcagcggtcagagtggttttaccgtggtcaacgtgaccgatggtgccaacgttgacgtgcgg

SEQ ID NO: 33 - DNA, 992 bp, Sphingobium sp.
  Marker nucleic acid sequence
agctggtcgaccaatttgcgcgccgtcgcgccgatcgacgcgacctcggaaatccagtttcgcggcaccctgttccaggataaccgcaccctgcgctttgccggcgccgacagcatgagccagggccaggacgccagcatccgctatatcagccgcggccgctggcaggtcgatgcgctcgcctatctccaggcgcgcaacttctcgaacatcgtcatctcctccaccagcttccgcaagtcgctcgaccagcgcaacaccccctcgaccgggatcggcggcaagatcgagctgcgcccgccggtgggcgaggagcatgtgctgcgcatcggcgtggacacccgctttgccaccggcgacatgtatgaggatgcctataatgcgggccttgccaccaacccgctgacatcgcgccgccatgccggtggcgaccagatcacgtccggcatcttcgcagaggatgactggacgattggaaatctggtcctgaccggtggcatacgcgccgatcgctggtcgatccgcaacggcttctacaaggcggtcagtgcagccggcctggtgacgcaggacagcagctacgccaaccgatccgactgggaattttcaggccgggtaggcgcgctctatcatctgagcgatgcggtggcgctgcgcggcgcggcctatagcggcttccgcctgcccacgctcaacgagctgtatcgccccttcgtggtcttcccgatcaccacccaggccaatgcggcactaaagccggaaaagctgaaaggtgtggaggggggcatcgatctgaccccggcatcgggcgtgcaactctccgccacgctcttctacaaccggctcgacgatgcgatcgccaatgtcaccatcgccaccaacacccgccagcgccagaatgtgaacgcgatcgtggcgaagggcgtggaactgaccgccagcgcgcagctgcccgccaatttctcgctgctcgcttcctatgcctatagcca

SEQ ID NO: 34 - DNA, 932 bp, Sphingobium sp.
  Marker nucleic acid sequence
tgccctttccgagcgccctgcggcggctgcaatctcagtcgtgccgtggaccggggaggccggttcgatcaggatgacatggttggccgtctcccgaaaggtcaggccgctgcccttgagcagccgccgcagggcgctcggaaagggcatgacgcccgacagccggtgcgacgtggccgaactggccagcgcggatcggaaaacgatctgccgaccggcctgcagggccaaccgctggagcgcggtttcgatcttctggcggggtatgtcgaacgggatatcggcgctattccgggcagcggccgactggggaagaaggcaggctgcatatatcgttggtacgaccatggtcgccatccggttcagcgcgcggcgctttgggcgcgcgctgccggctcctgtccacctctgtaccactcgcaagccttattcatccgacccgatgttcagaacgagcgatcgcgcgctttccatcatgagctgcggccgttggctgaccgaaaaggtacaagatcatggatcgcccctcgatgggcacgtggccttgcgcgcccgcgccctgtggaaatggagtggagcgcatgacgtcgtcgtctttgcgaaggaaaatgcgacgatggccgcggggctaggctgtagcgcaacgggctgagccggtgaattaagttattctgttgcttctacaagtcggcgtgggggcagggaaggcatcccttgatccggggtaggaggcctatgtgatcgaaggggacagcgtcctgtgccgccaccgtcaaggccccatcctcggggggggcagatggtcgcaggaaatgagcgcgaatgacaattttcgatttttgtcagattttttcaaaaaagtgtttgaccgaatcctggggtcggcctagagggcttttcaccggacggggcgctgccaaacggaagcgctc

SEQ ID NO: 35 - DNA, 672 bp, Sphingobium sp.
  Marker nucleic acid sequence
aggggtagttggggactgtcggcatttgctcgcacaggggcgtttgctgccgttccgcttgcaatccattccacgccaagcctgttgaatgttctgcgagcacagacgagatatgagcatatgttgcacacagaaatcatttcagagttacttgatgaccattcgactctgctcgtccagacgggacggacgccgatcgacatgaaataccaacaaattcgtcagtggagcgccgatgccaatataccggaagttgagcttaccgagcggttagccctcgatgtagcagcaagatatgcgcagagagaaatcgattacgggccagcggactggattatcaacaatctgttcctcgcgatgaccgacgaccctttgagtaattggtcagatttattttttgacatctacaacgcttttgatagcggggaatataggcgggatggagacggtgacgttgaaccgagcgaaaaatacactcgcccagaaattgcacgaattctctcgatgaagcggtgatgctttggatgacggcaagaggctggctaagcgccaggctgttttccagtccttcataccccttgctccgcgttcttcgcggttccgcgtaattcattttttgcgcggagccgcgaagagcgcggagggcaaagaaggaagggcgactttgggtgg

SEQ ID NO: 36 - DNA, 894 bp, Lysobacter sp.
  Marker nucleic acid sequence
cgtcaattcgttgcgggcaaccttgatcctggtcgccgcatcgaccttgtcgttcggcaacgccaacatctacagccggtggcacaggagattcgtgatgaagttgatccttttttttgctcttgtctcggcttcgctgcctgccttagctcaatgtccagcaggaataccatctgctggtaacccggggtgcattccgccggaccaaactaattcgccctactatcaatcagggggctctagtcaaagtgctccaactgctcgttgggatgatcgttggggcgcaatcgccattgatgatcgagatggtggtgttggtctggggaccgctcagatgatgaccaccgaacgaaaggcaaaaaagacagcgttggatgaatgccgggcaaaaggtgggactagttgccgcgtgattttggcgtactacaaccaatgtggagttgtggttgctggtgatgctgggtggaattcgaccagtgcaggttctatcgatgaggctgcgaagcggagttttgaaaagtgtgaaaaaagcggcttgaagaactgcaaggtgtactactcgaattgcagtctcgctgaacgaattaggtaaatggtttttgttgcactgtttgtaaatgtagagcgctgcttgtcgatttctgaacttgccttgaggtaaggatgtacaggatgttgcttttgtcgtgatgcttcccccgatccgcggccatccggtgcaaggactgcatcgtgcatcctccgtcaccgtgggcgctcgccacgtgcgcgacccgatagcgataacgtcgtcggccggcgccagcggcaatgatcgtgtcgcctccccgtaaactcgctccaaccctaaccagaggtctccaggcaaactagccccaaggagacccagatgcg

SEQ ID NO: 37 - DNA, 784 bp, Lysobacter sp.
  Marker nucleic acid sequence
tgatcgagccggccgcccaggttttggccagctgcaaggtcgcagcccgggtgttgacgaaggtgcatgcgaactggctgttcggcaacaccgacgccgcgggaatggtgtaggtgcgcgttgcggcatcgaacgtgcccgatatcgcattgccattggctcggtccgtgcagttccagcccgccagcaccgtcgtcgtccagccgccgcctggatcggtctcggtaatcgtggtggcggtattcgccgccgtcagattcaccagcgccgattcgacttcggtacccgccgtcaccgtagtcacgctctgacccggatagccgttgctgccggtgaacgcgaacgtgccggtacctccgtcggaacgcttccagatccgcatctgcggcacccgggtgttgacgaaggtgcacgcgaactggctgttgggcagcaccgacgccgcgggaatcgtgtaggtgcgcgttgcggcatcgaacgtgcccgatatcgcattgccattggctcggtccgtgcagttccagcccgccagcgccgtcgtcgcccagccgccgcctggatcggtctcggtaatcgtggtggcggtattcgccgccgtcagattcaccagcgccgattcgacttcggtacccgccgtcaccgtggtcacgctctgacccggatagccgttgctgccggtgaacgcgaacgtgccggtacctccgtcggaacgcttccagatccgcatctgcgggcgacgcacagtattggtgtaggtacacaccgccgccgtgccgctggtgat

SEQ ID NO: 38 - DNA, 751 bp, Lysobacter sp.
  Marker nucleic acid sequence
gaacgcttccagatccgcatctgcgggcgacgcacagtattggtgtaggtacacaccgccgccgtgccgctggtgatggtcagcgtgttggtctgctggccattggtgcccgacagcgtgtgcccgccggtacaggtcacctcggtggtgtagttgcccacattggtgccgccctcagccggcagcgtgatcacgttgccgatcgtcaccgccaccgccgcgccactgttggccgcggtcggcgcagtcgcgttgaacgacaccgtgttgttggtgccgcccgtggtcgcgccgatcgtcacctggttgccggcgatcgagccggccgcccaagccttggccagttgcaacgtcgccgtgcgcggggtgttggtgtaggtacacaccgccgccgtgccgctggtgatggtcagcgtgttggtctgctgcccgttggtgcccgacaaggtgtgaccgccggtacacgccagcgtggtggtgtagttgcccgcgttggcgccgccctcggccggcagcgtgaccacgttgccgatcgtcaccgtcactgccgcgccgctgttggccgcggtcggtgcggtcgcgttgaacgacaccgtgttgttggtgccgcccgtggtcgcgccgatcgtcacctggttgccggcgatcgagccggccgcccaggccttggccagctgcaacgtcgccgtgcgcggggtgttggtgtaggtacacaccgccgccgtgccgctggtgatggtcagcgtgttgg

SEQ ID NO: 39 - DNA, 621 bp, Pseudomonas sp.
  Marker nucleic acid sequence
cggggtggggcgggggagggccgcgaggggggtgccccgggcgtggggggggcggcggcgcctcgtccagccgcggcgcgcgcccagccccgattcgcgccccagcccgaccgacccagcccttagagccaatccttatcccgaagttacggatccggcttgccgacttcccttacctacattgttccaacatgccagaggctgttcaccttggagacctgctgcggatatgggtacggcccggcgcgagatttacaccctctcccccggattttcaagggccagcgagagctcaccggacgccgccggaaccgcgacgctttccaaggcacgggcccctctctcggggcgaacccattccagggcgccctgcccttcacaaagaaaagagaactctccccggggctcccgccggcttctccgggatcggtcgcgttaccgcactggacgcctcgcggcgcccatctccgccactccggattcggggatctgaacccgactccctttcgatcggccgagggcaacggaggccatcgcccgtcccttcggaacggcactcgcccatctctcaggaccgactgacccatgttcaactgctgttcacagatcggaagagcacac

SEQ ID NO: 40 - DNA, 551 bp, Gordonia sp.
  Marker nucleic acid sequence
gcaccgacacgttgatgcccgcgtggcggtaccactcgtttttcaccaacaccaccgacgacaccgtcaccgccgacctcatccaccgccgccacgccatcatcgaaaccgtgttcgccgacctcatcgacggaccactggcgcatctgccctccggacggttcggcgccaacgccgcctggctggccctgaccgcgatcagccacaacctgatgcgcaccctcgccgcactcaccaacacccccacactgcgagccgcgcggggcgctacgctgcgccgcacactcatcgcgatacccgcccgactagcccgaccagcacgaacaccggtgctgcacctaccccgacactggcccacccaacacgccttcaccaccctgtgggccgcggccaacaccacctgacctgacgtcgccacccgcccacccacgaccaaggaaccccagtgaaaaggctgacacaggtcagcgaataccacacgcccacaacacaatcaggcgatcacgcgccccaaccccaccccacctgaactccttccacggatccaggct

SEQ ID NO: 41 - DNA, 538 bp, Gordonia sp.
  Marker nucleic acid sequence
cgaagagaccggaacgagccctttgcggtatctcttcacgggcggcgaggttctatccgatgttctagcagcggaagtgcaccaccaagaaccgaacttgcggctgctgaaccactacggtccgacggaaaccaccgttgacctcacttcagcgcagaatgagcctggtagatcggtaacaatcggtcgtccggcatggaacactgcgacctgggtcctggattcgcgtctgaatcgggtaccggtcggtgttccaggtgagctttacctaggtggtatccagctggcacgcgggtacgcctcgagatcggatctgactgcggcgcgtttcgttgccagtcctttcggcccccccgggtctcggctgtatcgcaccggcgaccttgtgcgatggaataccgaaggtgagatcgagtatctgggccgcaccgacttccaggtgaagttgcgtgggcagcgcattgagctcggtgagatcgaagcggtgatcgccactgcgcccggggtggtgcacaccgcggctacggtcatcgacgca

SEQ ID NO: 42 - DNA, 538 bp, Gordonia sp.
  Marker nucleic acid sequence
tgtggagaaggagattccgcgttctctggcctcgacgatcaccgcgtcggcgagcggatcaccctgcgtcagacccgcctcgaggcgctcgacgatggcgggatgccgctctcggacggcctcccaattgcgtgccccggtcaccgctgccgcgtccatgccccatgatcaccccggcgactgggccggtcaaggacggctgactcccagacgtcgactcgactgccagacctcgaccgaagtctggcggtcgagttgacgtctgtcggtggcagcgccggacgaatgcggacacgctgccgcggctgactatgctcaccgtggggaccgtcgtcgagcggacccttcgcccccatagcccaattggcagaggcagccgacttaaaaagtgcacagaaaattcgcgttttcgcactcatccggtgtttttaccggggtcgaggtcgaagttgacacctacagttgacacatgtcgggtgctaagtcggtgccccaggcgggactcgaacccgcaacacagacattttaagtgtcctgc

SEQ ID NO: 43 - DNA, 476 bp, Pseudomonas sp.
  Marker nucleic acid sequence
ggtcgccatcaccacgcccaaccatctgcacttcccgatggccatggccgcactggcggcgggcaaatcggtgtattgcgaaaagcccctggcggtgtccctggagcaggccgaacagatgcgccaggccgccaaggccgccggggtggtgacgcgggtcggctacaactaccagcacaacccgatcattcaatgggcgcgggagatgatccagcgcggcgagctggggcagatcatcagtttccagggcgaattcagcgaagacttcatggccgatccgacctcaccgtggtcatggcgctgcgaagcgagccatgccggtggcgcgctggcggacctgggcagccatttgctggccatggcccggcacttgttgggcgaggtcgaggccgtctgtgccgacagccagaccgtgcaccaccaacgcccggccagcgccggcaatgctgaacaacgggctatcgcggtggatgacc

SEQ ID NO: 44 - DNA, 466 bp, Pseudomonas sp.
  Marker nucleic acid sequence
ggcgctgctgagccaagatgagctactcggtcgtctgccagcgttgacggtgccggtggtgctgctcgaccgcgccgagcgcgtgcgttgcgggattgccggcggtcgtgacgacaatccgctcgttgccgccctgggtgcgcgccacctggcctacgtcatctacacctccggttccactggccatcccaaaggggtgatgatcgaacaccggggcctggtcaattattgcctcgatgcggtccgcttgttcggcttgacgccggcggacacggtgctgcaacagaacaccttgaacttcgacctgtcggtggaagaaatcttcccggcgttgttggccggcgccacgctggcgcccacccggcaactgtttggcagcgccgagttgcaacaaaatggcgatattcgcccgaccttcctgcacctgaccgcggcccattggcataccctcgtggccgagtggcac

SEQ ID NO: 45 - DNA, 461 bp, Pseudomonas sp.
  Marker nucleic acid sequence
tggccttcatcacttcggcgaaccctgaggcggcgatcatgatgaagccaatcatcgccatcatcttcatgccttcggtgaacaagtcgtccgtctcacgccagcgcaccacccccgacagcgagaaaatcaggaacccggccagggcgccgaggatcatcgagtccaccagcagttgcaccgcgaacgccgccgccaccgcaacgccggccacgatcaggctcagcgggttgtagtgcacggccacttgctcggccttggcgatcttcttcaggtcgtagacgcgtttcttgcggtacgtgataaacgacagcaccaggccaaacaccatgcccaacgccgggatacccatggcatgggtgatattgatgccactgatgtcgacgccgctgcgggcgacattggccagcaggatttcattgaggaagatattgccgaagcccaccggcaggaacatatag

SEQ ID NO: 46 - DNA, 439 bp, Pseudomonas sp.
  Marker nucleic acid sequence
ttgccgggcggtgctggcaacggtgtcgaccacccgttgataaccggcttgcatggcattgaatgccgccgccatctggcccacttcgtccttgcaggccaggggcacgcgggcggacaggtcgccggttttttccacgtgcagcatcacgtccttgagcgtgttgagctggctgagcaaaaagcggatcaacagttgcgacgcgcccaacatcgccagcatcaacacgaacaccgccacggcgtattggctgaagcgttcaccgaacacttggctcaggcttgcaccatgggccaatacggcgagttgctggccgtcgcctcgacggatcacttgggcgcccagcaaagggttgtcgccaaacagcagtccggggtcgagttctacccagccgttggcctgggtcagcgaaggcaatgattgttcattgataacggga

SEQ ID NO: 47 - DNA, 436 bp, Pseudomonas sp.
  Marker nucleic acid sequence
ccctgggggtgggcatcggcgattacgtggccttcgatcccctgccggaattcaccgaaagcggtcacatcagcgcccggcacctggacgacaaggccggggtcgcggccctgctggcggcgctcaaggcgattgtcgacagtggcgaggaactgctgatcgactgtcatccgctgttcactatcaccgaggaaaccggcagcggcgcagcggccgcactgccttgggacgtcagcgagtttgtcggcatcgacatcgcccccgtcgcacccggccagcactcgagcgaacattcggtcagcgtggcgatgcaggactccggcggcccctacgactatcacctgtcgcggcacctgctgaaactggccaaggaaaacgacatctcggcacgccgcgacctgttccgctactacttcagcgacgctcattcggccat

SEQ ID NO: 48 - DNA, 491 bp, Pseudomonas sp.
  Marker nucleic acid sequence
tcgaagttgccgccggatgcatccttgatcgtgacttcgactttgccagcgtctttgtagacgtcatctttcggcgcgtcgacggtcacggtgccggtggttttgccggcctcgatggtgatgacggagccgttgctcaacgtcacggtcaccggggtgccggccgggttggtcagggtggcggtgtaagtgatcgagccaccttcggccaccgagtcagtggcgctcagggtcagattagtggtatcgacggtgtcagtcacggtagtgcttaccggagtcttgtcggccaccagattttcgtagttaccgccgctcacaccggtgatcgcattggtcagcggagtgtgaccgttcaacgcgtcattcggcgcagtagtggtgatggtgccggtggttttgccgacttcgatagtgatcgactgaccattggccagggtcacggtcacaggcgaaccagtgacaggtgcgccaacagtggcggtgtacgt

SEQ ID NO: 49 - DNA, 319 bp, Pseudomonas sp.
  Marker nucleic acid sequence
ccgtcgttgccgaccagacggttgttgaggttgttgccggtggcattgatgtcgctgctgccggtcaacagcaggttctcgacgttggcggtgagggtgtagtcgatcgaagcgaatacacggtcgggagccgtgagcgaggcatcggtttcgatgaccaggtcaccgacgttgtccacgtaataggtgtcgataccggcgccaccttccatgcggtcggcaccaaggccaccatcgaggacgttgttgccgtcgttgccaaccagacggttgtcgagctcattgccggtggcattgatgttgccgttgccgatcagca

SEQ ID NO: 50 - DNA, 212 bp, Pseudomonas sp.
  Marker nucleic acid sequence
tcgataccggcgccaccttccatgcggtcggcaccaaggccaccatcgaggacgttgttgccgtcgttgccaaccagacggttgtcgagctcattgccggtggcattgatgttgccgttgccgatcagcaacaggttctcgacgttggcggtgagggtgtagtcgatcgaagcgaatacacggtcgggggccgtcagcgaggcatcggtttc

SEQ ID NO: 51 - DNA, 498 bp, Paenibacillus sp.
  Marker nucleic acid sequence
gggcacaagaaatcgacgaaagcccccaccggataacagctgcccagtgatgatgattgacggaataagagcaatcgcataagcagcaaaagccatggatagcatccctggtgtgagatgccactgttgcttgtatatcgcatagatgggtgaaacaagattaagacccatcactgcgataaacagaccatatgcggctgaccaataggagaaacgcttcgagcgtgtgcgcaccttgcccagacagacgacatccggtagctgctgtaaggaatacgagattctctccacaccttcatcatccaattcagcttgaatgcgtacgcgggacacatgccgatctcgcgatcttatcgtggcaatattgtcaatattgatccccagactggcaaacaaattggatatgcgagccagcactccggggtgattgttcaccgtcaccgacaagacacacgccctcatggctctctcctcctttagataccgtaacagaatatg

SEQ ID NO: 52 - DNA, 491 bp, Paenibacillus sp.
  Marker nucleic acid sequence
tacggcagatcggctggtattgttgttcccgtttccacacccccataccaaggatgtacatacgagtacggctcctatagcgactaattttttgttcaaactcatgcggttactccttccgtatttttctaaaggtagtgtgggctatggcatccaaaaatatgctttaatttatattgtttttccaagaaggaggaagcatggcgatttataaaaatccaatctggcgctggacgatcaatcttctgtatcctgcgatcatttttattttccagagctgggggccaatcttagattcatgggtttttcctatattgtttgctgctttgttttgtttcttatggagtgatgtgaaggatatgctggcttctacagtgttaacatggggagtagccattccaatatggtggtattttatcgagcgtccaaagcctacattcggtgcagaacattttgccgctcatctatggttaattgtgctgatgtatgtg

SEQ ID NO: 53 - DNA, 485 bp, Paenibacillus sp.
  Marker nucleic acid sequence
gcgattgtgggtcgatgacctgcgcgctttcgaacgcatttgcccggaaatcgacatcggactcgatcagcaatggcagcagggcgtcgaagtccggcactggtcagccgagtggcaggacgcggaggcggccgacgtggtgatcgctgcgtttgcctgccagttgccgggcgtctacatggacgccatggccgcacggggaaagccgccgttgtggatgaacctcgactacctcagcgcggaagactgggtagtcggttgccacggattgccgtcggtgaagtacaagtcggtgcagaagtactttttctttccggggttccagccgggcaccggtggcctgttgcgtgaaaacggtttgctggagcggcgtcagcagtttcagcaggatcccgaggctcagcgacaattcctgcacggtctggggatcgttcctgcagccgatgcgcaactgatctcgctatttgcctacgagaatgccggac

SEQ ID NO: 54 - DNA, 484 bp, Paenibacillus sp.
  Marker nucleic acid sequence
atattgccaggaagaaagcggacatgaagatagacgtcatttcgctggcgttcgggtagaggcaaagtctgttcatggttttctttttttaatccttgtcggagttgctctccggtgcgactgaggatccaagtaactttaacagtaggcccctcaattctcttgcagcacattacgccgccccgactcatcgcgaaaagattacccccccaaaaatcattgatccaaaaactgagaattggcctttccatatgattttcggaggtcagcatcgccccaggcgtacgaaactggttgtaaaggtacgaacccaggaaaggcagcagtaaaacaagcatcccaatgaccaggttgcggcgacgccagattttttgccgcttcatgttcatagccacagtcatactgctagattccttcctgatgttgaatattgctcagtcggaagtcgctttgaggaacttcctttttcgtcttttacttgaga

SEQ ID NO: 55 - DNA, 480 bp, Paenibacillus sp.
  Marker nucleic acid sequence
gatatcgatccgccgctcctccagccactgaatgacctgccgatccgggccttcatcgatgtgcacttcgatccccggatgcgcctcgcgatactgctgcagaatcttcggcaacaacttgatcgaggacgtcggtccgaacgagccgatgcgcagcgtgccgcgtttcatgccgcgcgcatccgctgcctcttggcgcaacgtattggccaaaccaagcatcgcgcgcgcccgcagcagcaattgttcgccgatgtcgctcagttccacttgcgattggtgccggcgaagcagctcgacgcccagttcctgttccagcgacttcaaggcgtgggacaccgctgactgggaaatccccagacgatgggcggcgagggtgaaaccgcgcagttcggcgaccagcgagaagatttcgagttgggtcagggtcatgagtaaagactcattttacgatgatgaggaatcaatcgaatgatacgt

SEQ ID NO: 56 - DNA, 476 bp, Rahnella sp.
  Marker nucleic acid sequence
gcctgtttctttccacctaacccacagttattgacaggactccgaggcgcgccggaggcgctttttaaggtcaaaacctcaacgtcaacggcggaagaaacgatgcgccattgagttgtacgggtttcataaacgcgggagtcgcccaggtgaggcgcgaaaatacccacaacttttttcacttcctcatcgtatgtgttcagctcatcagccacgcggcgggctacacgcacagtctgatcgtctcgtgggacatttgcgccgccctgggctgacatgtacgccataaaatcaccggcatcagcagcagcgcgaacggcttccacttcttcgtcaaaggtttcagtcagattgatggaacggatgcggcggcactcacggtatgaccccatggtaggcaggccgataggatgaaattgagggatccgccaggtagcagcccaggcggtgacagcggcagcggaatctgtcagcag

SEQ ID NO: 57 - DNA, 492 bp, Pseudomonas sp.
  Marker nucleic acid sequence
ctccaggcgatccgctatataaccattagtcgtgtttgctctttcttccattcggatcaggacttcgcgggcaatcgtgctatcggacgaaagcccatttcctcagatatgtctgtcaatcctcagctgtcagtgcttgtaattaggtagcgacagtcgtctggtttaaaaggtttctgttggcctcaaaagacatatgggtgaaattgcttgtcggtggtagggcgttaagttaaatatcaatatagtgaataattattttaatgaactgtctgtcaatttcataacaaataattcactataaattgacaattcagaagttgcaaaaagagcttgccatgagccgaaaaatgctgcatcttttgtaaaaattcttaatccgatgcgtcactttaagttagtgaggtaatgtttgagccagatttatgagttaaagttatgactatactttcatcaactaagtacattagtcgttatcccgttttgatgttg

SEQ ID NO: 58 - DNA, 482 bp, Pseudomonas sp.
  Marker nucleic acid sequence
tgcggagaaaacagggccggcgccagtggtagcctgctcgacagcttgggctcccgctgtgactccgacgctccgatcgagcagaggggagagggtcttcagaactgcctggcgagtcttcacgctcatcccggcgtaaggcccattctcccagtcagacacatcacgctgtactgcctggagttgttgggtgtcgttgcccgcgttatgtatccgggcaagggcattggctgagtaagctgtttgctcaaacgtccggagttccttatcccaaccgtcgacaccataggcagtgcgcccgaccgcctggacgccttcaacacgggcgcgtttcaggatcgtgttcaggtctgcgtctggccttgctgcagcgccttccaggccgtcaaatcgagagcgcagctcactctttgcttggcctgttcttgcctcagttgccgcctgttgtacctgttgaagcttgttcatctgcagtttgcgca

SEQ ID NO: 59 - DNA, 482 bp, Pseudomonas sp.
  Marker nucleic acid sequence
agcaagctcatgcagctcggtaagctcggggtagttaagcttagcatttgcaagtctggcaatcgctttgtcatgatttccagagctccgcagcctctcgatcagcgcaagatcgatgcaagcctctaatctgcgtggcgcatttatccctgtagccttgtatcgctgtttgaagtagttatcatgctgagtaacatgctcttcaatcggccattcggtgttttctccagttgcagcatgaatgactaaagattcaatggaaggcgcatttattgcttcaaggtcgcggctgttaattttggctttaggatggtgagtagggtgggtaagccgagtgaaggcgatgtagaggcccactagagccctatgttgatccgctttcaattggctgcgcttggctaaccctatttctagaacgtcacgaacatcataaagcttgccgtttgggtagtcggccaaatactgatctacaagagaaacat

SEQ ID NO: 60 - DNA, 469 bp, Pseudomonas sp.
  Marker nucleic acid sequence
cggagtacttggcataggccttggccgatgaacgaaggtcccggcggtcgattggcccaaatgaaggcaggggcggtaggccgagcacctcctcgcggatagccaggaccaggccccagcagtcgagcaggtatcgaccggccacgacctcgccccgcgccccgtcttcatacgtcgcggtgaaatatttgtcgtacatggctactggtaggtcagtgctggagcgaaatcggtggtgtagcggaacctgttccacatcatgttgatcaggtcgtaatagccggcctcgatctggatcgtaggtccatccatggtcccgccgaacgatgtcatccggtatggcctgtcagcgggctcgccgggaatgctgtacaggtactcgcggtaaacgatggtgaccctggcctcggcttcgagtgatgcatcgatcagttgctgcgcttcacccgttacgttgtcgataacgaag

SEQ ID NO: 61 - DNA, 466 bp, Pseudomonas sp.
  Marker nucleic acid sequence
cgtctggcttgccgagcgatgcgagtaaagatggcgataggcatatttggcgttggctcgctgctgcccaacgatctgccccagaagcgtcccactgagtccttacacgagagtctggtaagccacaccctgcagtgaaatcgattttcacacccaaggagaatgatgatggttgctcgcaagacagttgaagatctgttcatccatgagctttcggatgtgtacagcgctgagaagcagatcaccaaagcattgccgcgcctggctcgggcatctaccaacccactgttggctgatgcgtttacctcacacctggcggaaacccaggggcagatagagcgcattgaccagttggtggagtcggctggattgaaactcaagcggatgaagtgcgtagccatggagggtctggttgaggaaagcaaagagctgctggatgaaatcgagaagggggcagtgctcgatg

SEQ ID NO: 62 - DNA, 497 bp, Pseudoxanthomonas sp.
  Marker nucleic acid sequence
gttctatattatttccctgctgagaatggcgccatttaggtcgctgatttgcagtatggctcagatcaggcattgggccttgatatgatcgagtcccactattggcgtgccgagttgaggagtgatctggcatggctcaagaagcatcgcagctatcgcagatggtctgagaggcaacaagtcctattcgagcgaaaactgatgctggttgcatttcaggtccgctcgctcctggaaagaccgaaattcaatgatcaggcacgcagcgtcggaatgtcggtgcttcggtatagaaagatcggcaacagcccatttaccgctactggttcggaatggccgtacgagcgcttcaatatggaggaccccgaatcggatgttctgtccgtctttgacgtatgcaaccagctcattcactactattggatgcaaacgtggtcggaaaggcaggcctttgtttccatactcgtcttctcggattacaagcggcacaaatgtgc

SEQ ID NO: 63 - DNA, 482 bp, Pseudoxanthomonas sp.
  Marker nucleic acid sequence
ggggcacagcgtctcaatccagcatgaggacggcgacagctttctggaaaacccattgctcaagaagtacccggacaaactgacgcagctctggagcggcaagcctcccattaagcacaaagttcgtggcgtgtcactcgaggccaaagacgcgcgctgctcgttcgggatgcggattcaacccaagatcttttaccccttcctcatcagaaccgatagaaagaccttcgttctgggtttgtggccgcgcgccattgcggtctgctatgacccggcgaagtttgatgagccgagcattgtgcttcctctaccatcgggtgtgcccggcctgaacccgctccacgaccgattgagagtactgcttgcgcgagcccgcgaaaggtccagcaatggtgaggtcgcacgccaaccgttagcgctggacgattctgcggccgcgttcatggttgagcttaagtaccgcatcaagcaatggggatcgg

SEQ ID NO: 64 - DNA, 481 bp, Pseudoxanthomonas sp.
  Marker nucleic acid sequence
caacatagcgcgatcttggattgcctgataagagcccacgatgcggcatggccatgtatgtttagagcgaaactcagaatcgacttagtttacttgaggcatccctcaacaccccatcggcggcaaaaaccccaccgtcgttacctccacccatttccccttccgcagcacttccaccctcacccgcaccgcgctgatcgccacgaacggcacgaccgtgcccagcgcgtcgccctcccctatgcggggaatccagtcatcggccatcgccggatcgccctgcgccaccgcgtacgcgcgcactgcctgctccagttccgcacgcgtgtagctggggaaacacgcgtgctgttccgctgacggcgtcggcaacacgaacgacaccgcatcgaccgcctccgccacctcccagtccgacacacagtgcttccagtccgtgagatccaggtgcggaccttccgcactcaggcccaggctggtt

SEQ ID NO: 65 - DNA, 481 bp, Pseudoxanthomonas sp.
  Marker nucleic acid sequence
ggcgcctacgtgaacctgggcatcggcctgccgaccctggtcgccaaccacatccccgccggcatggacgtctggctgcagtccgagaacggcctgctcggcatcggccctttcccgaccgaggacgaggtggatgccgacctgatcaatgccggcaagcagactgtcaccgcccgagcgggagccagctacttcggcagccacgactccttcgcgatgatccgtggcggccacatcgacttggcggtgctgggcgccatgcaggtcaccgacaaaggcgacctggccaactggatggtcccgggcaagatggtcaagggcatgggcggcgccatggacctggtggcgggcgtcaagcgcatcgtcgtgctgatggagcacgtggccaaggacggaagccacaagatcctgccgcaatgcgacctgccgctgacgggtgttggtgtggtggacaggatcatcacggacctggcggtttttg

SEQ ID NO: 66 - DNA, 479 bp, Pseudoxanthomonas sp.
  Marker nucleic acid sequence
gcccggcggcaggccggtattgcccaagggatagagcttgccctcgatcttgatgtacaccggcgccccggtggtgagaaacatgtccgaggcgttcttctcggtcatcagcttcaggaaatagccgatgtccatgtgcaaagctcccaagatgcgcggcgcgacgttgcaacggcgccaaaggcttccgacaatggccgcgcgcacccctcaacgtcacggcaccccaatgacagttagcttctcacacttccgccggcccctgcatgtgatggcggccgcagtagcagcactggccgtggccggcgggcctgcattggcccagagcgcattgccccctgaactggaagccgctgtgcaggcagtgcagcgcgcagaccaggccgatgcggaccagtacgccccgcagccgctggaggccgcgagacaggccttggcgcaggcgcaggctgcgcatgcggcacgcgacaagaaggacg

SEQ ID NO: 67 - DNA, 827 bp, Erwinia sp.
  Marker nucleic acid sequence
acggcagctggacgctggacgtgccggcggcggacctggcgggcatcgccgacggtgttgccggcgtgacggcgagcgtggcggacaaggcgggcaacccggcgtcggccagccacaacctgaccgtcgacgtgaccgtgccggtggtgaccatcaacaccgtggcgggcgacgacgtgattaacctggcggagcacggccaggcgcaggttatcagcggctcggcgagcggcgcggcggcgggcgaccgggtgacggtcaccgtcggcgggcagtcgtacaccacggtgctggacgcggcgggcaactggagcgtcgggctgcctgcggcggcggtctccgccctgcaggacggcacggtgaccatcgccgtgtcggtgaccgacgcggccggcaacaccggcggcggcacgcgcgacgtgtcggtcgacaccgggctgccgtcgctgggcttcgacgccatcagcggcgacaacgtgctgaacgcggctgaaaagggtcaggacctgaccgtcagcggcaccagcagcgggctggcggaaaacaccgcggtgacggtgacgctgaacggcaaaacctacgacgcgaccaccgcggcggacggcagctggagcctgacggtgccggcggcggacctggcggcgctgggcgaggcgaactacaccctgagcgccaccgccagcagcggcgtgggcaacagcgtcagcgcgggcgcggggctgcaggtgaataccgccctgccgggcgtgttcatcaacgcggtggccggtgacgacgtgattaacgccgccgagctggcggcgggccagaccctgagcgggcgggta

SEQ ID NO: 68 - DNA, 769 bp, Erwinia sp.
  Marker nucleic acid sequence
gccaggttacccgctccggtgatcagcgcgctgaagttcttcgaggtggcccccgcggtatccagcgccagcgttttgcccgtcgccagcgccagcttaaccagctgcgccgtaacgctcaggccgccgccgatgcccgacaggtcgtagttgtaggtcccggtcgccgccgtgccaccgccctggtcaatcgccacctccacgccggttttaatcgcctggccccgggcatcctgcagcgtcagttttgccagatcttccgcgctcagcgtattgctgctgccgatcagccggcgctgcgtgccgtcatcctgatccagcaggtttccggtggtgacatcgtccggggtcacccggacgatgctgttctgcgccaccgtcagcgtgccgctggtgatgctgctgccgtcggtaaactgcgtggtgccgccgtcaagcgtcaggttgcccaccgcctgcgcgcccgcgcccacggtcaccgacgccccgccgccgatgtcaagcgtcgccgacttcaggctggcggtattgttgccggacaacataaactgcgcattgctcagatttacctgaccgacaaagttgtttccggtgttgtcgccaaagttaaatgccgggtttcctgtcgcagcattaacattcagcgcgccgctcccggtcagcgcctgattaaacgcatttagacccgccagagtcagggcgctgccgtcagcaatatccaccccgatatccgacgcaagattgccggtgctgctcagggtgacatccg

SEQ ID NO: 69 - DNA, 413 bp, Pseudomonas sp.
  Marker nucleic acid sequence
gcggcgatgccggtgtgcacgccagtcttgaagttcagggtgataccggcaccggcgtcggtatacgacacggtatcgttgcccgcgccaccgatgaagcggtcggcaccggcgccaccacgcatcacgtcgttgccactgccaccggtgatgatgttgtcgctggcattgccaaccccggtaaagttgccggtaccaatgtaggtgaggttttccacctcaggggtcagggtgtagctggccagggtcgtgcgcacctcgtcgatgccgccgccagcctgctcgaacacgccagcggtgttgtacaggtaataggtatcgttaccggcaccaccggcaaagctcttgctcgaacctgtcgcctgcatgatgaaggtgtcgttctggctgctgccgatgacactgtacatgcc

SEQ ID NO: 70 - DNA, 389 bp, Pseudomonas sp.
  Marker nucleic acid sequence
ccaaacgcttcgttctggatcgacgttggtcagtccatcgagtcggccaatggcctcgcccagcaagtgcagaccaacaccgccaagatcgaggaggtggacgggaaggtcgaagcatcagccagccagttgagcgcgctgcaagcggcatggcgtccggacagcggggagggagagcttgcggatgcgctcgcgggctggagctccaaggctcgtctggctcgcgagcagctggtccgagcgagcgaaaccgaagcgcttgcgcgcagctccgaaacgctcgacgccaagattggccagaccaacgcaacggtccagaccgtcagccaggcccaggccaacctcgagggtaaggccagcacgatgtggtcggtaaagatgcagctgaa

SEQ ID NO: 71 - DNA, 323 bp, Pseudomonas sp.
  Marker nucleic acid sequence
atgaaggtgtcgttctggctgctgccgatgacactgtacatgcccgtgaacttgtcaccctcggcgtcgccaccctgccccttgccaccggtgttgtcgattgtgatggtcactgcctgctcggacagtgaatagtccatcgtgtcgttaagaccgccctcgaaggtgtgcgcacgagcgtcactaatgaaggtatcggcgtagttcgaaccctggaacagttcgatgctgtcgaaggtatcgccagcggcgatgccggtgtgcacgccagtcttgaagttcagggtgataccggcaccggcgtcggtatacgacacggtatc

SEQ ID NO: 72 - DNA, 601 bp, Pseudomonas sp.
  Marker nucleic acid sequence
ccaccacccgttccatcgctattgaacccgccctcgccccctggctctggaccttgttgctgctggtggtggtctgcctgccacggatcagcatcgacctgtacttgccggcgctgccggccatggccgacgacctgttggccagcgacgcccagctgcaactgaccctgagcctgtacatgctcggctatgccctgtcgatgctgctgggcgggccgctgtgcgaccggtatggccggcgcccggtgctgctggcgggcacggcgctattcctgctggccagcctggcctgcatgctcgccgccaacgtcgaggtgctgatggtcgcccgcctgctgcaagcgttgggcggttgctgcggcaccctgatcggccgggtcatggtccgcgaccgctgcgcccccgaggagcagccgcgattgctcgggctgttgtccatgggcattgccctgtcgccgatgctggcgccgccgctgggcagcctgatcgaccacctgctgggctggcgcggcttgtttctatgcctgagcctggtggccgcggcggcctggctgctgatgtacaagcgccttgccgaaacccgcccgccccagctctgccc

SEQ ID NO: 73 - DNA, 450 bp, Pseudomonas sp.
  Marker nucleic acid sequence
cggcatgcccgaactccggcattgatccgtgggccgccgccaccggccatccatggccgggtggcggctaacccggcgtcctgccgggttacccacggctcaatgcctgcgttcggccatcgtggttaacggggcgttcagatcaagatcaaaaaccagagcaagatcaaaatctgacggcggcctaccggccggcctagtttcatttgtcctttacagggtgacgttgcttttggcaggggcgaaggtcctgcggacctttgcgcagcttcgcaggctcagcagcggctacagaacgcacatactctgtagccgctgccgcaggctgcgaacggctccgcaggagacgcggggatcttaaggccgcccaagatcggcaggcgatcgtcaagcaaggtcctgcggacctttgcgcagcttcgccgtggctcagcagcggctacagggcca

SEQ ID NO: 74 - DNA, 302 bp, Pseudomonas sp.
  Marker nucleic acid sequence
aaagcaagatcaaaatctgacggcggcctgccggccggcctgtttcattgccgttcgcgttaatgtagccgctgccgcaggctgcgaacgctctgggcggcactccgtcgcaggagacgctaaacctgcctccccagttttcctgacataccgcgtcgccctctttcgcagcctgcggcagcggctacaaagagccggcctgcgcaccttggctttggcttttgatcttgctcttgatcttccggccccttcccagccggccgagcgtaggcgttgcgtagggggcagctcggcatggatgc

SEQ ID NO: 75 - DNA, 921 bp, Ensifer sp.
  Marker nucleic acid sequence
tttaccgacatgggcagcctcagcgtgacgatcgccggccagccgctcgaccatctgctttatcacttccggctggtctggtcgggcttcgaacacgcccatgtcatccttggcggtgaaagcttcgtcgccttggcggagggactgcagaatgccctgtggtcggtcggcggttcgccgctctatcaccgcagcgacagcctgtcggctgccttccgcaacctcgacgccgatgccaaggtcgatctgacaaaccgctatgaagacctatgcgctcattaccggatgacgccgacgcgcaacaacaagggcgtcgctcacgagaacggctcaatcgaaagttcccacggccatctcaagaacgcagtccgcgatgcactcctgatgcgcggcacccgagacttcgacgatctccgctcctaccgcgccttcatcgacgagatcgtcagccgccgcaatgccgcacatggcaagcgcatcgatgccgagcgcccgcatctgcaggcacttcccgagcgccgcaccaccgacttcgaggaggtggtcgtcaccgtgtcgcgcaccggcggcttcgccttgcgcaaggtcttctacaccgtcccctcccgtttgatcggccaccggctgcgtgtgcgtttgttcgacgatcgcctcgacgtcttcgtcggcggcacgcatctgctgacgttgccacgcggtcgcgccgacgcgagcggcaggcatgaccaggtcgtcaactatcaccacgtcatccactctctgcgcaaaaagcccatggcgcttctcaacctggtctatcgcgacaagctctttccccggccggaatatcgaagggctttcgacgctctcatcgagcaactgcctgaccggcaggcttgcaagatcaccgtcgaattgctggccctggcgcatgatcgcggc

SEQ ID NO: 76 - DNA, 911 bp, Ensifer sp.
  Marker nucleic acid sequence
atcggttcggcagcctatcttcggtcacttactcctggcggggtacagataccggaattggtcgtgttcaccaccgtgggggtcgcaatttccttcttgggagaaatgctccgacgcgctcaacatgagttaacaaccgctgacgaagccttgcgtactagagaggcgcatctccggtcgatcctggacactgttctcgacgccaccatcgtcagcgagcaggacggcacgatcgtatcgttcaacgctgcggccatcagacagtttggctaccgcgaagatgaggtgatcggccaaaatctgcggatgctgatgccgcaaccctatcgcaaggaacacgacggctatctcgagcggtacctctcgaccggtgaaaaacgtattatcggaaccgaccgtgtggtcgtcggtcaacgaaaggacgggtcgacctttcccatgaaacttgccgtcggggagatgagatccggtggtcgaaggtttttcacaggttttatacgcgacctgactgagcgcgaagaatcggccgctcgtctgcaagaggttcagacggagttggctcgcctcgcccgtctcaacgaaatgggcgaaatggcctcgacgctcgcccacgaattgaaccagccactctccgctatagccaattatgtccacggttgcgggcggctgcttcagcaagccgaaagtccgcgcgacatccagatacgggatgcgctgaaggaagccggcgaacagagcctgcgtgcgggacaaatcatcaaacacttgcgggaattcgtcaccaagggagagacggataagtctccccacaacctgcggcaactggtcgaagaagccggcgcgcttgctttggtcgggtcgcgtgagaaaggcgtgcgaacggtcttcgatttcgaatcaggcgcagacca

SEQ ID NO: 77 - DNA, 863 bp, Ensifer sp.
  Marker nucleic acid sequence
catgcacagcgtccggaagagatcgttttcggcaactcgaccaccgcactgctgcaaaacctcgcccgcgccatgcacagccagctgacggccggcgacgagatcatcatcactgtatcggatcacgaatccaacatcggtccctgggaccggttgcaggaacgcggcgtcatcttgaagatctggccgctgaacaaggaaactttgtcactcgacctcgccgatctcgcccccttgatgagcgagcgcacgaagctcgtctgtgtgactcattgctcgaacatcctcggctccatcaatccgatccgcgagatcgccgatttcgtccatgcccgtggcgccaagatctgcgttgatgccgtcgcctatgccccgcaccgggcggtcgacgtgcaggccttcgacgtcgactattacgtcttcagcctctacaagacctatggtccgcattacgcgctgatgtacggcaaatacgacctgctgctcgagctcgacacgctctaccactacttctacggccgggacaaagtgccgggcaaactggagccgggcaatcccaattacgaactcgcctattcgacctgtggcatcgtcgactatctcgtttcactcggcgaacaggccggcgaaaccggaactgtcaggcagaagatcgaagctgcctatggcgcgatcaccgctcaggaagacgcgctcaccgagcggcttctgacctatcttcgttcgcgcaacgactgccggatcatcgggcagtcggcgaaccccgacagccaacgtgtgcccaccattgccttccgcttcgatggacgcgaggcggcagacctctgcaaggccatggacgccgagaagatcgccatgcgctt

SEQ ID NO: 78 - DNA, 695 bp, Pantoea sp.
  Marker nucleic acid sequence
gttttcagcagcgtgctgccgctcagcaccgagttgtcggcgtcgatattcatgctggtgccggtcagcacgttgacggcggcgccgtcggtagcggctatgcgggtgttggcgagtttaatgtccgccagcgtggcctgccatttggaaagaatcccggtggcgttctcgcctgtgacgttaatagctgaatcagtcagatgaacgtgattgattaaatcagggttatacacattcatgacgttgattccggtattaataatgccagagtctgttctcgatttttcaatggtgacgttcttcccggtgaacgcacttccgtcctgaacctcaatgccagaatcgccactgcgcgcctgtacagaaacattttcggcattagttattgctgaatatgcagtagtcagggccgcattatttgcatcacttataatttcaacatttttgaggttagtcgtagcactatatcgggtaagaaccccgttagtggcttcattccctgactgaatgcgggtgtcagatatattcattatcgttccactgccggaagataaggtgccattaccactgccggtgacctttacttcagaattagtcacgttaagtaccgatttcgaatctgccgtcagaccattagcacctgcgccgctgaccgcaagcgcactgttatccacattcagtactgaaccttcgcc

SEQ ID NO: 79 - DNA, 516 bp, Pantoea sp.
  Marker nucleic acid sequence
accagataccagccgtgctgatccgcgcccttcaccaggttgtagtcataggcaccggcgacgatacgccctttctgacggaattcgccgttgctggcgccgtccacctgaatcagctgcagcccctgaatggtctgcgcgccgctgccgccggcgttgttgaccaccacgtcggtgctgccgctgctgtcgccaaggatgtgcagcttatcgctggcggagtgatcgtcgcccagcgccgcgttcatcaccagcgtgctgttgtcgccgctgtagttgccgttcaccgtcagggtgctgaagccggcgccgtcgtgggtgaagttcagcgtgctgttgctggcactcagcgcatcaacgctgctgtcgccggtaatgttccactggctgttgtcattcagcgtcagcgtattcaggccggatgacgcgccggtccacgccgagctgccgctcaggttgatctggctgtcctgcgtctggctgcggtcgatgtccacgtcaccgatgaacagcgag

SEQ ID NO: 80 - DNA, 455 bp, Pantoea sp.
  Marker nucleic acid sequence
tagcccgcgaacgcctcgtgctggtccgccccgtcgcacagcaccatcacgtgacgctccgcctcgtggcggaaccagtagtaaatcccctccagctccatcagccggctgatgaagtcgaagctgctctcctggtactgcacgcagtactcccacacccggtagctgctgcccagccgcgtctccacgttcaccccgttctcctgcagcagcgtctgcactatctgcggcaccgtctgaccctggaatatccgcaggttgcggtcccggcgcatcggccacaggtccggctccacccgcagctggtacaccgcgtagcgcgtcccgctcagctcctcgctgcgcaccgccacccgcgtgattttcccgttgaggtagcgcggcgacagcaggctctgcgtcggcagcgtgaacgtgaccggctgccccagcagcgcgtgccggtcgatgcgcgc

SEQ ID NO: 81 - DNA, 685 bp, Acinetobacter sp.
  Marker nucleic acid sequence
accgaccttaagcgtgggtgacgacactcaagttgaaggcacgaacctggttcacaccgtgagcttaagcaacccatcgagcacggcacaaagctatgcgatcagcttaacgggcaacaccgcgacggcaggcactgacttcgacaacgtcttggccaatgcggtgtttagcaatggcgtgacttacgatgccggcaccggccaaatcaccgtgcctgcaggcgtgaccagcttcaccgtgggctatgcaaccactcaagacaacatctttgaggcggacgaaaccacaaccttaaccatcggcggatcaacgggtacaggtacgatcaccaatgacgatgcggtaccgaccttaagcgtgggtgacgacactcaagttgaaggcacgaacctggttcacaccgtgagcttaagcaacccatcgagcacggcacaaagctatgcgatcagcttaacgggcaacaccgcgacggcaggcactgacttcgacaacgtcttggccaatgcggtgtttagcaatggcgtgacttacgatgccggcaccggccaaatcaccgtgcctgcaggcgtgaccagcttcaccgtgggctatgcaaccactcaagacaacatctttgaggcggacgaaaccacaaccttaaccatcggcggatcaacgggtacaggtacgatcaccaatgacgat

SEQ ID NO: 82 - DNA, 362 bp, Acinetobacter sp.
  Marker nucleic acid sequence
atgggttgcttaagctcacggtgtgaaccaggttcgtgccttcaacttgagtgtcgtcacccacgcttaaggttggtgctgctgaaacgttgatggttccagtcgcagttgcgctgccgccttggccatcgctgatgctgtagctgaagctttcttgaccgctaaagcctgcgttcggtgtgaaggtgatgttgccttgggcatcgatgttcaccacaccgttcggggtgttgatttgttgggcggtgcccggggtcaaggtcacgccattgatcgcagtaatgatcagtgggttgccatctgcatcggtatcatttgccaatgggttgagggtcactggcgtgttgaatgcggtgttgtac

SEQ ID NO: 83 - DNA, 284 bp, Acinetobacter sp.
  Marker nucleic acid sequence
ggccaagcacacgagggtagccttttgtttgtttgcaatctctgcttttgatggctcaaacaaggagaccttgggtggagtcacatttctcagatcctctaggacggtgagtcgtgtccctggtccgaagaactgctcagcatagttacacagcagaaaagggctaccaagaattcttcctctttcctcaccttcctcaatacagacccggggttactcacattctccatctacctccagaactccccatataagaaatatcatgcaggacaacgggggtaata

SEQ ID NO: 84 - DNA, 203 bp, Enterobacter sp.
  Marker nucleic acid sequence
ctgcggatatcgcgcaccaccaccgtgccgccgccgatgcgcacccggtgatcctccgtcacgctgattttcgcctcgcaggtgctgcgatcgccgtggcgggcggcgggctggccgttgataaaaactttggtggagccttcggccagaaacagcgggccgggtgcgaggtgccatttactgcacgtaatctggtcctgcgg

SEQ ID NO: 85 - DNA, 200 bp, Enterobacter sp.
  Marker nucleic acid sequence
agcggtacggctgaagcgggcagcattgtgtctgtttacgacggcaccacgctgctgggtacggtcactgttggcagcgacggtacctggagcttcaccaccccagccctgaccgatggcgcacacagcctgaccaccaccgtgaccgatgcggcgggtaacgtcagcccggcgagcgatccgatcagcttcaccgtgga

SEQ ID NO: 86 - DNA, 896 bp, Pseudomonas sp.
  Marker nucleic acid sequence
tcggtttggctggagcggtagatgatggtgttggccttggtcacgcgcgatctttgctgagtgaaggtgcgccactcactgcgtagcggtttgccggctgggcagcggtattcattggcctccggactccagaggaagtcgttactggagaggctgtcgtccttgcgctcggtcttgtcccacaccggcacatgcggctcgatgttcttttcttcgaccatccaggccagcatcggggcagtgccgtaggcggtgtccccgatgaggcgttccggcgtgaggtcgaactgcgcttcgacacgctccaccatcgtcctggtcgaatcgacttcggcggtacggtgcgccggagtcgcctccacatccatgatcacgccgtactcagtatcgatcaggtaattcgtggagtaggcaaagaaggccgggccgcctggtgcagcagtccaacgggacagcggatcagtgagcgagattttcttgggaagagcttcagccagcgcctcttcatcaagggcttcaaggtactcgcgcacagcgcggttgctgagcgctggatcacgccaatcgacttcatctcccgccaccccacgttgccggctggcatccgccttgatgatgctggcgtccacagcgaagccttcgcctttgaccaggccagccgccatgcagcggcgcagtacctcattgaacaaccagcgaaacagtccgctgtcacggaagcggccgtggcgattcttcgaaaaggtcgagtgattgggtacttcgtcttccaggcccagtcggcagaaccagcgataggccagatttaaatgcacttcttcgcacagccgtcgctcggaacggatgccgtagcagtagccaacgaccagcatgcgcaccatcaactctgggtcgat

SEQ ID NO: 87 - DNA, 766 bp, Pseudomonas sp.
  Marker nucleic acid sequence
tggctaaagaaaaatttgaacgtaacaaaccgcacgtcaacgttggcaccattggtcacgttgaccatggcaaaaccactctgaccgctgctctgaccaaagtctgctccgagacctggggtggttctgctcgtgcgttcgatcagatcgacaacgcgccggaagagaaagctcgcggtatcaccatcaacacctcccacgttgagtacgactcggcgatccgtcactacgctcacgttgactgccctggtcacgctgactatgtgaagaacatgatcaccggtgctgctcagatggacggcgcgatcctggtttgctccgctgctgacggccccatgccgcagacccgcgagcacattctgctgtcccgtcaggtaggcgttccttacatcgtcgtgttcctgaacaaggccgacatggttgacgacgccgagctgctggaactggtcgaaatggaagttcgcgatctgctgaacacctacgacttcccgggcgacgacactccgatcgttatcggttccgcgctgatggcgctggaaggcaaggacgacaacgaaatcggcgtttccgctgttcgtaagctggtagagactctggatacctacattccggagccggttcgtgccatcgaccagccgttcctgatgccgatcgaagacgtgttctccatctccggccgcggtaccgtggttaccggtcgtgttgagcgcggcatcgtcaaggtccaggaagaagtggaaattgttggtatccgtgatacctccaa

SEQ ID NO: 88 - DNA, 747 bp, Pseudomonas sp.
  Marker nucleic acid sequence
gccctcgatgtttgagccccagggcgggccgttgtcggccgttatccggcatggcagcccatagcgacggaaaaccgcggccagcttggggcgaaccagttccaggcgctccccttcacagccctccaggcagagcgcaaagcgtgaatggtcatcgagcaccgtcagcgggtgacatcgtggcgagttacggtccgaaagatggaagttgcccttgaagtcgatctgccagagctcgttgggttgcttgtgctcgaagcgttgggtggccggtgcctgggcgtcttcggcgtgatagcgcacctggcagccatggcgccggagaatcgcatcgagggtgctgtgatgagggcgatccatagcctttggcagcaggtgaagcagcttgcgagcgccccaataagggaattggtgatgcaggcgcaccacctctcgctccagctcggcgctgctgcgtccaggactcgtcagcggacgacgggactgatcctgcaggccggaatcccccagttcttgatatcgctccagccatttgtaagccgtcttggggctgatgccgtagcgtcgacagagctcccggatgttgctctgcgggtgttgggccaacagtacgaattcacgtcgtatcgacatggtggtgcactcctgccacggcatcgctcgaactcctcttcgaaggattccgtaagcctaaatgcgttacctatgtctcccgacacctgtcacctatgtcccccggctgaacac

SEQ ID NO: 89 - DNA, 292 bp, Acinetobacter sp.
  Marker nucleic acid sequence
tgaaatttaaatgaaacctatcagcggattgacgagatccattatatcaaaatagaagaaaatatatatgatgcgtatgatacgcatcatataaatttgtaatatattgttttataagtttattattaaaaatactatggttttagtaaccatcatgagtaccacctctactttaagttcccaacagctcctttttctttagccagttttaaataaggggtcgtctcaggaaacggaaaatatagcacgctaaggattttcactccgacttcttttaacgatatccaacacg

SEQ ID NO: 90 - DNA, 286 bp, Acinetobacter sp.
  Marker nucleic acid sequence
gtcgccgataagttgtgtaagtactttaactgtacgatgaatgatttgtttgagtttaaggaagaagttgaagaaaaagattaacttaatacttcgtttgataagtttttcgaagaaaaagactagccaatacttagtttgataagtttttctctgcagatctaatattgaattttataaaatatcattacaaacagatactaatttcatggcactgttgcaaataaccacgaatggtaagctgaagactgttttagctaaaggtgcagcatatgaatcctttcca

SEQ ID NO: 91 - DNA, 206 bp, Acinetobacter sp.
  Marker nucleic acid sequence
cacttgtttttaagtggggactaaattaaggcttataggatgaattcataaggtgtgttcagcggacgcttacaattaactgacgaacaaattactcaactcctaatcccgaaacgctttataccacccactccacctgaacatgaaggcaagaacatggtttatgtttttgatagtgaggataagtttgagctgacttatgatga

SEQ ID NO: 92 - DNA, 726 bp, Bacillus sp.
  Marker nucleic acid sequence
cagaatcgtgacgccttctcttccagtttctccgatcccgggggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttg

SEQ ID NO: 93 - DNA, 670 bp, Bacillus sp.
  Marker nucleic acid sequence
ttaatggttgtcctaaatcttgtttcagttccgccatttctaacgaactgtacgttgagcagtgttcttgcagcaagttttcttcttctagtgttagctcttgccctaaatcttgctttaactggatcatttcaagctgctgatccagagaaacctctgctttttgtgcacactcttcttgctcctgttcagcttttaaccaatctggaactagttctttgcggataatccgttttggtgtagctgatgttgttgtttgactttcgttatgtatagcttcttgttggtttgcgtatactgatgaataaccctggtctttaggctttgctaattcatagaattgattttttgcaatctgatagattaatcgacacatatcccgtagggataaagggtttccttgttgattggcttcatggacatatttatctaatcccacaatggacgtatacgcaatttcatgtatgtgctctttctcaatcctaatctctggatctttttttactgcttttgcagcattttgaatgcgagcataaaagtcttgtagttgatgtccatatttacctgcatagacttgattgactttcttaggtacacctttgatatagctgaatgactgtgaaacagatattaaatgattatttatatgatttacaggtaaatcaaggtt

SEQ ID NO: 94 - DNA, 656 bp, Bacillus sp.
  Marker nucleic acid sequence
atgtctgacttagatgcttgttcttctgtattcccttttagtagattggtaaattgattgtaagcaatttgataaacgagacgacacatttcatccagtgatagggtagtacccttatgattagcctcatgcacatatttatctaagcctacaatcgctgtataagcgacttcatgaacttgttctttgacaatctcaatctttgtatctcgtttcactgcttttgcagcattttgaatacgcgtataaaagtcttttagttgatgaccatatttacctgcatacacattatttactttcttaggcacacctttgatataagcaaatgactgagaagcagatattaaatgattatttaaatgatttttaggtaaatcaaggttaataaagggggacgacgctttttcgtcctcaaagcttggtgtatcaacgttttcagcacattctggacttgtcacaggagttgtcacaggagttgtcatttcttcttgttgaagcgttttgattcctgttgtgtcatggtcaaaaaagatttcatcacaaatttgatcaaagttcttatggcttttaaggataaacacatagctatcaggatgagcacctgttgtatcttctttgatataacgtgccacgatcataaacggtgtttcctttaa

SEQ ID NO: 95 - DNA, 876 bp, Streptomyces sp.
  Marker nucleic acid sequence
ggctcgggcagcggctccaccgggcgcagcagctctcccggcacgccgaggggttcgcggctggtggcgaggacggtcaggttcgggcagcgggcgagcagttcctcggcgaggcgggcggcggcgtcgacgacgtgctcgcagttgtccaggacgatcagcatgcggcgccggccgcagtgctcgacgaggcgctcgacggcggtggcctgccggtcggagacggcgacggcccgcatctcctcggcgccggcgccgtacagcacggtctcgcgggcgcccagcgcggtcagcacggcctccggtacggcgccggggtcgtcgacgggggcgagttcggcgagccaggcgccgtcccgcgccggctctccggcgccctcggcggcctcctgtgacagccgggtcttgccggcgccgccgggtccgagcagcgtcaccagccgcgcggccgccagatcgccccgcagcgcctccaggtcggcgtcccgcccgacgaaggaggtgagccgggcccggaggttgccccggggacgggacacggccacgctttcggggccaccggaggcggcggacgcctcgcggtccggatacgcggcggcaccggcggacgcctcgcggtgggagtaccccgcggcgttcacggacaccgaggggtcaggcgctccggtaccggcggatgccacgcggtccggatacgcggcgggaccggcggacgccgacggacgggacgccccgggaccggcggacgcctcaccgcccggatacgcggcgggaccggcggacgccggcgggtaggcgccgccggggtcggcaggtcccccggggtggggcgctcccgggtcggcggtgtcgccgggacgggcggtcgtcgccggg

SEQ ID NO: 96 - DNA, 767 bp, Streptomyces sp.
  Marker nucleic acid sequence
ccggtgaggcgggcctgtcccggatctcgacggcccgcgtcacctcgttgatgctggacttctgcttcatcgcccaggcgatggccttgtcgtgctgcatcacccgcagcaggacgaaggtgtccccggtgggcggcgcgagcaccacaccgcgccagaactggtcgatgcggatcgtccggatctgcttgtccttcgccttcacgaggctttccagcttcaggccctgatccttgaagagctggtcgagggtgagctgattgaacttctcccaggcgtcgaggactcccttccttaccgggggctggagcttgggaagctcggcgaagaaaccgatgtcgaaggcgagctgcggcatgcggagatcctagtgcccgcgcacccctcggtgcgggcgaaggccaggtcagggcagggaaccggcactgtcgccgtcgagatccagacggatcaccgccagggtctcggccacctcggcggtcagctcgtcgtccgggccgaacaccaggcacaggtccgcgtgcagcggctccaggacgtcgaaggcctccgccgcccgctgttgcgcgagcagcagcatgccgatgtcgcgacgcaactcgacggcgttctcgctcacgtcgccatcgacggcccgcaccgcgtcgagcacgcccgtcagcccggccagcgcctcggtcacctggccgagttcgccgcggcagcgcgccgcctgggcccggcactccagcgcttcctcactgctcggaccgctcacgcgcccgtag

SEQ ID NO: 97 - DNA, 760 bp, Streptomyces sp.
  Marker nucleic acid sequence
ggcagcgctcagttcacccaccgaatgcccggccaccatgtcggggcgcacaccccacgactccagcagtcggtagagagccacctcgtgggcgaagagggccggctgtgtgaaccctgtccggtccaggccctcgccgtcccacatcacctcacgcagcggcagcccaagcgcctcgatcacctcgtccagcgcctgcgcgaagaccgggaaagcgttgtacagctcgcgtcccattccggcacgttggctgccctgtccggagaacacgaacgccgtcctgccctgcaccggcgtcccaccggagccgaccgtggcaagcccgctcagcagttcctcgcggtcccggcccacgacgacagcccggtgctcgaacacgctccgggtcgtggccagtgagaatcccacgtccagcggggtcagttcttcccgctctgccacgtgctcggccaggcggtcggcctgggcccgcagggcggcctcgtcacgtcctgacaagacccacggaaccaccggcagcggaacaagagcctccgccgccgcctcttcctcaaccggcggagcctcttccaggatgacgtgggcgttcgtcccgctgataccgaaggacgacacacccgccctgcgcggacgcccgtcgctcgcccactcccgggcctccgtcagaagttctaccgccccttccgaccagtccacgtgcggcgacggctcctccacatgcagcgtgcgcggcagcacgccgtgccgcatcgccatcacc

SEQ ID NO: 98 - DNA, 20 bp, Artificial Sequence
  misc_feature (12)..(12): n is a, c, g, or t
  Primer ## Primer
agagtttgatcntggctcag

SEQ ID NO: 99 - DNA, 22 bp, Artificial Sequence
  misc_feature (6)..(6): n is a, c, g, or t
  Primer ## Primer
tacggntaccttgttacgactt

SEQ ID NO: 100 - DNA, 19 bp, Artificial Sequence
  misc_feature (9)..(9): n is a, c, g, or t
  Primer ## Primer
gtgccagcngccgcggtaa

SEQ ID NO: 101 - DNA, 20 bp, Artificial Sequence
  misc_feature (11)..(11): n is a, c, g, or t
  misc_feature (15)..(15): n is a, c, g, or t
  Primer ## Primer
ccgtcaattcntttnagttt

(19) United States
(12) Patent Application Publication
(10) Pub. No.: US 2024/0228947 A1
(43) Pub. Date: Jul. 11, 2024

(54) BACTERIAL STRAINS HAVING FUNGICIDAL ACTIVITY, COMPOSITIONS COMPRISING SAME AND USE THEREOF

(71) Applicant: LAVIE BIO LTD., Rehovot (IL)

(72) Inventors: Ada Viterbo Fainzilber, Rehovot (IL); Amir Bercovitz, Raanana (IL); Galit Kuznets, Tel Aviv (IL); Anna Movtchan, Ashkelon (IL); Hadar Kimelman, Petach-Tikwa (IL); Michael Ionescu, Mazkeret-Batya (IL)

(21) Appl. No.: 18/559,041

(22) PCT Filed: May 2, 2022
(86) PCT No.: PCT/IL22/50450

§ 371 Date: Nov. 3, 2023

Related U.S. Application Data
(60) Provisional application No. 63/184,791, filed on May 6, 2021.

(51) Int. Cl. C12N 1/20; A01N 63/20; A01N 63/22; A01N 63/25; A01N 63/27; A01N 63/28; A01P 3/00; C12R 1/01; C12R 1/07; C12R 1/18; C12R 1/38; C12R 1/465

(52) U.S. Cl. CPC C12N 1/205; A01N 63/20; A01N 63/22; A01N 63/25; A01N 63/27; A01N 63/28; A01P 3/00; C12R 2001/01; C12R 2001/07; C12R 2001/18; C12R 2001/38; C12R 2001/465

(57) ABSTRACT

The present invention relates to the field of biocontrol of plant pathogenic fungi, particularly to bacterial strains effective in treating and/or preventing plant diseases associated with phytopathogenic fungi and/or oomycetes; preparations, lysates and extracts thereof, compositions comprising same and use thereof.

### FIELD OF THE INVENTION

The present invention relates to the field of biocontrol of plant pathogenic fungi, particularly to bacterial strains effective in treating and/or preventing plant diseases associated with phytopathogenic fungi and/or oomycetes; preparations, lysates and extracts thereof, compositions comprising same and use thereof.

### BACKGROUND OF THE INVENTION

During all developmental stages, plants are exposed to an extremely wide range of biotic and abiotic stress conditions, leading to plant diseases. In the production of crop plants, damages caused by biotic stresses, particularly by pathogenic agents, which may be further enhanced under conditions of abiotic stress, pose a major problem and significantly affect the crop yield and profitability.

Many plant diseases are caused by plant pathogenic fungi and oomycetes, and yearly damages in yield to both monocotyledonous and dicotyledonous crop plants amount to billions of US dollars in the U.S. alone. For example, Fusarium seedling blight symptoms are caused by several Fusarium species, particularly Fusarium oxysporum and Fusarium graminearum, which cause the disease in tomato and corn seedlings, respectively. Fusarium verticillioides causes ear rot disease in corn, particularly when corn plants are exposed to warm dry weather early in the growing season, followed by wet weather during the development of the cob. Botrytis cinerea causes Gray Mold disease in tomato fruits, grapes and many other fruits and berries.

Pythium spp. (P. aphanidermatum, P. irregulare, P. ultimum, and others) are oomycetes that may affect a wide range of crop plants, particularly crops grown in greenhouses, causing pre- and post-emergence damping off, as well as root rot. Pre-emergence damping off occurs when seeds are infected prior to germination. This can result in poor or no germination, and is observable as a browning or rotting of the seed and root. Post-emergence damping off takes place after germination, and results in a thinning, water-soaked stem near the plant collar, which eventually causes the collapse of the plant. Symptoms of root rot include stunted growth, chlorotic leaves, leaf drop, and wilting.

Downy Mildew is one of the most serious grapevine (Vitis vinifera) diseases in the world. It is caused by the biotrophic oomycete Plasmopara viticola, which can attack all green parts of the grapevine. No effective biological solutions are available nowadays against this disease, and chemical treatments afford only partial protection.

Fungal and oomycete pathogens are typically controlled by the use of synthetic chemicals (e.g., fungicides). However, although these chemicals may be effective, synthetic chemicals increase agricultural production costs, and moreover, are typically toxic to animals and humans and have harmful effects on the environment. Additionally, pathogen resistance to such chemicals is rising as a result of overuse. In some countries, certain fungicides or anti-oomycete chemicals have been restricted or banned for these and other reasons. Therefore, there is a growing interest in developing control methods and compositions that do not rely on toxic synthetic chemical fungicides or anti-oomycete compounds, or that reduce the use of such chemicals.

Biocontrol agents are typically microorganisms, such as bacteria or one or more products thereof, that are applied to a plant or a part thereof, or to the plant habitat, to control a pathogen. Biocontrol agents are good candidates to replace toxic fungicides/anti-oomycete compounds. For example, International (PCT) Patent Application Publication No. WO 2014/173906 discloses a novel bacterial strain, Lysobacter capsici, and uses thereof for plant protection, particularly for protecting plants from pathogenic fungi and/or oomycetes. The invention also relates to the combined use of compositions comprising copper (such as copper-containing plant protection products) and Lysobacter capsici bacteria in treating such pathogens.

International (PCT) Patent Application Publication No. WO 2016/156164 discloses strain CECT8836 of Bacillus amyloliquefaciens and mutants thereof, and the use of said strain, extracts thereof and compositions comprising same, as a pesticide in controlling plant diseases caused by fungi and bacteria.

However, there is a limited number of commercially available biocontrol agents. Furthermore, most known biocontrol agents are limited to control of single phytopathogens in their known effect and/or their practical use. For these and other reasons, there remains a need for compositions containing biocontrol agents for the control of a broad spectrum of pathogenic fungi and/or oomycetes.

### SUMMARY OF THE INVENTION

The present invention answers the need for safe biocontrol agents that can protect plants, particularly crop plants, from diseases caused by pathogenic fungi and/or oomycetes. The present invention provides bacterial strains showing unexpectedly effective activity in combating a wide range of phytopathogenic fungi and/or oomycetes and combinations thereof. In certain aspects, the present invention provides a modified bacterial strain having an improved antifungal/antioomycete activity obtained by directed evolution. The present invention further provides preparations of the bacterial strains, lysates, extracts, whole cell broths, compositions comprising same and uses thereof. In certain aspects, the present invention provides methods for conferring or enhancing the resistance of plants towards pathogenic fungi and/or oomycetes, or to protect the plant from a disease caused by the pathogenic fungi and/or oomycetes, comprising contacting the plants, or the plants' immediate surroundings, with the bacterial strain or a combination of bacterial strains.

According to one aspect, the present invention provides an isolated bacterial strain or a functional homolog thereof, wherein the isolated bacterial strain is selected from the group consisting of:
(1) strain LAV58567, the strain being selected from the group consisting of
   a. a strain deposited under Accession Number 43954 at NCIMB;
   b. a strain comprising a 16S-rRNA sequence comprising the nucleic acid sequence set forth in SEQ ID NO:5;
   c. a strain comprising at least one genomic marker comprising the nucleic acid sequence set forth in any one of SEQ ID NOs:36-38;
and any combination thereof,
(2) strain LAV104662, the strain being selected from the group consisting of
   a. a strain deposited under Accession Number 43955 at NCIMB;
   b. a strain comprising a 16S-rRNA sequence comprising the nucleic acid sequence set forth in SEQ ID NO:4;
   c. a strain comprising at least one genomic marker comprising the nucleic acid sequence set forth in any one of SEQ ID NOs:33-35;
   and any combination thereof;
(3) strain LAV104858, the strain being selected from the group consisting of
   a. a strain deposited under Accession Number 43956 at NCIMB;
   b. a strain comprising a 16S-rRNA sequence comprising the nucleic acid sequence set forth in SEQ ID NO:3;
   c. a strain comprising at least one genomic marker comprising the nucleic acid sequence set forth in any one of SEQ ID NOs:30-32;
   and any combination thereof;
(4) strain LAV104632, the strain being selected from the group consisting of
   a. a strain deposited under Accession Number 43957 at NCIMB;
   b. a strain comprising a 16S-rRNA sequence comprising the nucleic acid sequence set forth in SEQ ID NO:2;
   c. a strain comprising at least one genomic marker comprising the nucleic acid sequence set forth in SEQ ID NO:29;
   and any combination thereof;
(5) strain LAV104629, the strain being selected from the group consisting of
   a. a strain deposited under Accession Number 43958 at NCIMB;
   b. a strain comprising a 16S-rRNA sequence comprising the nucleic acid sequence set forth in SEQ ID NO: 1;
   c. a strain comprising at least one genomic marker comprising the nucleic acid sequence set forth in any one of SEQ ID NOs:27-28;
   and any combination thereof;
(6) strain LAV1000636, the strain being selected from the group consisting of
   a. a strain deposited under Accession Number 43964 at NCIMB;
   b. a strain comprising a 16S-rRNA sequence comprising the nucleic acid sequence set forth in SEQ ID NO:6;
   c. a strain comprising at least one genomic marker comprising the nucleic acid sequence set forth in SEQ ID NO:39;
   and any combination thereof;
(7) strain LAV1000506, the strain being selected from the group consisting of
   a. a strain deposited under Accession Number 43965 at NCIMB;
   b. a strain comprising a 16S-rRNA sequence comprising the nucleic acid sequence set forth in SEQ ID NO:7;
   c. a strain comprising at least one genomic marker comprising the nucleic acid sequence set forth in any one of SEQ ID NOs:40-42;
   and any combination thereof;
(8) strain LAV1005101, the strain being selected from the group consisting of:
   a. a strain deposited under Accession Number 43959 at NCIMB;
   b. a strain comprising a 16S-rRNA sequence comprising the nucleic acid sequence set forth in SEQ ID NO: 13;
   c. a strain comprising at least one genomic marker comprising the nucleic acid sequence set forth in any one of SEQ ID NOs:62-66;
   and any combination thereof;
(9) strain LAV1001107, the strain being selected from the group consisting of:
   a. a strain deposited under Accession Number 43960 at NCIMB;
   b. a strain comprising a 16S-rRNA sequence comprising the nucleic acid sequence set forth in SEQ ID NO: 12;
   c. a strain comprising at least one genomic marker comprising the nucleic acid sequence set forth in any one of SEQ ID NOs:57-61;
   and any combination thereof;
(10) strain LAV1001081, the strain being selected from the group consisting of:
   a. a strain deposited under Accession Number 43961 at NCIMB;
   b. a strain comprising a 16S-rRNA sequence comprising the nucleic acid sequence set forth in SEQ ID NO: 11;
   c. a strain comprising at least one genomic marker comprising the nucleic acid sequence set forth in SEQ ID NO:56;
   and any combination thereof;
(11) strain LAV1000847, the strain being selected from the group consisting of:
   a. a strain deposited under Accession Number 43962 at NCIMB;
   b. a strain comprising a 16S-rRNA sequence comprising the nucleic acid sequence set forth in SEQ ID NO:9;
   c. a strain comprising at least one genomic marker comprising the nucleic acid sequence set forth in any one of SEQ ID NOs:48-50;
   and any combination thereof;
(12) strain LAV1000965, the strain being selected from the group consisting of:
   a. a strain deposited under Accession Number 43963 at NCIMB;
   b. a strain comprising a 16S-rRNA sequence comprising the nucleic acid sequence set forth in SEQ ID NO:8;
   c. a strain comprising at least one genomic marker comprising the nucleic acid sequence set forth in any one of SEQ ID NOs:43-47;
   and any combination thereof;
(13) strain LAV1000933, the strain being selected from the group consisting of:
   a. a strain comprising a 16S-rRNA sequence comprising the nucleic acid sequence set forth in SEQ ID NO: 10;
   b. a strain comprising at least one genomic marker comprising the nucleic acid sequence set forth in any one of SEQ ID NOs:51-55;
   and a combination thereof;
(14) strain LAV104661, the strain being selected from the group consisting of
   a. a strain comprising a 16S-rRNA sequence comprising the nucleic acid sequence set forth in SEQ ID NO: 14;
   b. a strain comprising at least one genomic marker comprising the nucleic acid sequence set forth in any one of SEQ ID NOs:67-68;
   and a combination thereof;
(15) strain LAV104665, the strain being selected from the group consisting of
   a. a strain comprising a 16S-rRNA sequence comprising the nucleic acid sequence set forth in SEQ ID NO: 15;
   b. a strain comprising at least one genomic marker comprising the nucleic acid sequence set forth in any one of SEQ ID NOs:69-71;
   and a combination thereof;
(16) strain LAV104794, the strain being selected from the group consisting of
   a. a strain comprising a 16S-rRNA sequence comprising the nucleic acid sequence set forth in SEQ ID NO: 16;
   b. a strain comprising at least one genomic marker comprising the nucleic acid sequence set forth in any one of SEQ ID NOs:72-74;
   and a combination thereof;
(17) strain LAV104618, the strain being selected from the group consisting of
   a. a strain comprising a 16S-rRNA sequence comprising the nucleic acid sequence set forth in SEQ ID NO: 17;
   b. a strain comprising at least one genomic marker comprising the nucleic acid sequence set forth in any one of SEQ ID NOs:75-77;
   and a combination thereof;
(18) strain LAV104692, the strain being selected from the group consisting of
   a. a strain comprising a 16S-rRNA sequence comprising the nucleic acid sequence set forth in SEQ ID NO: 18;
   b. a strain comprising at least one genomic marker comprising the nucleic acid sequence set forth in any one of SEQ ID NOs:78-80;
   and a combination thereof;
(19) strain LAV104891, the strain being selected from the group consisting of
   a. a strain comprising a 16S-rRNA sequence comprising the nucleic acid sequence set forth in SEQ ID NO: 19;
   b. a strain comprising at least one genomic marker comprising the nucleic acid sequence set forth in any one of SEQ ID NOs:81-83;
   and a combination thereof;
(20) strain LAV104630, the strain being selected from the group consisting of
   a. a strain comprising a 16S-rRNA sequence comprising the nucleic acid sequence set forth in SEQ ID NO:20;
   b. a strain comprising at least one genomic marker comprising the nucleic acid sequence set forth in any one of SEQ ID NOs:84-85;
   and a combination thereof;
(21) strain LAV104664, the strain being selected from the group consisting of
   a. a strain comprising a 16S-rRNA sequence comprising the nucleic acid sequence set forth in SEQ ID NO:21;
   b. a strain comprising at least one genomic marker comprising the nucleic acid sequence set forth in any one of SEQ ID NOs:86-88;
   and a combination thereof;
(22) strain LAV104922, the strain being selected from the group consisting of
   a. a strain comprising a 16S-rRNA sequence comprising the nucleic acid sequence set forth in SEQ ID NO:22;
   b. a strain comprising at least one genomic marker comprising the nucleic acid sequence set forth in any one of SEQ ID NOs:89-91;
   and a combination thereof;
(23) strain LAV59524, the strain being selected from the group consisting of
   a. a strain comprising a 16S-rRNA sequence comprising the nucleic acid sequence set forth in SEQ ID NO:23;
   b. a strain comprising at least one genomic marker comprising the nucleic acid sequence set forth in any one of SEQ ID NOs:92-94;
   and a combination thereof;
(24) strain LAV104961, the strain being selected from the group consisting of
   a. a strain comprising a 16S-rRNA sequence comprising the nucleic acid sequence set forth in SEQ ID NO:25;
   b. a strain comprising at least one genomic marker comprising the nucleic acid sequence set forth in any one of SEQ ID NOs:95-97;
   and a combination thereof;
(25) strain LAV104960, the strain comprising a 16S-rRNA sequence comprising the nucleic acid sequence set forth in SEQ ID NO:24; and
(26) strain LAV104533, the strain comprising a 16S-rRNA sequence comprising the nucleic acid sequence set forth in SEQ ID NO:26.

Each possibility represents a separate embodiment of the present invention.

According to certain embodiments, strain LAV58567 is of the genus Lysobacter.

According to certain embodiments, strain LAV104662 is of the genus Sphingobium.

According to certain embodiments, strains LAV104858, LAV1000636, LAV1001107, LAV1000847, LAV1000965, LAV104665, LAV104794, and LAV104664, each is of the genus Pseudomonas.

According to certain embodiments, strains LAV104632 and LAV59524, each is of the genus Bacillus.

According to certain embodiments, strains LAV104629 and LAV104692, each is of the genus Pantoea.

According to certain embodiments, strain LAV1000506 is of the genus Gordonia.

According to certain embodiments, strain LAV1005101 is of the genus Pseudoxanthomonas.

According to certain embodiments, strain LAV1001081 is of the genus Rahnella.

According to certain embodiments, strain LAV1000933 is of the genus Paenibacillus.

According to certain embodiments, strain LAV104661 is of the genus Erwinia.

According to certain embodiments, strain LAV104618 is of the genus Ensifer.

According to certain embodiments, strains LAV104891 and LAV104922, each is of the genus Acinetobacter.

According to certain embodiments, strain LAV105630 is of the genus Enterobacter.

According to certain embodiments, strains LAV104960 and LAV104961, each is of the genus Streptomyces.

According to certain embodiments, strain LAV104533 is of the genus Actinokineospora.

It is to be explicitly understood that the species of a bacterial strain of the invention as described herein is classified to the closest known species according to said species characterization as are known in the art.

According to certain embodiments, the functional homolog of bacterial strain LAV58567 comprises a 16S-rRNA sequence at least 97% identical to SEQ ID NO:5; at least one genomic marker having at least 95% local identity to a nucleic acid sequence set forth in any one of SEQ ID NOs:36-38 over 90% coverage; or a combination thereof.

According to certain embodiments, the functional homolog of bacterial strain LAV104662 comprises a 16S-rRNA sequence at least 97% identical to SEQ ID NO:4; at least one genomic marker having at least 95% local identity to a nucleic acid sequence set forth in any one of SEQ ID NOs:33-35 over 90% coverage; or a combination thereof.

According to certain embodiments, the functional homolog of bacterial strain LAV104858 comprises a 16S-rRNA sequence at least 97.9% identical to SEQ ID NO:3; at least one genomic marker having at least 95% local identity to a nucleic acid sequence set forth in any one of SEQ ID NOs:30-32 over 90% coverage; or a combination thereof.

According to certain embodiments, the functional homolog of bacterial strain LAV104632 comprises a 16S-rRNA sequence at least 97% identical to SEQ ID NO:2; at least one genomic marker having at least 95% local identity to a nucleic acid sequence set forth in SEQ ID NO:29 over 90% coverage; or a combination thereof.

According to certain embodiments, the functional homolog of bacterial strain LAV104629 comprises a 16S-rRNA sequence at least 97% identical to SEQ ID NO:1; at least one genomic marker having at least 95% local identity to a nucleic acid sequence set forth in any one of SEQ ID NOs:27-28 over 90% coverage; or a combination thereof.

According to certain embodiments, the functional homolog of bacterial strain LAV1000636 comprises a 16S-rRNA sequence at least 98.7% identical to SEQ ID NO:6; at least one genomic marker having at least 95% local identity to a nucleic acid sequence set forth in SEQ ID NO:39 over 90% coverage, or a combination thereof.

According to certain embodiments, the functional homolog of bacterial strain LAV1000506 comprises a 16S-rRNA sequence at least 97% identical to SEQ ID NO:7; at least one genomic marker having at least 95% local identity to a nucleic acid sequence set forth in any one of SEQ ID NOs:40-42 over 90% coverage; or a combination thereof.

According to certain embodiments, the functional homolog of bacterial strain LAV1005101 comprises a 16S-rRNA sequence at least 97% identical to SEQ ID NO:13; at least one genomic marker having at least 95% local identity to a nucleic acid sequence set forth in any one of SEQ ID NOs:62-66 over 90% coverage; or a combination thereof.

According to certain embodiments, the functional homolog of bacterial strain LAV10011071 comprises a 16S-rRNA sequence at least 97% identical to SEQ ID NO:12; at least one genomic marker having at least 95% local identity to a nucleic acid sequence set forth in any one of SEQ ID NOs:57-61 over 90% coverage; or a combination thereof.

According to certain embodiments, the functional homolog of bacterial strain LAV1001081 comprises a 16S-rRNA sequence at least 97% identical to SEQ ID NO:11; a genomic marker having at least 95% local identity to the nucleic acid sequence set forth in SEQ ID NO:56 over 90% coverage; or a combination thereof.

According to certain embodiments, the functional homolog of bacterial strain LAV1000847 comprises a 16S-rRNA sequence at least 99.5% identical to SEQ ID NO:9; at least one genomic marker having at least 95% local identity to a nucleic acid sequence set forth in any one of SEQ ID NOs:48-50 over 90% coverage; or a combination thereof.

According to certain embodiments, the functional homolog of bacterial strain LAV1000965 comprises a 16S-rRNA sequence at least 97.6% identical to SEQ ID NO:8; at least one genomic marker having at least 95% local identity to a nucleic acid sequence set forth in any one of SEQ ID NOs:43-47 over 90% coverage; or a combination thereof.

According to certain embodiments, the functional homolog of bacterial strain LAV1000933 comprises a 16S-rRNA sequence at least 97% identical to SEQ ID NO:10; at least one genomic marker having at least 95% local identity to a nucleic acid sequence set forth in any one of SEQ ID NOs:51-55 over 90% coverage; or a combination thereof.

According to certain embodiments, the functional homolog of bacterial strain LAV104661 comprises a 16S-rRNA sequence at least 97% identical to SEQ ID NO:14; at least one genomic marker having at least 95% local identity to a nucleic acid sequence set forth in any one of SEQ ID NOs:67-68 over 90% coverage; or a combination thereof.

According to certain embodiments, the functional homolog of bacterial strain LAV104665 comprises a 16S-rRNA sequence at least 97% identical to SEQ ID NO:15; at least one genomic marker having at least 95% local identity to a nucleic acid sequence set forth in any one of SEQ ID NOs:69-71 over 90% coverage; or a combination thereof.

According to certain embodiments, the functional homolog of bacterial strain LAV104794 comprises a 16S-rRNA sequence at least 97% identical to SEQ ID NO:16; at least one genomic marker having at least 95% local identity to a nucleic acid sequence set forth in any one of SEQ ID NOs:72-74 over 90% coverage; or a combination thereof.

According to certain embodiments, the functional homolog of bacterial strain LAV104618 comprises a 16S-rRNA sequence at least 97.1% identical to SEQ ID NO:17; at least one genomic marker having at least 95% local identity to a nucleic acid sequence set forth in any one of SEQ ID NOs:75-77 over 90% coverage.

According to certain embodiments, the functional homolog of bacterial strain LAV104692 comprises a 16S-rRNA sequence at least 97% identical to SEQ ID NO:18; at least one genomic marker having at least 95% local identity to a nucleic acid sequence set forth in any one of SEQ ID NOs:78-80 over 90% coverage; or a combination thereof.

According to certain embodiments, the functional homolog of bacterial strain LAV104891 comprises a 16S-rRNA sequence at least 97% identical to SEQ ID NO:19; at least one genomic marker having at least 95% local identity to a nucleic acid sequence set forth in any one of SEQ ID NOs:81-83 over 90% coverage; or a combination thereof.

According to certain embodiments, the functional homolog of bacterial strain LAV104630 comprises a 16S-rRNA sequence at least 97% identical to SEQ ID NO:20; at least one genomic marker having at least 95% local identity to a nucleic acid sequence set forth in any one of SEQ ID NOs:84-85 over 90% coverage; or a combination thereof.

According to certain embodiments, the functional homolog of bacterial strain LAV104664 comprises a 16S-rRNA sequence at least 97% identical to SEQ ID NO:21; at least one genomic marker having at least 95% local identity to a nucleic acid sequence set forth in any one of SEQ ID NOs:86-88 over 90% coverage; or a combination thereof.

According to certain embodiments, the functional homolog of bacterial strain LAV104922 comprises a 16S-rRNA sequence at least 97% identical to SEQ ID NO:22; at least one genomic marker having at least 95% local identity to a nucleic acid sequence set forth in any one of SEQ ID NOs:89-91 over 90% coverage; or a combination thereof.

According to certain embodiments, the functional homolog of bacterial strain LAV59524 comprises a 16S-rRNA sequence at least 97% identical to SEQ ID NO:23; at least one genomic marker having at least 95% local identity to a nucleic acid sequence set forth in any one of SEQ ID NOs:92-94 over 90% coverage; or a combination thereof.

According to certain embodiments, the functional homolog of bacterial strain LAV104961 comprises a 16S-rRNA sequence at least 97% identical to SEQ ID NO:25; at least one genomic marker having at least 95% local identity to a nucleic acid sequence set forth in any one of SEQ ID NOs:95-97 over 90% coverage; or a combination thereof.

According to certain embodiments, the functional homolog of bacterial strain LAV104960 comprises a 16S-rRNA sequence at least 97.9% identical to SEQ ID NO:24.

According to certain embodiments, the functional homolog of bacterial strain LAV104533 comprises a 16S-rRNA sequence at least 97% identical to SEQ ID NO:26.

According to certain embodiments, a functional homolog of a bacterial strain of the invention is characterized by having substantially the same coding and/or non-coding sequence orientation as that of the bacterial strain of the invention.

It is to be explicitly understood that the present invention encompasses a bacterium of the bacterial strains or the functional homolog strains thereof, as well as a bacterium derivable from bacterial strains or from the functional homolog strains thereof.

According to certain embodiments, the bacterial strains, functional homolog strains and bacterium derived therefrom are characterized by a capability to inhibit the growth and/or the development and/or the activity of at least one plant pathogenic fungus and/or oomycete.

According to certain exemplary embodiments, the functional homolog and the strain of the invention belong to the same species.

According to certain exemplary embodiments, the isolated strain is LAV58567 or a functional homolog thereof, wherein each of the strain and the functional homolog thereof is effective in inhibiting the growth and/or the development and/or the activity of least one pathogenic fungus and/or oomycete, and/or in protecting a plant from at least one pathogenic fungus and/or oomycete.

According to another aspect, the present invention provides a bacterial preparation comprising a plurality of bacteria of at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, and functional homologs thereof, wherein the bacterial strain or functional homolog thereof is effective in inhibiting the growth and/or the development and/or the activity of least one pathogenic fungus and/or oomycete, and/or in protecting a plant from at least one pathogenic fungus and/or oomycete. Each possibility represents a separate embodiment of the present invention.

According to certain embodiments, the functional homolog is a homolog of strain LAV58567, LAV104662, LAV104632, LAV104629, LAV1000506, LAV1005101, LAV10011071, LAV1001081, LAV1000933, LAV104661, LAV104665, LAV104794, LAV104692, LAV104891, LAV104630, LAV104664, LAV104922, LAV59524, LAV104961, or LAV104533. Each possibility represents a separate embodiment of the present invention.

According to certain embodiments, the bacterial preparation comprises a culture medium. Culture media suitable for the growth of the various bacterial genera and species according to the teachings of the present invention are known to a person skilled in the Art. According to certain embodiments, the bacterial strain or functional homolog thereof is present in the preparation at a concentration which exceeds that found in nature. According to some embodiments, the bacterial preparation comprises additional microbial strains and/or bacterial strains other than the bacterial strains of the invention.

According to certain exemplary embodiments, the bacterial preparation comprises a plurality of bacteria from a single bacterial strain of the invention, wherein said preparation is devoid of other bacterial strains and/or microbial species. According to certain further exemplary embodiments, the preparation comprises a plurality of strains of the present invention, wherein said preparation is devoid of other microbial species.

According to certain embodiments, the bacterial preparation comprises viable bacterial cells (capable of replicating). According to some embodiments, the viable cells are in a dormant form. According to other embodiments, the bacterial preparation comprises non-viable forms of the bacterial strains of the invention.

According to certain additional aspects, the present invention provides a lysate of at least one bacterial strain of the invention or the functional homologs thereof. According to the certain embodiments, the lysate is of a single strain. According to some embodiments, the lysate comprises a whole cell lysate of the bacterial cells. According to some embodiments, the lysate comprises a soluble fraction of the bacterial cells. According to some embodiments, the lysate comprises inclusion bodies of a bacterial preparation.

According to certain further aspects, the present invention provides a cell extract of at least one bacterial strain of the invention or the functional homologs thereof.

According to yet further certain aspects, the present invention provides a whole cell broth collected from fermentation of at least one bacterial strain of the invention or the functional homologs thereof.

According to certain embodiments, the lysate, extract or broth is obtained from a plurality of the bacterial cells. According to certain embodiments, the lysate, extract or broth is of bacterial cells of the same bacterial species and/or strain. According to certain embodiments, the lysate, extract, or broth is of bacterial cells of different species and/or strains.

The bacterial strain and functional homologs thereof are as described hereinabove.

According to a further aspect, the present invention provides an agricultural composition comprising a plurality of bacteria of at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, and functional homologs thereof, a preparation of same, a lysate, a broth or an extract thereof. Each possibility represents a separate embodiment of the present invention. According to certain currently exemplary embodiments, the agricultural composition further comprises an agriculturally acceptable diluent(s) or carrier(s).

According to yet certain additional aspects, the present invention provides use of a plurality of at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, and functional homologs thereof, a preparation of same, a lysate, a broth or an extract thereof, for the production of an agricultural composition. Each possibility represents a separate embodiment of the present invention.

According to certain embodiments, the functional homolog is a homolog of strain LAV58567, LAV104662, LAV104632, LAV104629, LAV1000506, LAV1005101, LAV10011071, LAV1001081, LAV1000933, LAV104661, LAV104665, LAV104794, LAV104692, LAV104891, LAV104630, LAV104664, LAV104922, LAV59524, LAV104961, or LAV104533. Each possibility represents a separate embodiment of the present invention.

According to certain embodiments, the agricultural composition comprises a combination of at least two, at least three, at least four, at least five or more distinct bacterial strains and/or functional homologs thereof. According to some embodiments, the agricultural composition comprises a lysate, an extract or a broth obtained from a combination of at least two, at least three, at least four, at least five or more distinct bacterial strains and/or functional homologs thereof.

According to certain embodiments, the agricultural composition is a plant protection product effective in preventing or treating at least one plant disease caused by a pathogenic fungus and/or oomycete.

According to certain embodiments, the agricultural composition further comprises at least one additional active agent selected from the group consisting of a fertilizer, an acaricide, a bactericide, an additional fungicide, an insecticide, a microbicide, a nematicide, a pesticide, a plant growth regulator, a rodenticide, a nutrient and any combination thereof. Each possibility represents a separate embodiment of the present invention. According to certain embodiments, the at least one additional active agent is a synthetic agent.

The agricultural composition can be formulated in any form suitable for applying the composition to a plant or a part thereof or to the plant habitat as is known in the art. According to certain embodiments, the agricultural composition is formulated in a form selected from the group consisting of an emulsion, a colloid, a dust, a granule, a pellet, a powder, a spray, a pressurized form, a pressurizable form, and a solution. Each possibility represents a separate embodiment of the present invention.

According to certain embodiments, the formulation further comprises at least one of a stabilizer, a tackifier, a preservative, a carrier, a surfactant, and a combination thereof. Each possibility represents a separate embodiment of the present invention.

According to certain embodiments, the formulation is substantially stable for at least 30 days at a temperature range of from about 4° C. to about 37° C. According to certain exemplary embodiments, the formulation is substantially stable at a temperature range of from about 20° C. to 25° C. for more than 30 days. According to certain additional exemplary embodiments, the formulation is substantially stable at a temperature range of from about 2-8° C., typically at 4° C., for at least 30 days.

According to some embodiments, the formulation is in a form selected from the group consisting of liquid, solid, semi-solid, gel or powder. Each possibility represents a separate embodiment of the present invention.

According to certain embodiments, the carrier is a plant seed. According to these embodiments, the present invention provides an agricultural composition comprising at least one plant seed and at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, LAV100930, and functional homologs thereof, a preparation of same, a lysate, a broth or an extract thereof. Each possibility represents a separate embodiment of the present invention.

According to certain exemplary embodiments, the strain is LAV58567 and/or a functional homolog thereof.

According to certain currently exemplary embodiments, the agricultural composition is in a form of seed coating. According to these embodiments, the seed coating formulation further comprises at least one agent selected from the group consisting of a binding agent and a wetting agent. According to certain exemplary embodiments, the binding agent is carboxymethyl cellulose (CMC).

According to yet additional certain aspects, the present invention provides a seed coated with an agricultural composition comprising at least one bacterial strain, a lysate, an extract or a broth thereof as described herein.

According to certain aspects, the present invention provides a container adapted for a watering system of a plant field, comprising an agricultural composition comprising at least one bacterial strain, a lysate, an extract or a broth thereof as described herein.

According to certain aspects, the present invention provides a kit comprising (i) an agricultural composition comprising at least one bacterial strain, a lysate, an extract or a broth thereof as described herein, optionally (ii) a delivery system for applying the agricultural composition to a plant or a part thereof or to the plant growth medium and (iii) instructions for using the agricultural composition.

According to certain embodiments, the instructions for using the agricultural composition comprise instructions for the amounts and frequency of applying the agricultural composition so as to confer or enhance the resistance of the plant towards pathogenic fungi and/or oomycetes or to protect the plant from a disease caused by the pathogenic fungi and/or oomycetes.

According to a further aspect, the present invention provides a method for enhancing and/or conferring resistance of a plant or a part thereof towards at least one disease caused by a phytopathogenic fungus and/or oomycete, comprising contacting the plant, part thereof or the plant habitat with at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, and functional homologs thereof, a preparation of same, a lysate thereof, an extract thereof, a broth obtained therefrom or a composition comprising same. Each possibility represents a separate embodiment of the present invention.

According to certain exemplary embodiments, the bacterial strain is LAV58567 and/or a functional homolog thereof.

According to certain embodiments, the plant is susceptible to the at least one disease caused by the phytopathogenic fungus and/or oomycete. According to some embodiments, the method further comprises identifying a plant to be susceptible to the at least one disease caused by the phytopathogenic fungus and/or oomycete before contacting said plant, a part thereof or the plant habitat with the at least one bacterial strain, functional homolog thereof, preparation of same, a lysate thereof, an extract thereof, a broth obtained therefrom or a composition comprising same.

According to yet a further aspect, the present invention provides a method for preventing or treating a plant disease caused by a phytopathogenic fungus and/or oomycete, comprising contacting a plant, a part thereof or the plant habitat with at least one bacterial strain selected from the group consisting of LAV58567, LAV104662, LAV104858, LAV104632, LAV104629, LAV1000636, LAV1000506, LAV1005101, LAV1001107, LAV1001081, LAV1000847, LAV1000965, LAV1000933, LAV104661, LAV104665, LAV104794, LAV104618, LAV104692, LAV104891, LAV104630, LAV104664, LAV104922, LAV59524, LAV104961, LAV104960, LAV104533, and functional homologs thereof; a preparation of same, a lysate thereof, an extract thereof, a broth obtained therefrom or a composition comprising same. Each possibility represents a separate embodiment of the present invention.

According to certain exemplary embodiments, the bacterial strain is LAV58567 and/or a functional homolog thereof.

According to certain embodiments, the plant is affected by the disease caused by the phytopathogenic fungus and/or oomycete. According to some embodiments, the method further comprises identifying symptoms of the disease within the plant before contacting said plant, a part thereof or said plant habitat with the at least one bacterial strain, functional homolog thereof, preparation of same, lysate or extract thereof, a broth obtained therefrom or a composition comprising same.

The bacterial strains are as described hereinabove. According to certain embodiments, the methods of the present invention comprise contacting the plant, a part thereof, or the plant habitat with at least one functional homolog of strains LAV58567, LAV104662, LAV104858, LAV104632, LAV104629, LAV1000506, LAV1005101, LAV1001107, LAV1001081, LAV1000847, LAV1000933, LAV104661, LAV104665, LAV104794, LAV104618, LAV104692, LAV104891, LAV104630, LAV104664, LAV104922, LAV59524, LAV104961, LAV104960, and LAV104533, as described hereinabove, preparations of same, lysate thereof, extract thereof, broth obtained therefrom or a composition comprising same.

According to certain embodiments, the plant part is selected from the group consisting of a seed, a root, a shoot, a leaf, a branch, a flower, a fruit and any combination thereof. Each possibility represents a separate embodiment of the present invention.

Any method as is known in the art for contacting bacterial strains with a plant, a part thereof or the plant habitat can be used according to the teachings of the present invention. Typically, the plant or part thereof is contacted with a composition comprising the bacterial strains, functional homologs thereof or preparations of same. According to some embodiments, the composition may be applied to the plant habitat. According to certain embodiments, the plant habitat is a growth medium, which can be a solid or a liquid growth medium. According to certain exemplary embodiments, the solid growth medium is soil.

According to certain embodiments, the composition is formulated in a liquid form. According to these embodiments, the plant or part thereof may be contacted with the composition by a method selected from the group consisting of infiltration, immersion/dipping, incubation, spraying, and any combination thereof. Each possibility represents a separate embodiment of the present invention.

According to certain exemplary embodiments, the plant part is a seed. According to certain embodiments, the at least one bacterial strain according to the teachings of the invention is applied to the seed via seed coating.

According to certain additional or alternative exemplary embodiments, the plant part is a leaf According to some embodiments, the at least one bacterial strain is applied to the leaf by spraying or dusting a bacterial preparation or a composition comprising same according to the teachings of the present invention.

According to certain exemplary embodiments, the plant part is a root and contacting is performed by dipping or immersing.

According to certain embodiments, the bacterial strain is applied at a concentration range of from about 10² CFU/ml to about 10¹⁰ CFU/ml of the bacterial preparation or the agricultural composition. According to some embodiments, the bacterial strain is applied to seeds at a concentration range of from about 10² CFU/seed to about 10¹⁰ CFU/seed. According to certain exemplary embodiments, the bacterial strain is applied at a concentration range of from about 10⁷ CFU/ml to about 10⁸ CFU/ml of the bacterial preparation or the agricultural composition or from about 10⁷ CFU to about 10⁸ CFU per seed.

According to certain aspects, the present invention provides a modified bacterial strain having an improved capability to confer or enhance the resistance of a plant towards pathogenic fungi and/or oomycetes, or to protect the plant from a disease caused by the pathogenic fungi and/or oomycetes as compared to a corresponding unmodified bacterial strain. According to certain currently exemplary embodiments, the modified bacterial strain is a non-genetically modified organism (Non-GMO). According to certain exemplary embodiments, the modified bacterial strain is LAV58567.

According to certain further aspects, the present invention provides a method of obtaining a modified bacterial strain having an improved capability to confer or enhance the resistance of a plant towards pathogenic fungi and/or oomycetes, or to protect the plant from a disease caused by the pathogenic fungi and/or oomycetes, as compared to a parent unmodified bacterial strain, comprising:
(a) culturing a parent bacterial strain having a capability to confer or enhance the resistance of a plant towards pathogenic fungi and/or oomycetes, or to protect the plant from a disease caused by the pathogenic fungi and/or oomycetes under conditions suitable for expanding a population of the bacterial strain and allowing development of at least one bacterial mutant; and
(b) selecting the at least one bacterial mutant resultant of step (a) for an improved capability to confer or enhance the resistance of a plant towards the pathogenic fungi and/or oomycetes, or to protect the plant from a disease caused by said pathogenic fungi and/or oomycetes,
thereby obtaining the modified bacterial strain having the improved capability to confer or enhance the resistance of a plant towards said pathogenic fungi and/or oomycetes, or to protect the plant from a disease caused by said pathogenic fungi and/or oomycetes as compared to the unmodified parent bacterial strain.

According to certain embodiments, the unmodified parent bacterial strain is a strain of the present invention.

According to certain embodiments, the phytopathogenic fungus is of a genus selected from the group consisting of Fusarium, Botrytis, Erysiphe, Aspergillus, and Rhizopus. Each possibility represents a separate embodiment of the present invention.

According to certain embodiments, the phytopathogenic fungus is of a species selected from the group consisting of Fusarium graminearum, Fusarium verticillioides, Fusarium oxysporum, Botrytis cinerea, Erysiphe necator, Aspergillus niger, and Rhizopus stolonifera. According to certain exemplary embodiments, the phytopathogenic fungus is selected from the group consisting of Fusarium graminearum, Fusarium verticillioides, and Botrytis cinerea. Each possibility represents a separate embodiment of the present invention.

According to certain embodiments, the oomycete is of a genus selected from the group consisting of Plasmopara, Pythium, Phytophthora, and Pseudoperonospora. Each possibility represents a separate embodiment of the present invention.

According to certain embodiments, the oomycete is of a species selected from the group consisting of Plasmopara viticola, Pythium aphanidermatum and Pythium irregulare. Each possibility represents a separate embodiment of the present invention.

According to certain exemplary embodiments, the pathogenic fungus is Fusarium graminearum, and the bacterial strain is selected from the group consisting of LAV104794, LAV104632, LAV104664, LAV104630, LAV104891, LAV104922, LAV59524, LAV58567, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic fungus is Fusarium verticillioides, and the bacterial strain is selected from the group consisting of LAV104794, LAV104661, LAV104662, LAV104629, LAV104665, LAV104664, LAV104692, LAV104630, LAV104618, LAV104891, LAV104922, LAV59524, LAV104960, LAV104961 LAV58567, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic fungus is Botrytis cinerea, and the bacterial strain is selected from the group consisting of LAV104794, LAV104661, LAV104662, LAV104632, LAV104665, LAV104858, LAV104692 LAV104630, LAV104618, LAV104891, LAV104922, LAV59524, LAV104960, LAV104961, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic oomycete is Pythium aphanidermatum, and the bacterial strain is selected from the group consisting of LAV104794, LAV104629, LAV104665, LAV104858, LAV104664, LAV104692, LAV104630, LAV104891, LAV104922, LAV59524, LAV104961, LAV58567, LAV104661, LAV104662, LAV104632, LAV104618, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic oomycete is Pythium irregulare, and the bacterial strain is selected from the group consisting of LAV104661, LAV104662, LAV104632, LAV104629, LAV104665, LAV104664, LAV104630, LAV104618, LAV1000506, LAV1000636, LAV1000847, LAV1000933, LAV1001081, LAV1001107, and LAV1005101, functional homologs thereof and any combination thereof. Each possibility represents a separate embodiment of the present invention.

According to certain exemplary embodiments, the pathogenic oomycete is Plasmopara viticola, and the bacterial strain is selected from the group consisting of LAV59524, LAV104960, LAV104961, LAV104533, and LAV58567, functional homologs thereof and any combination thereof. Each possibility represents a separate embodiment of the present invention.

According to certain embodiments, the plant is of a family selected from the group consisting of Actinidiaceae, Amaranthaceae, Anacardiaceae, Apiaceae, Apocynaceae, Araceae, Araliaceae, Arecaceae, Asteraceae, Begoniaceae, Brassicaceae, Cactaceae, Cannabaceae, Cannaceae, Caprifoliaceae, Caryophyllaceae, Casuarinaceae, Chenopodiaceae, Cucurbitaceae, Cyperaceae, Ericaceae, Euphorbiaceae, Fabaceae, Geraniaceae, Gesneriaceae, Ginkgoaceae, Hydrangeaceae, Iridaceae, Juglandaceae, Lamiaceae, Magnoliaceae, Malvaceae, Moraceae, Musaceae, Myrtaceae, Oleaceae, Papaveraceae, Passifloraceae, Pedaliaceae, Piperaceae, Platanaceae, Poaceae, Poaceae, Polygonaceae, Portulacaceae, Proteaceae, Punicaceae, Ranunculaceae, Rosaceae, Rubiaceae, Rutaceae, Salicaceae, Scrophulariaceae, Solanaceae, Sterculiaceae, Taxodiaceae, Theaceae, Tiliaceae, Verbenaceae, Violaceae, Vitaceae, and Zingiberaceae.

According to certain exemplary embodiments, the plant is selected from grape (Vitis vinifera), tomato (Solanum lycopersicum), wheat (Triticum aestivum), and corn (Zea mays).

It is to be understood that any combination of each of the aspects and the embodiments disclosed herein is explicitly encompassed within the disclosure of the present invention.

Other objects, features and advantages of the present invention will become clear from the following description.

### DETAILED DESCRIPTION OF THE INVENTION

The present invention provides isolated microbial strains, particularly bacterial strains, not hitherto described, wherein the bacterial strains are characterized by effective anti-fungal and/or anti-oomycete activity, and therefore are useful as biocontrol agents for protecting plants from diseases caused by the fungi and/or oomycetes. The present invention further provides bacterial strains selected by direct evolution to have improved anti-fungal/anti-oomycete activity. The anti-fungal/anti-oomycete activity encompasses inhibiting at least one of growth, development and phytopathogenic activity of at least one of the fungi/oomycetes.

### Definitions

The terms “comprise”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity, and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “method” refers to manners, means, techniques and procedures for accomplishing a given task including, but not limited to, those manners, means, techniques and procedures either known to, or readily developed from known manners, means, techniques and procedures by practitioners of the agricultural, chemical, pharmacological, biological, biochemical and medical arts.

When reference is made to particular sequence listings, such reference is to be understood to also encompass sequences that substantially correspond to its complementary sequence, as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

As used herein, the terms “sequence identity” or “identity” or grammatical equivalents, in the context of two nucleic acid or polypeptide sequences, includes reference to the residues in the two sequences which are the same when aligned. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity), and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G (Amino acid substitution matrices from protein blocks. Proc. Natl. Acad. Sci. U.S.A. 1992, 89(22): 10915-9).

Identity can be determined using any homology comparison software, including for example, the BlastN software of the National Center of Biotechnology Information (NCBI), such as by using default parameters.

According to some embodiments of the invention, the identity is a global identity, i.e., an identity over the entire amino acid or nucleic acid sequences of the invention and not over portions thereof.

As used herein, the term “query coverage” refers to a percentage that describes how much of the query sequence is covered by the target sequence.

As used herein, the terms “marker”, “genomic marker” and “sub-genomic sequence” are used herein interchangeably and refer to a DNA (deoxyribonucleic acid) sequence present within the genome of a microbial strain.

According to certain exemplary embodiments, identity of a genomic marker sequence is defined as at least 90% query coverage with at least 95% identity, such as further described herein.

The terms “microbial strain(s)” and “bacterial strain(s)” are used herein interchangeably and refer to the bacterial strains of the invention as defined herein.

The terms “functional homolog”, “functionally homologous”, “variant” and grammatical equivalents are used herein interchangeably and refer to a modification (i.e., mutant, at least one mutation) of the bacterial strains of the invention resulting in a microbial strain that is endowed with substantially the same ensemble of biological activities, particularly anti-fungal and/or anti-oomycete activities (+/−10%, 20%, 40%, 50%, or 60% when tested under the same conditions) as that of the strain of the invention, and can be classified to the same species or strain based on known methods of species/strain classifications and as described herein. The modification can be man-made or evolutionary, e.g., during propagation with or without selection.

The terms “isolated” and “biologically pure” with reference to a bacterial strain of the invention relate to bacterium or bacteria of the same species at least partially separated from the natural environment e.g., from the microbial strain habitat or from one or more constituents thereof, cellular or otherwise, with which it may be associated if found in nature. According to certain embodiments, the bacterial strains of the invention are isolated from the plant (the strain being part of the plant microbiome) or from the plant habitat (including the plant rhizoplane and rhizosphere).

As used herein, the term “rhizoplane” refers to the external surface of roots together with closely adhering soil particles and debris.

As used herein, the term “rhizosphere” refers to the region of soil in the vicinity of plant roots, in which the soil chemistry and microbiology is influenced by the plant root growth, respiration, and nutrient exchange.

As used herein, the term “phyllosphere” refers to the total above-ground portions of a plant, which are a habitat for microorganisms. The phyllosphere can be further subdivided into the caulosphere (stems), phylloplane (leaves), anthosphere (flowers), and carposphere (fruit).

The terms “whole cell broth” and “whole cell culture” are used herein interchangeably and refer to a liquid culture containing both cells and a liquid medium.

In particular embodiments, the terms relate to liquid medium comprising at least one bacterial strain of the invention and fermentation products thereof.

Selected strains were isolated and screened according to their ability to inhibit growth of phytopathogenic fungi/oomycetes causing plant diseases with negative effects on crop production. The isolated bacterial strains are described in Tables 1-2 and 4-5, and the strains' inhibiting activities are described in Tables 3 and 6-11 hereinbelow. Also contemplated are functional homologs of the strains as defined and described herein.

According to certain embodiments, the bacterial strain or functional homolog thereof interacting with the host plant is present in the plant habitat, particularly in the rhizosphere (soil around root). According to certain embodiments, the bacterial strain or functional homolog thereof interacting with the host plant is present on or inside a plant tissue, including, but not limited to, the rhizoplane (root surface), root endosphere (inside the root), stem endosphere (inside the stem), leaf endosphere (inside the leaf), phyllosphere (on the shoot, stem and leaf surface), seed surface and seed endosphere (inside the seed). Each possibility represents a separate embodiment of the present invention.

According to certain aspects, the present invention provides an isolated bacterial strain or a functional homolog thereof, wherein the isolated bacterial strain is selected from the group consisting of:
(1) strain LAV58567, the strain being selected from the group consisting of
   a. a strain deposited under Accession Number 43954 at NCIMB;
   b. a strain comprising a 16S-rRNA sequence comprising the nucleic acid sequence set forth in SEQ ID NO:5;
   c. a strain comprising at least one genomic marker comprising the nucleic acid sequence set forth in any one of SEQ ID NOs:36-38;
   and any combination thereof,
(2) strain LAV104661, the strain being selected from the group consisting of
   a. a strain comprising a 16S-rRNA sequence comprising the nucleic acid sequence set forth in SEQ ID NO: 14;
   b. a strain comprising at least one genomic marker comprising the nucleic acid sequence set forth in any one of SEQ ID NOs:67-68;
   and a combination thereof,
(3) strain LAV104629, the strain being selected from the group consisting of
   a. a strain deposited under Accession Number 43958 at NCIMB;
   b. a strain comprising a 16S-rRNA sequence comprising the nucleic acid sequence set forth in SEQ ID NO:1;
   c. a strain comprising at least one genomic marker comprising the nucleic acid sequence set forth in any one of SEQ ID NOs:27-28;
   and any combination thereof;
(4) strain LAV104665, the strain being selected from the group consisting of
   a. a strain comprising a 16S-rRNA sequence comprising the nucleic acid sequence set forth in SEQ ID NO: 15;
   b. a strain comprising at least one genomic marker comprising the nucleic acid sequence set forth in any one of SEQ ID NOs:69-71;
   and a combination thereof,
(5) strain LAV104794, the strain being selected from the group consisting of
   a. a strain comprising a 16S-rRNA sequence comprising the nucleic acid sequence set forth in SEQ ID NO: 16;
   b. a strain comprising at least one genomic marker comprising the nucleic acid sequence set forth in any one of SEQ ID NOs72-74;
   and a combination thereof,
(6) strain LAV104618, the strain being selected from the group consisting of
   a. a strain comprising a 16S-rRNA sequence comprising the nucleic acid sequence set forth in SEQ ID NO: 17;
   b. a strain comprising at least one genomic marker comprising the nucleic acid sequence set forth in any one of SEQ ID NOs:75-77;
   and a combination thereof;
(7) strain LAV104692, the strain being selected from the group consisting of
   a. a strain comprising a 16S-rRNA sequence comprising the nucleic acid sequence set forth in SEQ ID NO: 18;
   b. a strain comprising at least one genomic marker comprising the nucleic acid sequence set forth in any one of SEQ ID NOs:78-80;
   and a combination thereof,
(8) strain LAV104632, the strain being selected from the group consisting of
   a. a strain deposited under Accession Number 43957 at NCIMB;
   b. a strain comprising a 16S-rRNA sequence comprising the nucleic acid sequence set forth in SEQ ID NO:2;
   c. a strain comprising at least one genomic marker comprising the nucleic acid sequence set forth in SEQ ID NO:29;
   and any combination thereof;
(9) strain LAV104891, the strain being selected from the group consisting of
   a. a strain comprising a 16S-rRNA sequence comprising the nucleic acid sequence set forth in SEQ ID NO: 19;
   b. a strain comprising at least one genomic marker comprising the nucleic acid sequence set forth in any one of SEQ ID NOs:81-83;
   and a combination thereof;
(10) strain LAV104630, the strain being selected from the group consisting of
   a. a strain comprising a 16S-rRNA sequence comprising the nucleic acid sequence set forth in SEQ ID NO:20;
   b. a strain comprising at least one genomic marker comprising the nucleic acid sequence set forth in any one of SEQ ID NOs:84-85;
   and a combination thereof;
(11) strain LAV104664, the strain being selected from the group consisting of
   a. a strain comprising a 16S-rRNA sequence comprising the nucleic acid sequence set forth in SEQ ID NO:21;
   b. a strain comprising at least one genomic marker comprising the nucleic acid sequence set forth in any one of SEQ ID NOs:86-88;
   and a combination thereof;
(12) strain LAV104922, the strain being selected from the group consisting of
   a. a strain comprising a 16S-rRNA sequence comprising the nucleic acid sequence set forth in SEQ ID NO:22;
   b. a strain comprising at least one genomic marker comprising the nucleic acid sequence set forth in any one of SEQ ID NOs:89-91;
   and a combination thereof;
(13) strain LAV59524, the strain being selected from the group consisting of
   a. a strain comprising a 16S-rRNA sequence comprising the nucleic acid sequence set forth in SEQ ID NO:23;
   b. a strain comprising at least one genomic marker comprising the nucleic acid sequence set forth in any one of SEQ ID NOs:92-94;
   and a combination thereof;
(14) strain LAV104960, the strain comprising a 16S-rRNA sequence comprising the nucleic acid sequence set forth in SEQ ID NO:24;
(15) strain LAV104858, the strain being selected from the group consisting of
   a. a strain deposited under Accession Number 43956 at NCIMB;
   b. a strain comprising a 16S-rRNA sequence comprising the nucleic acid sequence set forth in SEQ ID NO:3;
   c. a strain comprising at least one genomic marker comprising the nucleic acid sequence set forth in any one of SEQ ID NOs:30-32;
   and any combination thereof;
(16) strain LAV104662, the strain being selected from the group consisting of
   a. a strain deposited under Accession Number 43955 at NCIMB;
   b. a strain comprising a 16S-rRNA sequence comprising the nucleic acid sequence set forth in SEQ ID NO:4;
   c. a strain comprising at least one genomic marker comprising the nucleic acid sequence set forth in any one of SEQ ID NOs:33-35;
   and any combination thereof;
(17) strain LAV104961, the being selected from the group consisting of
   a. a strain comprising a 16S-rRNA sequence comprising the nucleic acid sequence set forth in SEQ ID NO:25;
   b. a strain comprising at least one genomic marker comprising the nucleic acid sequence set forth in any one of SEQ ID NOs:95-97;
   and a combination thereof;
(18) strain LAV104533, the strain comprising a 16S-rRNA sequence comprising the nucleic acid sequence set forth in SEQ ID NO:26;
(19) strain LAV1000636, the strain being selected from the group consisting of
   a. a strain deposited under Accession Number 43964 at NCIMB;
   b. a strain comprising a 16S-rRNA sequence comprising the nucleic acid sequence set forth in SEQ ID NO:6;
   c. a strain comprising at least one genomic marker comprising the nucleic acid sequence set forth in SEQ ID NO:39;
   and any combination thereof;
(20) strain LAV1000506, the strain being selected from the group consisting of
   a. a strain deposited under Accession Number 43965 at NCIMB;
   b. a strain comprising a 16S-rRNA sequence comprising the nucleic acid sequence set forth in SEQ ID NO:7;
   c. a strain comprising at least one genomic marker comprising the nucleic acid sequence set forth in any one of SEQ ID NOs:40-42;
   and any combination thereof;
(21) strain LAV1005101, the strain being selected from the group consisting of:
   a. a strain deposited under Accession Number 43959 at NCIMB;
   b. a strain comprising a 16S-rRNA sequence comprising the nucleic acid sequence set forth in SEQ ID NO: 13;
   c. a strain comprising at least one genomic marker comprising the nucleic acid sequence set forth in any one of SEQ ID NOs:62-66;
   and any combination thereof;
(22) strain LAV1001107, the strain being selected from the group consisting of:
   a. a strain deposited under Accession Number 43960 at NCIMB;
   b. a strain comprising a 16S-rRNA sequence comprising the nucleic acid sequence set forth in SEQ ID NO: 12;
   c. a strain comprising at least one genomic marker comprising the nucleic acid sequence set forth in any one of SEQ ID NOs:57-61;
   and any combination thereof;
(23) strain LAV1001081, the strain being selected from the group consisting of:
   a. a strain deposited under Accession Number 43961 at NCIMB;
   b. a strain comprising a 16S-rRNA sequence comprising the nucleic acid sequence set forth in SEQ ID NO: 11;
   c. a strain comprising at least one genomic marker comprising the nucleic acid sequence set forth in SEQ ID NO:56;
   and any combination thereof;
(24) strain LAV1000847, the strain being selected from the group consisting of:
   a. a strain deposited under Accession Number 43962 at NCIMB;
   b. a strain comprising a 16S-rRNA sequence comprising the nucleic acid sequence set forth in SEQ ID NO:9;
   c. a strain comprising at least one genomic marker comprising the nucleic acid sequence set forth in any one of SEQ ID NOs:48-50;
   and any combination thereof;
(25) strain LAV1000965, the strain being selected from the group consisting of:
   a. a strain deposited under Accession Number 43963 at NCIMB;
   b. a strain comprising a 16S-rRNA sequence comprising the nucleic acid sequence set forth in SEQ ID NO:8;
   c. a strain comprising at least one genomic marker comprising the nucleic acid sequence set forth in any one of SEQ ID NOs:43-47;
   and any combination thereof;
(26) strain LAV1000933, the strain being selected from the group consisting of:
   a. a strain comprising a 16S-rRNA sequence comprising the nucleic acid sequence set forth in SEQ ID NO: 10; and
   b. a strain comprising at least one genomic marker comprising the nucleic acid sequence set forth in any one of SEQ ID NOs:51-55;
   and a combination thereof.

Each possibility represents a separate embodiment of the present invention.

The criteria for identifying a functional homolog of a strain of the invention include functional and/or genetic criteria as are known to the skilled Artisan.

Bacterial strains of Accession Numbers 43954, 43955, 43956, 43957, 43958, 43959 43960, 43961, 43962, 43963, 43964 were deposited at NCIMB Ltd., Ferguson Building, Craibstone Estate, Bucksburn, Aberdeen, AB21 9YA Scotland, United Kingdon, on Mar. 23, 2022; Bacterial strains of Accession numbers 43965 was deposited at NCIMB Ltd., Ferguson Building, Craibstone Estate, Bucksburn, Aberdeen, AB21 9YA Scotland, United Kingdon, on Mar. 30, 2022.

According to certain embodiments, the strain of the invention and the functional homolog belong to the same operational taxonomic units (OTU).

An “OTU″” (or plural, “OTUs”) refers to a terminal leaf in a phylogenetic tree and is defined by a nucleic acid sequence, e.g., the entire genome or a specific genetic sequence, and all sequences that share sequence identity to this nucleic acid sequence at the level of species. According to certain exemplary embodiments, the specific genetic sequence may be the 16S-rRNA sequence or a portion of the 16S-rRNA (also referred to herein as “16S”) sequence, or other functionally conserved sequences as listed below. According to additional exemplary embodiments, the entire genomes of two entities are sequenced and compared. In another embodiment, selected regions such as multilocus sequence tags (MLST, MLSA), specific genes, or sets of genes may be genetically compared. For 16S-rRNA sequences, OTUs that share at least 97% average nucleotide identity across the entire 16S or some variable region of the 16S are considered the same OTU (see e.g., Claesson M J, et al. 2010. Nucleic Acids Res 38:e200; Konstantinidis K T, et al. 2006. Philos Trans R Soc Lond B Biol Sci 361:1929-1940). In embodiments involving the complete genome, MLSTs, specific genes, or sets of genes, OTUs that share at least 95% average nucleotide identity are considered the same OTU (see e.g., Achtman M, and Wagner M. 2008. Nat. Rev. Microbiol. 6:431-440; Konstantinidis et al. 2006, ibid). OTUs are frequently defined by comparing sequences between organisms. Such characterization employs, e.g., whole genome sequencing (WGS) data.

According to certain embodiments, the functional homolog comprises a functionally conserved gene or a fragment thereof. According to certain embodiments, the functionally conserved gene is a house-keeping gene selected from the group consisting of, but not limited to, 16S-rRNA, recA, glnII, atpD, gap, glnA, gltA, gyrB, pnp, rpoB, thrC and dnaK, that is at least about 97%, at least about 98%, at least about 99%, at least about 99.1%, at least about 99.5%, at least about 99.6%, at least about 99.7%, at least about 99.8%, at least about 99.9% or more, or fully identical to that of a strain of the invention as described herein. Each possibility represents a separate embodiment of the present invention.

As mentioned, and according to a specific additional or an alternative embodiment, a functional homolog can also be determined on the basis of a multilocus sequence analysis (MHLSA) determination of various functionally conserved genes or fragments thereof, e.g., at least one, 2, 3, 4, 5, 6, 7, 8, 9, 10, 11, 12, 13, 14, 15 or more functionally conserved genes or fragments thereof, such as of e.g., 16S, recA, glnII, atpD, gap, glnA, gltA, gyrB, pnp, rpoB, thrC and dnaK.

According to certain exemplary embodiments, the house-keeping gene is 16S ribosomal RNA (16S-rRNA).

According to certain exemplary embodiments, the identity of the 16S sequence is defined as at least 100% query coverage with at least 97% identity, at least 97.5% identity, at least 98% identity, at least 98.5% identity, at least 99% identity, at least 99.5% identity or more.

According to certain embodiments, the functional homolog bacterial strain comprises a 16S-rRNA sequence at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical to the 16S-rRNA sequence of a strain of the invention, said 16S-rRNA sequence of the strain of the invention comprising a nucleic acid sequence selected from the group consisting of SEQ ID NOs:1-4, 6-12, 15-18, 20, 23, 25-27. Each possibility represents a separate embodiment of the present invention.

According to certain embodiments, the functional homolog bacterial strain comprises a 16S-rRNA sequence at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homology, or identity to the 16S-rRNA sequence of strain LAV104618, having SEQ ID NO:17.

According to certain embodiments, the functional homolog bacterial strain comprises a 16S-rRNA sequence at least about at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homology, or identity to the 16S-rRNA sequence of strain LAV1000965, having SEQ ID NO:8.

According to certain embodiments, the functional homolog bacterial strain comprises a 16S-rRNA sequence at least about at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homology, or identity to the 16S-rRNA sequence of strain LAV104858, having SEQ ID NO:3.

According to certain embodiments, the functional homolog bacterial strain comprises a 16S-rRNA sequence at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homology, or identity to the 16S-rRNA sequence of strain LAV1000636, having SEQ ID NO:6.

According to certain embodiments, the functional homolog bacterial strain comprises a 16S-rRNA sequence at least about 99.5%, at least about 99.6%, at least about 99.7%, at least about 99.8%, at least about 99.9% or more homology, or identity to the 16S-rRNA sequence of strain LAV1000847, having SEQ ID NO:9.

According to certain exemplary embodiments, the bacterial strain comprises more than one 16S-rRNA.

According to certain additional or alternative embodiments, the genomic nucleic acid sequences of the bacterial strain and the functional homolog thereof comprise at least one strain-specific genomic marker.

According to certain embodiments, the microbial strain of the present invention or the functional homolog thereof comprises at least two genomic markers, at least three genomic markers, at least four genomic markers, or at least five genomic markers.

According to certain exemplary embodiments, the functional homolog of a bacterial strain of the invention comprises at least one genomic marker selected from the group consisting of a marker having a nucleic acid sequence at least about 95%, at least about 95.5%, at least about 96%, at least about 96.5%, at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9%, at least about 99%, at least about 99.1%, at least about 99.2%, at least about 99.3%, at least about 99.4%, at least about 99.5%, at least about 99.6%, at least about 99.7%, at least about 99.8%, at least about 99.9% or more homologous, or identical, to any one of SEQ ID NOs:27-97.

According to certain embodiments, strain LAV104794 and strain LAV1000636 are functional homologs, wherein the 16S-rRNA of LAV104794 having the nucleic acid sequence set forth in SEQ ID NO:16 and the 16S-rRNA of LAV1000636 having the nucleic acid sequence set forth in SEQ ID NO:6 share over 97% sequence identity.

According to certain embodiments, strain LAV104891 and strain LAV104922 are functional homologs, wherein the 16S-rRNA of LAV104891 having the nucleic acid sequence set forth in SEQ ID NO:19 and the 16S-rRNA of LAV104922 having the nucleic acid sequence set forth in SEQ ID NO:22 share over 97% sequence identity.

According to certain embodiments, the microbial strain of the present invention and the functional homolog thereof share at least two genomic markers, at least three genomic markers, at least four genomic markers, or at least five genomic markers.

According to additional or alternative embodiments, the strain of the invention and the functional homolog are characterized by substantially the same (+/−about 10%, 20%, 40%, 50%, 60% when tested under the same conditions) biochemical profiling (e.g., biochemical fingerprinting) using for example, the GEN III redox chemistry MicroPlate (BIOLOG Inc. 21124 Cabot Blvd. Hayward CA, USA), which can analyze both Gram-negative and Gram-positive bacteria, for their ability to metabolize all major classes of biochemicals, in addition to determining other important physiological properties such as pH, salt, and lactic acid tolerance. Further details of biochemical profiling can be obtained in “Modern Phenotypic Microbial Identification”, Bochner B. R., Encyclopedia of Rapid Microbiological Methods, 2006, v. 2, Ch. 3, pp. 55-73.

Genomic data can be obtained by methods which are well known in the art, e.g., DNA sequencing, bioinformatics, electrophoresis, enzyme-based mismatch detection assay and a hybridization assay; including, e.g., PCR, RT-PCR, RNase protection, in-situ hybridization, primer extension, Southern blot, Northern Blot, dot blot analysis and the like.

According to certain exemplary embodiments, the functional homolog and the strain of the invention belong to the same genus. According to further certain exemplary embodiments, the functional homolog and the strain of the invention belong to the same species or the same sub-species.

According to certain embodiments, the bacterial strains of the present invention are of a genus selected from the group consisting of Actinokineospora, Acinetobacter, Bacillus, Ensifer, Enterobacter, Erwinia, Flavobacterium, Gordonia, Lysobacter, Paenibacillus, Pantoea, Pseudomonas, Rahnella, Sphingobium and Streptomyces.

According to certain embodiments, strain LAV104661 is of the genus Erwinia. According to certain embodiments, strains LAV104629 and LAV104692 each is of the genus Pantoea. According to certain embodiments, strains LAV104665, LAV104794, LAV104664, LAV104858, LAV1000636, LAV1001107, LAV1000847, and LAV1000965 each is of the genus Pseudomonas. According to certain embodiments, strain LAV104618 is of the genus Ensifer. According to certain embodiments, strains LAV104632 and LAV59524 each is of the genus Bacillus. According to certain embodiments, strains LAV104891 and LAV104922 each is of the genus Acinetobacter. According to certain embodiments, strain LAV105630 is of the genus Enterobacter. According to certain embodiments, strains LAV104960 and LAV104961 each is of the genus Streptomyces. According to certain embodiments, strain LAV104662 is of the genus Sphingobium. According to certain embodiments, strain LAV104533 is of the genus Actinokineospora. According to certain embodiments, strain LAV58567 is of the genus Lysobacter. According to certain embodiments, strain LAV1000506 is of the genus Gordonia. According to certain embodiments, strain LAV1005101 is of the genus Pseudoxanthomonas. According to certain embodiments, strain LAV1001081 is of the genus Rahnella. According to certain embodiments, strain LAV1000933 is of the genus Paenibacillus.

As used herein, the term “preparation” refers to an isolate of bacteria in which the prevalence (i.e., concentration and/or ratio) of the bacterial strain or functional homolog is enriched over that (exceeds that) found in nature. In nature, the bacterial strain is typically part of the plant microbiome, consisting of more than thousands of microbial species, whether in the phyllosphere, including endophytes, rhizoplane, rhizosphere or any other plant compartment. According to some embodiments of the invention, the preparation comprises less than 50, 20, 10, 9, 8, 7, 6, 5, or 4 microbial species, e.g., bacteria and fungi.

According to certain exemplary embodiments, the microbial preparations comprise 1, 2, 3, 4, 5, 6, 7, 8, 9 or 10 microbial species. Each possibility represents a separate embodiment of the present invention.

According to further exemplary embodiments, the microbial preparation comprises a single microbial species, i.e., bacteria according to the teachings of the present invention.

According to certain exemplary embodiments, the preparation comprises a single strain of the present invention, wherein said preparation is devoid of other microbial species. According to certain further exemplary embodiments, the preparation comprises a plurality of strains of the present invention, wherein said preparation is devoid of other microbial species.

According to certain embodiments, the preparation comprises the bacterial strain of the invention at a level of purity of at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95% or more, say 100% pure. As used herein, the term “pure” or “purity” refers to the percentage of the bacterial strain of the invention out of the total number of microorganisms in the preparation.

According to certain exemplary embodiments, the preparation comprises the bacterial strain of the invention at a level of purity of at least about 99%, at least about 99.1%, at least about 99.2%, at least about 99.3%, at least about 99.4%, at least about 99.5%, at least about 99.6%, at least about 99.7%, at least about 99.8%, at least about 99.9%, at least about 99.95%, at least about 99.99%, at least about 99.999% or more, say 100% pure.

As used herein, the term “enriched” refers to 2-10×10⁶-fold enrichment over that found in nature in an isolate of microbiota obtained from a plant phyllosphere and/or rhizosphere comprising a strain of the invention or a functional homolog of same.

A “spore” or “spores” refers to microbial structures that are generally viable, more resistant to environmental influences such as heat and bactericidal agents than other forms of the same microbial species, and typically capable of germination and out-growth. Bacteria that are “capable of forming spores” are those bacteria comprising the genes and other necessary abilities to produce spores under suitable environmental conditions.

As used herein, the term “culture” refers to a fluid, pellet, scraping, dried sample, lyophilizate or a support, container, or medium such as a plate, paper, filter, matrix, straw, pipette or pipette tip, fiber, needle, gel, swab, tube, vial, particle, etc. that contains the strain or the functional homolog thereof in an amount that exceeds that found in nature, as described hereinabove. In the present invention, an “isolated culture” of a microbial strain is a culture fluid or a scraping, pellet, dried preparation, lyophilizate, or a support, container, or medium that contains the strain or the functional homolog thereof, in the absence of other microorganisms.

Cultures of the strains or of functional homologs thereof may be prepared for use according to the teachings of the invention using standard fermentation techniques known in the art. Growth is commonly performed in a bioreactor.

A bioreactor refers to any device or system that supports a biologically active environment. As described herein, a bioreactor is a vessel in which microorganisms, including the microorganism of the invention, can be grown. A bioreactor may be any appropriate shape or size for growing the microorganisms. A bioreactor may range in size and scale from 10 mL (e.g., small scale) to liters to cubic meters (e.g., large scale) and may be made of stainless steel, disposable material (e.g., nylon, plastic bags) or any other appropriate material as known and used in the art. The bioreactor may be a batch type bioreactor, a fed batch type or a continuous-type bioreactor (e.g., a continuous stirred reactor). For example, a bioreactor may be a chemostat as known and used in the art of microbiology for growing and harvesting microorganisms. A bioreactor may be obtained from any commercial supplier (See also Bioreactor System Design, Asenjo and Merchuk, CRC Press, 1995).

For small scale operations, a batch bioreactor may be used, for example, to test and develop new processes, and for processes that cannot be converted to continuous operations.

Microorganisms grown in a bioreactor may be suspended or immobilized. Growth in the bioreactor is generally under aerobic conditions at suitable temperatures and pH for growth. For the organisms of the invention, cell growth can be achieved at temperatures between 5-37° C., with an exemplary temperature range selected from 15 to 30° C., 15 to 28° C., 20 to 30° C., or 15 to 25° C. The pH of the nutrient medium can vary between 4.0 and 9.0. For example, the operating range can be usually slightly acidic to neutral at pH 4.0 to 7.0, or 4.5 to 6.5, or pH 5.0 to 6.0. Typically, maximal cell yield is obtained in 20-72 hours after inoculation.

Optimal conditions for the cultivation of the microorganisms of this invention will, of course, depend upon the particular strain and strain species. However, by virtue of the conditions applied in the selection process and general requirements of most microorganisms, a person of ordinary skill in the art would be able to determine essential nutrients and conditions. The microorganisms would typically be grown in aerobic liquid cultures on media which contain sources of carbon, nitrogen, and inorganic salts that can be assimilated by the microorganism and supportive of efficient cell growth. Exemplary carbon sources are hexoses such as glucose, but other sources that are readily assimilated, such as amino acids, may form a substitute. Many inorganic and proteinaceous materials may be used as nitrogen sources in the growth process. Exemplary nitrogen sources are amino acids and urea, but others include gaseous ammonia, inorganic salts of nitrate and ammonium, vitamins, purines, pyrimidines, yeast extract, beef extract, proteose peptone, soybean meal, hydrolysates of casein, distiller's solubles, and the like. Among the inorganic minerals that can be incorporated into the nutrient medium are the customary salts capable of yielding calcium, zinc, iron, manganese, magnesium, copper, cobalt, potassium, sodium, molybdate, phosphate, sulfate, chloride, borate, and like ions.

The culture can be a pure culture, whereby a single microbial strain is grown, or a mixed culture. A mixed culture can be prepared pending the compliance of the microbial strains to co-exist and proliferate under the same culturing conditions. When needed, an antibiotic or other growth-restricting conditions, e.g., temperature, essential nutrients and the like can be employed during culturing to restrict the growth of other microorganisms (contaminants) not desired in the culture/co-culture.

According to alternative or additional embodiments, a desired strain combination is produced following culturing. Typically, a strain combination is made after culturing when the microbial strains do not share the same or optimal culturing conditions.

When a combination of strains is to be used, the ratio of each type of microorganism in the final product will depend on the target fungi/oomycetes to be eradicated.

It is to be explicitly understood that the present invention encompasses complete cultures comprising a growth medium and at least one bacterial strains or functional homologs of the invention, as well as growth medium obtained after removal of the bacterial strains. According to certain embodiments, the growth medium obtained after removal of the at least one bacterial strain encompasses fermentation products of said at least one bacterial cell.

According to certain additional aspects, the present invention provides a lysate of at least one bacterial strain of the invention or the functional homologs thereof. According to the certain embodiments, the lysate is of a single strain. According to some embodiments, the lysate comprises a whole cell lysate of the bacterial cells. According to some embodiments, the lysate comprises a soluble fraction of the bacterial cells. According to some embodiments, the lysate comprises inclusion bodies of a bacterial preparation.

According to certain further aspects, the present invention provides a cell extract of at least one bacterial strain of the invention or the functional homologs thereof.

According to certain yet additional aspects, the present invention provides a whole cell broth collected from fermentation of at least one bacterial strain of the invention.

According to certain embodiments, the lysate, extract or broth is obtained from a plurality of the bacterial cells. According to certain embodiments, the lysate, extract or broth is of bacterial cells of the same bacterial species and/or strain. According to certain embodiments, the lysate, extract, or broth is of bacterial cells of different species and/or strains.

The bacterial strain and functional homologs thereof are as described hereinabove.

According to an additional aspect, the present invention provides a composition comprising the bacterial preparation, culture, culture medium, lysate or extract as described herein, further comprising agriculturally acceptable carriers and/or diluents.

According to certain embodiments, the composition further comprises an agriculturally effective amount of an active agent selected from the group consisting of a fertilizer, an acaricide, a bactericide, a fungicide, an insecticide, a microbicide, a nematicide, a pesticide, a plant growth regulator, a rodenticide, and a nutrient.

Also provided is a formulation comprising the bacterial preparation or the composition comprising same as described herein.

Any carrier suitable for agricultural use can form part of the compositions and/or formulations of the present invention. The carrier may be any one or more of a number of carriers that confer a variety of properties, including increased stability, wettability, dispersibility, etc. Wetting agents such as natural or synthetic surfactants, which can be nonionic or ionic surfactants, or a combination thereof, can be included in a composition of the invention. Water-in-oil emulsions can also be used to formulate a composition that includes at least one isolated microorganism of the present invention (see, for example, U.S. Pat. No. 7,485,451). Suitable formulations that may be prepared include wettable powders, granules, gels, agar strips or pellets, and the like, microencapsulated particles, and the like, liquids such as aqueous flowables, aqueous suspensions, water-in-oil emulsions, etc. The formulation may include grain or legume products (e.g., ground grain or beans, broth or flour derived from grain or beans), starch, sugar, or oil. The carrier may be an agricultural carrier. In certain preferred embodiments, the carrier is a seed, and the composition may be applied or coated onto the seed or allowed to saturate the seed.

According to some embodiments, the agricultural carrier may be soil or plant growth medium. Other agricultural carriers that may be used include water, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as, but not limited to, pesta (flour and kaolin clay), agar or flour-based pellets in loam, sand, clay, etc. Formulations may include food sources for the cultured organisms, such as barley, rice, or other biological materials such as seed, plant parts, sugar cane bagasse, hulls or stalks from grain processing, ground plant material (“yard waste”) or wood from building site refuse, sawdust or small fibers from recycling of paper, fabric, or wood. Other suitable formulations will be known to those skilled in the art.

In the liquid form, e.g., solutions or suspensions, the microbial strain may be mixed or suspended in water or in aqueous solutions. Suitable liquid diluents or carriers include water, aqueous solutions, petroleum distillates, or other liquid carriers.

Solid compositions can be prepared by dispersing the microbial strain in and on an appropriately divided solid carrier, such as peat, wheat, bran, vermiculite, clay, talc, bentonite, diatomaceous earth, fuller's earth, pasteurized soil, and the like. When such formulations are used as wettable powders, biologically compatible dispersing agents such as non-ionic, anionic, amphoteric, or cationic dispersing and emulsifying agents can be used.

According to certain embodiments, the bacterial strains of the present invention are applied within a “dehydrated microfermentor” as described in International (PCT) Application No. PCT/IL2019/050838. As used herein, this term refers to a dehydrated composition comprising a particle encapsulating one or more microorganisms, wherein the particle is composed of an inner core comprising the one or more microorganisms surrounded by an outer shell layer, wherein said outer shell layer is selectively permeable to a rehydrating fluid, and wherein upon fluid absorption, said outer shell layer degrades at a predetermined rate, thereby releasing a plurality of microorganisms to the surrounding environment in a controlled manner. According to certain embodiments, the encapsulated microorganisms, particularly the bacterial strains and functional homologs of the invention, are present in the dehydrated composition at an initial concentration of less than about 1×10³ CFU, and, following fluid absorption, the concentration of the encapsulated microorganisms is increased by at least 10-fold before the microorganisms are released to the surrounding environment. According to certain exemplary embodiments, the concentration of the released microorganisms is at least 10⁵ CFU. In certain embodiments, the dehydrated composition comprises a plurality of particles.

Any fertilizer as is known in the art can be added to the compositions/formulations of the present invention, as long as the fertilizer does not interfere with the bacterial growth and activity. According to certain embodiments, the fertilizer is selected from the group consisting of chemical or biological fertilizer. The amount of the at least one chemical or biological fertilizer employed can vary depending on the final formulation as well as the size of the plant and/or seed to be treated.

A variety of chemical pesticides is apparent to one of skill in the art and may be used. Exemplary chemical pesticides include acylalanines, butyrolactones, oxazolidinones, hydroxy-(2-amino-) pyrimidines, isothiazolones, isoxazoles, carboxylic acids, benzimidazoles, thiophanates, N-phenyl carbamates, toluamides, ethylamino-thiazole-carboxamide, phenylureas, pyridinylmethyl-benzamides, aminocyanoacrylates, benzophenone, benzoylpyridine, pyrazole-5-carboxamides, pyrimidinamines, quinazoline, N-methoxy-(phenyl-ethyl)-pyrazole-carboxamides, furan-carboxamides, oxathiin-carboxamides, phenyl-benzamides, phenyl-oxo-ethyl thiophene amide, pyrazole-4-carboxamides, N-cyclopropyl-N-benzyl-pyrazole-carboxamides, pyridine-carboxamides, pyridinyl-ethyl-benzamides, thiazole-carboxamides, pyrazine-carboxamides, benzyl-carbamates, dihydro-dioxazines, imidazolinones, methoxy-acetamide, methoxy-acrylates, methoxy-carbamates, oxazolidine-diones, oximino-acetamides, oximino-acetates, tetrazolinones, cyano-imidazole, sulfamoyl-triazole, picolinamides, dinitrophenyl crotonates, 2,6-dinitro-anilines, tri-phenyl tin compounds, thiophene-carboxamides, triazolo-pyrimidylamine, anilino-pyrimidines, enopyranuronic acid, hexopyranosyls, glucopyranosyls, tetracycline, aryloxyquinoline, quinazolinone, phenylpyrroles, dicarboximides, dithiolanes, phosphoro-thiolates, aromatic hydrocarbons, 1,2,4-thiadiazoles, carbamates, piperidinyl-thiazole-isoxazolines, imidazoles, piperazines, pyridines, pyrimidines, triazoles, triazolinthiones, morpholines, piperidines, spiroketal-amines, amino-pyrazolinone, hydroxyanilides, allylamines, thiocarbamates, peptidyl pyrimidine nucleoside, cinnamic acid amides, mandelic acid amides, valinamide carbamates, isobenzo-furanone, pyrrolo-quinolinone, triazolobenzo-thiazole, carboxamide, cyclopropane-carboxamide, propionamide, trifluoroethyl-carbamate, benzothiadiazole, thiadiazole-carboxamide, ethyl phosphonates, cyanoacetamide-oxime, phthalamic acids, benzotriazines, benzene-sulfonamides, pyridazinones, phenyl-acetamide, guanidines, cyano-methylene-thiazolidines, pyrimidinone-hydrazones, 4-quinolyl-acetates, tetrazolyloximes, dithiocarbamates, phthalimides, chloronitriles, sulfamides, bis-guanidines, triazines, quinones, quinoxalines, maleimide and phthalonitriles, as well as copper fungicides (acypetacs-copper, Bordeaux mixture, Burgundy mixture, Cheshunt mixture, copper acetate, basic copper carbonate, copper hydroxide, copper naphthenate, copper oleate, copper oxychloride, copper silicate, copper sulfate, basic copper sulfate, copper zinc chromate, cufraneb, cuprobam, cuprous oxide, mancopper, oxine-copper, saisentong, thiodiazole-copper) and other fungicides, including dimethomorph, fosetyl-Al, tetraconazole, azoxystrobin, propineb, pyraclostrobin, potassium phosphite, mefenoxam, and folpet.

The formulation as used herein can also refer to a customary formulation in an effective amount to be applied either to the soil (i.e., in-furrow), to a portion of the plant (i.e., drench) or on the seed before planting (i.e., seed coating or dressing). Customary formulations include solutions, emulsifiable concentrates, wettable powders, suspension concentrates, soluble powders, granules, suspension-emulsion concentrates, natural and synthetic materials impregnated with active compounds, and very fine controlled release capsules in polymeric substances. In certain embodiments of the present invention, the microbial strains are formulated in powders that are available in either a ready-to-use formulation or are otherwise mixed together at the time of use. In either embodiment, the powder may be admixed with the soil prior to or at the time of planting.

Depending on the final formulation, one or more suitable additives can also be introduced to the compositions of the present invention. Adhesives such as carboxymethylcellulose and natural and synthetic polymers in the form of powders, granules or latexes, such as gum arabic, chitin, polyvinyl alcohol and polyvinyl acetate, as well as natural phospholipids, such as cephalins and lecithins, and synthetic phospholipids, can be added to the compositions/formulation of the present invention.

According to certain embodiments, the bacterial strains are formulated in a single, stable solution, or emulsion, or suspension. For solutions, the chemical compounds are typically dissolved in solvents before the microbial strain is added. Suitable liquid solvents include petroleum-based aromatics, such as xylene, toluene or alkylnaphthalenes; aliphatic hydrocarbons, such as cyclohexane or paraffins, for example petroleum fractions; mineral and vegetable oils; alcohols, such as butanol or glycol as well as their ethers and esters; ketones, such as methyl ethyl ketone, methyl isobutyl ketone or cyclohexanone; and strongly polar solvents, such as dimethylformamide and dimethyl sulphoxide. For emulsion or suspension, the liquid medium is water. In some embodiments, the chemical agent and the microbial strain are suspended in separate liquids and mixed at the time of application. In a preferred embodiment of suspension, the chemical agent and the microbial strain are combined in a ready-to-use formulation that exhibits a reasonably long shelf-life. In use, the liquid can be sprayed or can be applied to the plant foliage as an atomized spray or in-furrow at the time of planting the crop. The liquid composition can be introduced in an effective amount on the seed (i.e., seed coating or dressing) or to the soil (i.e., in-furrow) before germination of the seed or directly to the soil in contact with the roots by utilizing a variety of techniques known in the art including, but not limited to, drip irrigation, sprinklers, soil injection or soil drenching. Optionally, stabilizers and buffers can be added, including alkaline and alkaline earth metal salts and organic acids, such as citric acid and ascorbic acid, inorganic acids, such as hydrochloric acid or sulfuric acid. Biocides can also be added and can include formaldehydes or formaldehyde-releasing agents and derivatives of benzoic acid, such as p-hydroxybenzoic acid.

The amount of the bacterial strain or functional homolog within the composition/formulation is sufficient to interact, colonize and/or localize in a cultivated plant treated with same.

According to certain embodiments, the bacterial strain(s) is about 2% w/w to about 80% w/w of the entire formulation/composition. According to other embodiments, the bacterial strains(s) employed in the compositions is about 5% w/w to about 65% w/w or about 10% w/w to about 60% w/w by weight of the entire formulation/composition.

According to certain embodiments, the preparation/composition provided herein is formulated to provide stability for the bacterial strain or functional homolog. Optionally, a shelf-stable formulation is in a dry form, e.g., a powder formulation, or a lyophilized formulation. According to certain embodiments, the formulation is substantially stable at temperatures between about 4° C. and about 37° C. for at least about 5, 10, 15, 20, 25, 30 or more days. According to certain exemplary embodiments, the microbial strain or functional homolog may be shelf-stable, wherein at least 0.01% of the CFU or spores are viable after storage in desiccated form (i.e., moisture content of 30% or less) for 1, 2, 3, 4, 5, 6, 7, 8, 9, 10 or more than 10 weeks at 4° C. or at room temperature.

According to certain embodiments, the carrier is a plant seed. According to these embodiments, the present invention provides an agricultural composition comprising at least one plant seed and at least one bacterial strain selected from the group consisting of LAV104661, LAV104629, LAV104665, LAV104794, LAV104618, LAV104692, LAV104632, LAV104891, LAV104630, LAV104664, LAV104922, LAV59524, LAV104960, LAV104858, LAV104662, LAV104961, LAV104533, LAV58567, LAV1000636, LAV1000506, LAV1005101, LAV1001107, LAV1001081, LAV1000847, LAV1000965, LAV1000933, LAV100930, and functional homologs thereof; a preparation of same, a lysate, a broth or an extract thereof. Each possibility represents a separate embodiment of the present invention.

According to certain currently exemplary embodiments, the agricultural composition is in a form of seed coating. According to these embodiments, the seed coating formulation further comprises at least one agent selected from the group consisting of a binding agent and a wetting agent. According to certain exemplary embodiments, the binding agent is carboxymethyl cellulose (CMC).

According to yet additional certain aspects, the present invention provides a seed coated with an agricultural composition comprising at least one bacterial strain, a lysate, an extract or a broth thereof as described herein.

According to certain aspects, the present invention provides a container adapted for a watering system of a plant field, comprising an agricultural composition comprising at least one bacterial strain, a lysate, an extract or a broth thereof as described herein.

According to certain aspects, the present invention provides a kit comprising (i) an agricultural composition comprising at least one bacterial strain, a lysate, an extract or a broth thereof as described herein, optionally (ii) a delivery system for applying the agricultural composition to a plant or a part thereof or to the plant growth medium, and (iii) instructions for using the agricultural composition.

According to certain embodiments, the instructions for using the agricultural composition comprise instructions for the amounts and frequency of applying the agricultural composition so as to confer or enhance the resistance of the plant towards pathogenic fungi and/or oomycetes.

The bacterial strains of the present invention and their functional homologs are effective in conferring resistance towards phytopathogenic fungi and/or oomycetes to plants contacted with the strain preparations or compositions/formulations comprising same.

As used herein, the terms “conferred resistance to a pathogenic fungus and/or oomycete” or “enhanced resistance to a pathogenic fungus and/or oomycete” refer to a phenotype in which a plant contacted with strains of the present invention has less severe symptoms, and optionally at least one of greater health, growth, propagation, fertility, vigor, strength (e.g., stem strength and resistance), and yield, associated with infection of the pathogenic fungus or oomycete during or after the fungal or oomycete infection than a plant that was not contacted with the strain. A treated plant with enhanced resistance to a fungal or oomycete pathogen can be infected by the pathogen and exhibit one or more symptoms of infection by the pathogen, and yet exhibit a reduction in an effect of the infection or symptom thereof. For instance, a treated plant can be infected by the pathogen, and exhibit one or more symptoms selected from the group consisting of leaf wilt, leaf or vascular discoloration (e.g., yellowing), spike bleaching etc., and yet not exhibit a reduction in yield loss in comparison to a plant that has not been contacted with a strain or strains of the invention.

When a strain of the invention is tested for its capability to confer resistance on a plant or to enhance resistance of a plant, the symptoms associated with the fungal or oomycete infection are compared between a plant or a part thereof treated by contacting the plant or part thereof with strain(s) of the invention and a control plant or part thereof that was not contacted with the strain(s). The control plant is typically, but not necessarily, of the same species as the treated plant. According to some embodiments, the control plant is of the same species and has the same genetic background as the treated plant. The enhancement can be manifested as an increase of 0.1%, 0.2%, 0.3%, 0.5%, 0.75%, 1%, 1.5%, 2%, 3%, 4%, 5%, 6%, 7%, 8%, 9%, 1%, %12%, 15%, 17%, 20%, 25%, 30%, 35%, 40%, 50%, 60%, 70%, 80%, 90%, or 100% in health, growth, multiplication, fertility, vigor, strength, or yield, as compared to a control plant. The enhancement can be a decrease of 0.1%, 0.2%, 0.3%, 0.5%, 0.75%, 1%, 1.5%, 2%, 3%, 4%, 5%, 6%, 7%, 8%, 9%, 10%, 12%, 15%, 17%, 20%, 25%, 30%, 35%, 40%, 50%, 60%, 70%, 80%, 90%, or 100% in the symptoms associated with the pathogenic fungus and/or oomycete as compared to the control plant. According to certain exemplary embodiments, the treated plant and the control plant are grown under the same conditions. According to further exemplary embodiments, the treated plant and the control plant are of the same species.

Symptoms associated with diseases caused by fungal or oomycete pathogens are known to a person skilled in the art. Typically, a score scale is set for a certain disease based on symptom description and/or severity. Optionally, a specific score scale is set for a combination of a certain disease and a plant species. The use of score scales simplifies the comparison of disease symptoms in plants subjected to various treatments.

According to a further aspect, the present invention provides a method for enhancing and/or conferring resistance of a plant or a part thereof towards at least one disease caused by a phytopathogenic fungus and/or oomycete, comprising contacting the plant, part thereof or the plant habitat with at least one bacterial strain selected from the group consisting of LAV104661, LAV104629, LAV104665, LAV104794, LAV104618, LAV104692, LAV104632, LAV104891, LAV104630, LAV104664, LAV104922, LAV59524, LAV104960, LAV104858, LAV104662, LAV104961, LAV104533, LAV58567, LAV1000636, LAV1000506, LAV1005101, LAV1001107, LAV1001081, LAV1000847, LAV1000965, LAV1000933, and functional homologs thereof, a preparation of same, a lysate thereof, an extract thereof, a broth obtained therefrom or a composition comprising same. Each possibility represents a separate embodiment of the present invention.

According to yet a further aspect, the present invention provides a method for preventing or treating a plant disease caused by a phytopathogenic fungus and/or oomycete, comprising contacting a plant or a part thereof or the plant habitat with at least one bacterial strain selected from the group consisting of LAV104661, LAV104629, LAV104665, LAV104794, LAV104618, LAV104692, LAV104632, LAV104891, LAV104630, LAV104664, LAV104922, LAV59524, LAV104960, LAV104858, LAV104662, LAV104961, LAV104533, LAV58567, LAV1000636, LAV1000506 LAV1005101, LAV1001107, LAV1001081, LAV1000847, LAV1000965, LAV1000933, and functional homologs thereof; a preparation of same, a lysate thereof, an extract thereof, a broth obtained therefrom or a composition comprising same. Each possibility represents a separate embodiment of the present invention.

Any method as is known in the art for contacting bacterial strains with a plant, a part thereof or the plant habitat can be used according to the teachings of the present invention. Typically, the plant or part thereof is contacted with a composition comprising the bacterial strains, functional homologs thereof or preparations of same. According to some embodiments, the plant can be contacted directly with the composition. According to some embodiments, the composition may be applied to the plant habitat. According to certain embodiments, the plant habitat is a growth medium, which can be a solid or a liquid growth medium. According to certain exemplary embodiments, the solid growth medium is soil. According to certain embodiments, the composition is formulated in a liquid form. According to these embodiments, the plant or part thereof may be contacted with the composition by a method selected from the group consisting of infiltration, immersion/dipping, incubation, spraying, and any combination thereof. Each possibility represents a separate embodiment of the present invention.

According to certain exemplary embodiments, the plant part is a seed. According to certain embodiments, the at least one bacterial strain according to the teachings of the invention is applied to the seed via seed coating.

According to certain additional or alternative exemplary embodiments, the plant part is a leaf According to some embodiments, the at least one bacterial strain is applied to the leaf by spraying or dusting a bacterial preparation or a composition comprising same according to the teachings of the present invention.

According to certain exemplary embodiments, the plant part is a root and contacting is performed by dipping or immersing.

According to certain embodiments, the plant or part thereof is surface sterilized prior to contacting with the bacterial preparation/composition/formulation, particularly for research applications.

In some embodiments, the bacterial preparation, compositions and/or formulations comprising same can be contacted with the plant or part thereof, for example the plant seed and/or plant aerial parts, and successful colonization can be confirmed by detecting the presence of the microbial strain within the plant. For example, after applying the composition/formulation/preparation to the seeds, high titers of the microbial strain can be detected in the roots and shoots of the plants that germinate from the seeds. In addition, significant quantities of the microbial strain can be detected in the rhizosphere of the plants. Therefore, in some embodiments, the microbial strain is applied in an amount effective to colonize the plant. The microbial strain may be colonized on the surface of the plant or within the plant tissues as an endophyte. In some embodiments, colonization of the plant can be detected, for example, by detecting the presence of the microbial strain inside the plant. This can be accomplished by measuring the viability of the microbial strain after surface sterilization of the plant portion: microbial strain colonization results in an internal localization of the microbe, rendering it resistant to conditions of surface sterilization. According to some embodiments, the microbial strain is applied in an amount effective to colonize the plant rhizosphere. The presence and quantity of the microbial strain can also be established using other means known in the art, for example, immunofluorescence microscopy using microbe-specific antibodies, or fluorescence of in situ hybridization. Alternatively, specific nucleic acid probes recognizing conserved sequences from the colonized bacterial strain can be employed to amplify a region, for example by quantitative PCR, and correlated to CFUs by means of a standard curve.

According to certain exemplary embodiments, the bacterial strain can be detectable within a target tissue of the mature cultivated plant selected from a fruit, a seed, a leaf, or a root, a portion thereof and a combination thereof. It is to be explicitly understood that the bacterial strain or the functional homolog can colonize a plant part distinct from the plant part with which said bacterial strain or functional homolog was contacted.

According to certain embodiments, the bacterial strain contacted with the plant, part thereof or the plant rhizosphere is not detected in said plant, plant part thereof and rhizosphere prior to application of said bacterial strain. According to other embodiments, the bacterial strain is naturally present in the plant, part thereof, and/or plant rhizosphere. In any case, the amount of the bacterial strain in the plant, part thereof or plant rhizosphere is higher after contacting with the strain compared to its amount prior to contacting (application).

According to other embodiments, the bacterial strain is applied (contacted), for example, on the surface of at least part of a cultivated plant, in an amount effective in conferring and/or enhancing resistance of the cultivated plant to at least one pathogenic fungus and/or oomycete and/or treating and/or preventing a disease caused by at least one pathogenic fungus and/or oomycete.

As used herein, the phrase “CFUs” or “Colony Forming Units” refers to the number of microbial cells in a defined sample (e.g., milliliter of liquid, square centimeter of surface, one seed of grain, etc.) that form colonies and thereafter numbered, on a semi-solid bacteriological growth medium.

According to certain embodiments, the bacterial strain is applied at a concentration range of from about 10² CFU/ml to about 10¹⁰ CFU/ml of the bacterial preparation or the agricultural composition. According to some embodiments, the bacterial strain is applied to seeds at a concentration range of from about 10² CFU/seed to about 10¹⁰ CFU/seed. According to certain exemplary embodiments, the bacterial strain is applied at a concentration range of from about 10⁷ CFU/ml to about 10⁸ CFU/ml of the bacterial preparation or the agricultural composition, or from about 10⁷ CFU to about 10⁸ CFU per seed.

According to certain aspects, the present invention provides a modified bacterial strain having an improved capability to confer or enhance the resistance of a plant towards pathogenic fungi and/or oomycetes, or to protect the plant from a disease caused by the pathogenic fungi and/or oomycetes, as compared to a corresponding unmodified bacterial strain. Genetic modification either of structural gene sequences or the sequences that regulate expression of said genes could be directed at the genes implicated or historically linked to fungicidal activity (some but not all being described herein). This approach could be used to improve performance of the strains disclosed in the present invention.

According to certain currently exemplary embodiments, the modified bacterial strain is a non-genetically modified organism (Non-GMO). According to certain exemplary embodiments, the modified bacterial strain is LAV58567.

According to certain further aspects, the present invention provides a method of obtaining a modified bacterial strain having a capability to confer or enhance the resistance of a plant towards pathogenic fungi and/or oomycetes, or to protect the plant from a disease caused by the pathogenic fungi and/or oomycetes, as compared to a parent unmodified bacterial strain, comprising:
(a) culturing a parent bacterial strain having a capability to confer or enhance the resistance of a plant towards pathogenic fungi and/or oomycetes, or to protect the plant from a disease caused by the pathogenic fungi and/or oomycetes under conditions suitable for expanding a population of the bacterial strain and allowing development of at least one bacterial mutant, and
(b) selecting at least one bacterial mutant resultant of step (a) for an improved capability to confer or enhance the resistance of a plant towards the pathogenic fungi and/or oomycetes, or to protect the plant from a disease caused by the pathogenic fungi and/or oomycetes,
thereby obtaining the modified bacterial strain having the improved capability to confer or enhance the resistance of a plant towards said pathogenic fungi and/or oomycetes, or to protect the plant from a disease caused by said pathogenic fungi and/or oomycetes as compared to the unmodified parent bacterial strain.

According to certain embodiments, the phytopathogenic fungus is of a genus selected from the group consisting of Fusarium, Botrytis, Erysiphe, Aspergillus, and Rhizopus. Each possibility represents a separate embodiment of the present invention.

Specific pathogenic fungi or oomycetes are known to cause dramatic crop loss due to disease symptoms which negatively affect the quality of the crop. For example, Fusarium verticillioides and Fusarium graminearum cause rot in maize (specifically stalk rot), wheat, sweet pepper, and eggplants, and head blight in wheat. Fusarium oxysporum causes sudden death syndrome (SDS) in soybeans, yellow spots in sugar beet, Panama disease in banana, and wilt in tomato, sweet pepper, eggplants, potatoes and various plants of the Cucurbitaceae family. Botrytis cinerea causes Gray Mold in tomato, sweet pepper, eggplant, potato, grapes and many other hosts. Presence of Aspergillus niger on grapes causes Black Mold and, together with Acetobacter bacteria, causes Sour rot in grapes.

According to certain embodiments, the phytopathogenic fungus is selected from the group consisting of Fusarium graminearum, Fusarium verticillioides, Fusarium oxysporum, Botrytis cinerea, Erysiphe necator, Aspergillus niger, and Rhizopus stolonifera. According to certain exemplary embodiments, the phytopathogenic fungus is selected from the group consisting of Fusarium graminearum, Fusarium verticillioides, and Botrytis cinerea. Each possibility represents a separate embodiment of the present invention.

According to certain embodiments, the oomycete is of a genus selected from the group consisting of Plasmopara, Pythium, Phytophthora, and Pseudoperonospora. Each possibility represents a separate embodiment of the present invention.

According to certain exemplary embodiments, the pathogenic fungus is Fusarium graminearum, and the bacterial strain is selected from the group consisting of LAV104794, LAV104632, LAV104664, LAV104630, LAV104891, LAV58567, LAV104922, LAV59524, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic fungus is Fusarium verticillioides, and the bacterial strain is selected from the group consisting of LAV104794, LAV104662, LAV104692, LAV104630, LAV104922, LAV58567, LAV104661, LAV104629, LAV104665, LAV104664, LAV104618, LAV104891, LAV59524, LAV104960, LAV104961, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic fungus is Botrytis cinerea, and the bacterial strain is selected from the group consisting of LAV104794, LAV104661, LAV104662, LAV104632, LAV104858, LAV104630, LAV59524, LAV104960, LAV104665, LAV104692, LAV104618, LAV104891, LAV104922, LAV104961, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic oomycete is Pythium aphanidermatum, and the bacterial strain is selected from the group consisting of LAV104794, LAV104629, LAV104665, LAV104858, LAV104664, LAV104692, LAV104630, LAV104891, LAV104922, LAV59524, LAV58567, LAV104661, LAV104662, LAV104632, LAV104618, LAV104961, LAV1000965, LAV1000847, LAV1000933, LAV1001081, LAV1001107, LAV1005101, functional homologs thereof and any combination thereof. Each possibility represents a separate embodiment of the present invention.

According to certain exemplary embodiments, the pathogenic oomycete is Pythium irregulare, and the bacterial strain is selected from the group consisting of LAV104661, LAV104662, LAV104629, LAV104665, LAV104664, LAV104630, LAV1000506, LAV1000636, LAV104632, LAV104618, LAV1000933, LAV1001081, LAV1001107, LAV1005101, functional homologs thereof and any combination thereof. Each possibility represents a separate embodiment of the present invention.

According to certain exemplary embodiments, the pathogenic oomycete is Plasmopara viticola, and the bacterial strain is selected from the group consisting of LAV59524, LAV104960, LAV104961, LAV104533, LAV58567, functional homologs thereof and any combination thereof. Each possibility represents a separate embodiment of the present invention.

The term “plant” as used herein encompasses a whole plant, a grafted plant, ancestor(s) and progeny of the plants and plant parts, including seeds, shoots, stems, roots (including tubers), rootstock, scion, and plant cells, tissues and organs. The plant or part thereof may be in any form, including suspension cultures, embryos, meristematic regions, callus tissue, leaves, gametophytes, sporophytes, pollen, and microspores. Plants to be treated according to the methods of the invention include all plants which belong to the superfamily Viridiplantae, in particular monocotyledonous and dicotyledonous plants, including a fodder or forage legume, an ornamental plant, a food crop, a tree, or a shrub.

According to certain embodiments, the plant of which the resistance/tolerance towards pathogenic fungi and/or oomycetes is enhanced, or which is being protected from the pathogenic fungi and/or oomycetes, is of a family selected from the group consisting of Actinidiaceae, Amaranthaceae, Anacardiaceae, Apiaceae, Apocynaceae, Araceae, Araliaceae, Arecaceae, Asteraceae, Begoniaceae, Brassicaceae, Cactaceae, Cannabaceae, Cannaceae, Caprifoliaceae, Caryophyllaceae, Casuarinaceae, Chenopodiaceae, Cucurbitaceae, Cyperaceae, Ericaceae, Euphorbiaceae, Fabaceae, Geraniaceae, Gesneriaceae, Ginkgoaceae, Hydrangeaceae, Iridaceae, Juglandaceae, Lamiaceae, Magnoliaceae, Malvaceae, Moraceae, Musaceae, Myrtaceae, Oleaceae, Papaveraceae, Passifloraceae, Pedaliaceae, Piperaceae, Platanaceae, Poaceae, Poaceae, Polygonaceae, Portulacaceae, Proteaceae, Punicaceae, Ranunculaceae, Rosaceae, Rubiaceae, Rutaceae, Salicaceae, Scrophulariaceae, Solanaceae, Sterculiaceae, Taxodiaceae, Theaceae, Tiliaceae, Verbenaceae, Violaceae, Vitaceae, and Zingiberaceae.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1: Sourcing of Microbial Strains with Potential Bio-Fungicidal Activity

This example describes the source from which the microbial strains were isolated prior to screening for bio-fungicidal activity. Table 1 hereinbelow presents the microbial strains described in this invention and their origin.

### Experimental Procedures

Sampling was carried out during the years 2018-2020. Source plants, soil or water samples were taken from various relevant habitats across Israel. For isolation from soil, about 1 gr soil was shaken in 20 ml phosphate buffered saline [PBS; per liter: 8 gr sodium chloride (NaCl), 0.2 gr potassium chloride (KCl), 1.42 gr disodium phosphate (Na₂HPO₄) and 0.24 gr potassium phosphate (KH₂PO₄), pH7.4] for 20 minutes at 120 rpm, and 1 ml was taken for serial dilution as described below.

For isolation from plant tissue, source plant organs (leaves, berries, roots) were removed. In the laboratory, the detached organs were immersed in sterile PBS and shaken for 30 min at 200 revolutions per minute (RPM). Thereafter, the plant organs were transferred carefully to a new 50 ml Falcon tube.

Water samples, and microbes released into the medium from the plant organs or soil, were serially diluted, and the selected dilutions were plated onto several bacteriological growth media including, but not limited to, R2G [per liter: 0.5 g proteose peptone, 0.5 g casamino acids, 0.5 g yeast extract, 0.5 g dextrose, 0.5 g soluble starch, 0.3 g dipotassium phosphate (K₂HPO₄), 0.05 g magnesium sulfate (MgSO₄·7H₂O), 0.3 g sodium pyruvate and 8 g gelrite as a gelling agent], PDA (per liter: 4 g potato extract, 20 g dextrose and 20 g agar), NA (per liter: 5 g peptone and 15 g agar as gelling agent), NYDA (per liter: 7 g peptone, 5 g yeast extract, 1 g glucose, 4 g NaCl and 13 g agar as gelling agent), and Actinomycete Isolation Agar (per liter: 0.1 g L-asparagine, 0.5 g K₂HPO₄, 0.001 g FeSO4 and 15 g agar as gelling agent).

Isolated colonies that appeared on plates after 24-72 hours of growth at 28° C. in the dark were further picked and re-isolated on a new R2G plate, before storage in an R2A broth [per liter: 0.5 g proteose peptone, 0.5 g casamino acids, 0.5 g yeast extract, 0.5 g dextrose, 0.5 g soluble starch, 0.3 g dipotassium phosphate (K₂HPO₄), 0.05 g magnesium sulfate (MgSO₄·7H₂O), 0.3 g sodium pyruvate], supplemented with 25% glycerol, at minus−80° C. Isolates were identified to the strain level by whole genome sequencing using an Illumina MiSeq sequencing platform, or to the species level by Sanger sequencing of the 16S-rRNA gene with the universal primers 16S_27F and 16S_1492R (SEQ ID NOs: 68-69; see Example 3 hereinbelow).

TABLE 1
Microbial strains described according
to some embodiments of the invention

| Microbial | | | |
|---|---|---|---|
| strain name | Origin | Plant type | Plant tissue |
| LAV104794 | Spring water | NR | NR |
| LAV104661 | Grape (Vitis vinifera) | Commercial | Phyllosphere |
| | | variety | |
| LAV104662 | Grape (Vitis vinifera) | Commercial | Phyllosphere |
| | | variety | |
| LAV104632 | Tomato (Solanum | Commercial | Rhizosphere |
| | lycopersicum) | variety | |
| LAV104629 | Spring water | NR | NR |
| LAV104665 | Wheat (Triticum | Commercial | Rhizosphere |
| | aestivum) | variety | |
| LAV104664 | Grape (Vitis vinifera) | Commercial | Phyllosphere |
| | | variety | |
| LAV104692 | Corn (Zea mays) | Commercial | Seed |
| | | variety | endosphere |
| LAV104630 | Spring water | NR | NR |
| LAV104618 | Soil | NR | NR |
| LAV104891 | Spring water | NR | NR |
| LAV104922 | Spring water | NR | NR |
| LAV59524 | Wheat (Triticum | Commercial | Rhizosphere |
| | aestivum) | variety | |
| LAV104960 | Soil | NR | NR |
| LAV104961 | Soil | NR | NR |
| LAV104858 | Corn (Zea mays) | Commercial | Rhizosphere |
| | | variety | |
| LAV104533 | Marine sponge | NR | NR |
| | (Spheciospongia | | |
| | vagabunda) | | |
| LAV1000506 | soil | NR | NR |
| LAV1000636 | soil | NR | NR |
| LAV1000965 | Grape (Vitis vinifera) | Commercial | Phyllosphere |
| | | variety | |
| LAV1000847 | soil | NR | NR |
| LAV1000933 | soil | NR | NR |
| LAV1001081 | Lentil-like vetchling | Wild plant | Root |
| | (Lathyrus lentiformis) | | endosphere |
| LAV1001107 | Red baron | Wild plant | Root |
| | (Imperata cylindrica) | | endosphere |
| LAV1005101 | Bird's-foot trefoil | Wild plant | Root |
| | (Lotus peregrinus) | | endosphere |

Table 1. NR- not relevant

### Example 2: Directed Evolution for Improving Phyllosphere Survival and Biocontrol Efficacy of a Strain with Biocontrol Activity Against Downy Mildew in Grapes

Directed evolution mimics natural evolution via iterative cycles of diversity generation and functional selection or screening to isolate evolved mutants with desirable phenotypes.

Isolate LAV55906 was isolated from an agricultural soil in Israel and identified as Lysobacter capsici. Biocontrol activity was assessed against the grape pathogen Plasmopara viticola and found to be similar to that of Lysobacter capsici strain AZ78, which can be combined with copper to effectively control Plasmopara viticola on grapevine (Puopolo et al., Microbiol. Research 169, 2014). However, the survival of the strain LAV55906 on the plant leaves has been found to be rather limited (up to 5-6 days).

An accelerated evolution process was initiated, directed to selection of the longest surviving colonies on the vine grape phyllosphere, followed by enrichment of the new population and assessment of the efficacy against the pathogen Plasmopara viticola causing Downy Mildew in grapes.

In brief, the longest surviving colony was selected, by stamping bacteria-inoculated leaves on selective agar R2A plates containing 50 μg/ml kanamycin, utilizing the natural kanamycin resistance of the isolate. The process was repeated every day for 16 days after inoculation, until a point that no colonies appeared. The last single colony was then propagated and tested for longer survival, and then included in the efficacy assays as described in examples 6 and 7 hereinbelow. The process was repeated several times, at which point the best performing isolate after 16 days (LAV58567) was selected.

Genomic sequences were extracted for the novel isolate LAV58567 and the parent isolate LAV55906. Sequence comparison revealed 1 insertion (in a non-coding region), 5 intergenic mutations, 10 synonymous mutations and 3 missense mutations, as detailed in Table 2 hereinbelow.

Table 3 hereinbelow shows the comparison of disease severity reduction by the parental strain LAV55906 and by the evolved strain LAV58567 as measured in a whole plant assay (as described in Example 7 hereinbelow) by application of the pathogen 6 days after the bacterial treatments.

TABLE 2
Genomic comparison of parent LAV55906 and
evolved LAV58567

| | | | | Altered | | |
|---|---|---|---|---|---|---|
| Mutation | | Altered | | Amino | | |
| Position | | Nucleotides | | Acids | | |
| | nu- | Parent | Evolved | | Evolved | |
| Scaf- | cleo- | nu- | 58567 | Parent | 58567 | |
| fold | tide | cleo- | nucleo- | amino | amino | Mutation |
| # | number | tides | tides | acids | acids | type |
| 2 | 77322 | T | C | R | R | synonymous |
| 2 | 77338 | T | C | F | F->L | missense |
| 5 | 220867 | A | G | R | R | synonymous |
| 5 | 220881 | A | G | L | L | synonymous |
| 8 | 157672 | GGG | AGT | NR | NR | intergenic |
| 8 | 157789 | A | G | NR | NR | intergenic |
| 10 | 171579 | GACA | AACG | VV | VV | synonymous |
| 11 | 98026 | G | A | NR | NR | intergenic |
| 11 | 98062 | G | A | NR | NR | intergenic |
| 15 | 129942 | A | G | L | L | synonymous |
| 28 | 7486 | G | C | G | G->R | missense |
| 29 | 20832 | C | CTTCCCGA | NR | NR | insertion |
| | | | | | | (non- |
| | | | | | | coding) |
| 40 | 24849 | C | T | NR | NR | intergenic |
| 41 | 1874 | G | C | A | A->P | missense |
| 49 | 17742 | C | T | E | E | synonymous |
| 70 | 123 | C | T | N | N | synonymous |
| 70 | 147 | G | C | T | T | synonymous |
| 97 | 78 | C | G | V | V | synonymous |
| 97 | 84 | G | C | V | V | synonymous |

Table 2. List of mutations between strains by scaffold number and nucleotide position within scaffold; NR - not relevant.

TABLE 3
Efficacy comparison of parent strain
LAV55906 and evolved strain LAV58567

| Treatment | Disease severity | P value |
|---|---|---|
| Untreated | 9.5 | |
| LAV55906 | 4.1 | 0.002* |
| LAV58567 | 2.6 | 0.0001* |
| | | 0.1183* |

Table 3. Efficacy of evolved isolate as measured by significantly reduced disease severity. Disease was estimated as described for the detached leaf assay (Example 5 hereinbelow). P-value was determined by a paired sample 2-tailed t-test. *—tested against untreated control; **—tested against parent LAV55906.

### Example 3: Microbial Strain Characterization by 16S-rRNA

The microbial strains of the present invention have been further characterized based on their 16S-rRNA sequence. Without wishing to be bound by any theory or mechanism of action, any strain with a significantly homologous 16S-rRNA sequence is likely to exhibit the same functional properties, and therefore can be used as a fungicide as described in this invention.

### Experimental Procedures

16S-rRNA sequences were obtained by either:
1) Polymerase Chain Reaction (PCR; Mullis, K. B., et al. 1987. Process for amplifying, detecting, and/or-cloning nucleic acid sequences. U.S. Pat. No. 4,683,195) using the universal primers 16S_27F (AGAGTTTGATCMTGGCTCAG, SEQ ID NO:68) and 16S_1492R (TACGGYTACCTTGTTACGACTT, SEQ ID NO:69) (Eden, P. A., et al. 1991. Phylogenetic analysis of Aquaspirillum magnetotacticum using polymerase chain reaction-amplified 16S rRNA-specific DNA. Int. J. Syst. Bacteriol. 41:324-325) followed by Sanger sequencing (Sanger, F., and Coulson, A. R. 1975. A rapid method for determining sequences in DNA by primed synthesis with DNA polymerase. J. Mol. Biol. 94:441-448) of the amplified fragments using the primers 16S_27F and 16S_1492R, and the additional primers 16S_5151F (GTGCCAGCMGCCGCGGTAA, SEQ ID NO:70) and 16S_970R (CCGTCAATTCMTTTRAGTTT, SEQ ID NO:71).
2) Extraction of 16S-rRNA sequences from the assembly of microbial strains genome sequences (Applicant proprietary pipeline) using the MOTHUR tool (a bioinformatics tool for analyzing 16S-rRNA gene sequences; Schloss, P. D., Westcott, S. L., Ryabin, T., Hall, J. R., Hartmann, M., Hollisteret, E. B., al. 2009. Introducing mothur: open-source, platform-independent, community-supported software for describing and comparing microbial communities. Appl. Environ. Microbiol. 75:7537-7541) and SILVA database (a comprehensive on-line resource for quality checked and aligned ribosomal RNA sequence data; Pruesse, E., Quast, C., Knittel, K., Fuchs, B. M., Ludwig, W., Peplies, J., and Glöckner, F. O. 2007. SILVA: a comprehensive online resource for quality checked and aligned ribosomal RNA sequence data compatible with ARB. Nucl. Acids Res. 35:7188-7196) as a reference.

Obtained 16S-rRNA sequences were clustered (grouped) to Operational Taxonomic Units (OTUs) using nucleotide-based local alignment search tool (BLASTN; Altschul, S., Gish, W., Miller, W., Myers, E. W., and Lipman, D. J. 1990. Basic local alignment search tool. J. of Mol. Biol. 215:403-410).

TABLE 4
Bacterial strain organism and 16S-rRNA SEQ ID NOs.

| | | 16S rRNA | Homologous |
|---|---|---|---|
| Isolate ID | | SEQ ID | sequences |
| Number | Organism | NOs. | (Over 97%) |
| LAV104661 | Erwinia sp. | 14 | |
| LAV104629 | Pantoea sp. | 1 | |
| LAV104665 | Pseudomonas sp. | 15 | |
| LAV104794 | Pseudomonas sp. | 16 | 6 |
| LAV104618 | Ensifer sp. | 17 | |
| LAV104692 | Pantoea sp. | 18 | |
| LAV104632 | Bacillus sp. | 2 | |
| LAV104891 | Acinetobacter sp. | 19 | 22 |
| LAV104630 | Enterobacter sp. | 20 | |
| LAV104664 | Pseudomonas sp. | 21 | |
| LAV104922 | Acinetobacter sp. | 22 | 19 |
| LAV59524 | Bacillus sp. | 23 | |
| LAV104960 | Streptomyces sp. | 24 | |
| LAV104858 | Pseudomonas sp. | 3 | |
| LAV104662 | Sphingobium sp. | 4 | |
| LAV104961 | Streptomyces sp. | 25 | |
| LAV104533 | Actinokineospora sp. | 26 | |
| LAV58567 | Lysobacter sp. | 5 | |
| LAV1000636 | Pseudomonas sp. | 6 | 16 |
| LAV1000506 | Gordonia sp. | 7 | |
| LAV1000965 | Pseudomonas sp. | 8 | |
| LAV1000847 | Pseudomonas sp. | 9 | |
| LAV1000933 | Paenibacillus sp | 10 | |
| LAV1001081 | Rahnella sp. | 11 | |
| LAV1001107 | Pseudomonas sp. | 12 | |
| LAV1005101 | Pseudoxanthomonas sp | 13 | |

Table 4: List of bacterial strains of the invention, and their respective 16S-rRNA sequences, as disclosed in the Sequence Listing herein. Some 16S-rRNA sequences showed homology higher than 97% global identity to sequences of other strains of the invention, as detailed in the rightmost column.

### Example 4: Clustering of Microbial Strains Using Strain-Specific Genomic Markers

### Experimental Procedures

DNA fragments with lengths ranging from 200 bp to 1000 bp from genomes of the microbial strains of certain embodiments of this invention were screened against the NCBI bacteria genome refseq nucleotide database, using NCBI local alignment tool BLASTN (NCBI-blast-2.10.0+). Criteria for declaring a microbial strain-specific marker were finding no refseq sequences with at least 90% coverage and with at least 95% local sequence identity. Up to 3 microbial strain-specific genomic markers were selected for each microbial strain described in this invention.

TABLE 5
Genomic Marker SEQ ID NOs. per strain

| | | | Genomic Marker |
|---|---|---|---|
| | | | Sequences length |
| | Genomic | | (nucleotides), |
| Microbial | Marker | Number of | concordant with |
| strain | SEQ ID | Microbial strain- | Marker |
| number | NOs. | specific markers | SEQ ID NOs.* |
| LAV104533 | NA | | |
| LAV104618 | 75-77 | 3 | 921; 911; 863 |
| LAV104629 | 27-28 | 2 | 927; 328 |
| LAV104630 | 84-85 | 2 | 203; 200 |
| LAV104632 | 29 | 1 | 233 |
| LAV104661 | 67-68 | 2 | 827; 769 |
| LAV104662 | 33-35 | 3 | 992; 932; 672 |
| LAV104664 | 86-88 | 3 | 896; 766; 747 |
| LAV104665 | 69-71 | 3 | 413; 389; 323 |
| LAV104692 | 78-80 | 3 | 695; 516; 455 |
| LAV104794 | 72-74 | 3 | 601; 450; 302 |
| LAV104858 | 30-32 | 3 | 779; 666; 587 |
| LAV104891 | 81-83 | 3 | 685; 362; 284 |
| LAV104922 | 89-91 | 3 | 292; 286; 206 |
| LAV104960 | NA | | |
| LAV104961 | 95-97 | 3 | 876; 767; 760 |
| LAV58567 | 36-38 | 3 | 894; 784; 751 |
| LAV59524 | 92-94 | 3 | 726; 670; 656 |
| LAV1000506 | 40-42 | 3 | 551; 538; 538 |
| LAV1000636 | 39 | 1 | 621 |
| LAV1000965 | 43-47 | | |
| LAV1000847 | 48-50 | | |
| LAV1000933 | 51-55 | | |
| LAV1001081 | 56 | | |
| LAV1001107 | 57-61 | | |
| LAV1005101 | 62-66 | | |

Table 5: *NA - Not Available (no genomic marker sequences were found meeting the criteria detailed above).

### Example 5: Screening and Evaluation of Potential Fungal Biocontrol Activity of Bacterial Isolates

Microbial strains obtained as described in Examples 1 and 2 hereinabove were screened for their ability to suppress the mycelial growth of Fusarium graminearum, Fusarium verticillioides, Botrytis cinerea and Pythium aphanidermatum by in vitro dual culture assays on potato dextrose agar (PDA).

### Experimental Procedures

Fungal cultures were routinely maintained on PDA medium plates. Isolated microbial strains were grown in R2A medium (as described in Example 1 hereinabove) at 28° C. for 48 hours. Cell pellets were then collected by centrifugation at 10,000 rpm at room temperature for 5 minutes and re-suspended in 2 ml of sterile PBS (as described in Example 1 hereinabove).

A PDA agar plug from a fungal culture was placed in the center of a PDA Petri dish (bottom-up) and 5 microliters of a bacterial cell culture were plated at 2.5 cm distance from the center. Four different bacteria were included in each plate. Each combination of pathogenic fungus/bacteria was replicated 3 times and plates were randomly placed in the dark and incubated at 25° C. for 5 days. As negative controls, 3 Petri dishes were inoculated only with the fungal cultures. The radial growth of the fungus mycelium colony towards a specific bacterial colony was measured (mm) and the average inhibition was calculated relative to the negative control.

Table 6 shows the effects of the microbial strains on the growth of the tested fungal pathogens in the in vitro dual culture assays.

TABLE 6
in vitro inhibition of fungal growth by the microbial strains

| | Fungal Species | | | | | | | |
|---|---|---|---|---|---|---|---|---|
| Microbial | Fusarium | | Fusarium | | Botrytis | | Pythium | |
| strain | graminearum | | verticillioides | | cinerea | | aphanidermatum | |
| LAV104794 | 5 | (0.002) | 2.75 | (0.05) | 8.5 | (0.02) | 8.25 | (0.007) |
| LAV104661 | 0 | | 0.5 | (0.06) | 0.75 | (0.01) | 0 | |
| LAV104662 | 0 | | 0.75 | (0.02) | 0.8 | (0.05) | 0 | |
| LAV104632 | 1 | (0.04) | 0 | | 1.25 | (0.02) | 0 | |
| LAV104629 | 0 | | 0.75 | (0.1) | 0 | | 1.25 | (0.04) |
| LAV104665 | 0 | | 0.75 | (0.1) | 7.25 | (0.06) | 8 | (0.03) |
| LAV104858 | 0 | | 0 | | 0.75 | (0.05) | 4.5 | (0.04) |
| LAV104664 | 0.75 | (0.04) | 1 | (0.08) | 0 | | 23.75 | (0.003) |
| LAV104692 | 0 | | 1.25 | (0.01) | 1 | (0.07) | 3 | (0.03) |
| LAV104630 | 1.3 | (0.03) | 1.25 | (0.05) | 4.25 | (0.03) | 9.25 | (0.005) |
| LAV104618 | 0 | | 0.25 | (0.1) | 1 | (0.06) | 0.5 | (0.1) |
| LAV104891 | 0.3 | (0.05) | 1.25 | (0.1) | 4.25 | (0.08) | 9.75 | (0.007) |
| LAV104922 | 0.75 | (0.08) | 1 | (0.05) | 4 | (0.07) | 8.25 | (0.01) |
| LAV59524 | 0.75 | (0.09) | 1 | (0.08) | 3.5 | (0.01) | 7.75 | (0.03) |
| LAV104960 | 0 | | 0.25 | (0.1) | 9.75 | (0.01) | 0 | |
| LAV104961 | 0 | | 0.4 | (0.09) | 0.25 | (0.2) | 1.75 | (0.1) |
| LAV104533 | NA | | NA | | NA | | NA | |
| LAV58567 | 6 | (0.01) | 5.5 | (0.02) | 0 | | 3 | (0.04) |
| LAV1000965 | 1 | (0.05) | 1.3 | (0.006) | 3.3 | (0.005) | 0.3 | (0.2) |
| LAV1000847 | 1 | (0.07) | 1 | (0.05) | 1 | (0.04) | 3 | (0.003) |
| LAV1000933 | 1 | (0.1) | 1 | (0.04) | 1 | (0.1) | 3 | (0.003) |
| LAV1001081 | 3 | (0.007) | 1.5 | (0.002) | 0.16 | (0.1) | 0.5 | (0.1) |
| LAV1001107 | 0.67 | (0.2) | 1 | (0.07) | 0.33 | (0.1) | 1 | (0.15) |
| LAV1005101 | 0.67 | (0.1) | 1 | (0.05)(0.1) | 0.33 | (0.1) | 0.5 | (0.1) |

Table 6: The radial mycelial growth of the fungi towards the antagonistic bacteria (Rᵢ) and that on a control plate (R_{c}) were measured and the mycelial growth inhibition (mm) was calculated as the difference R_{c}−Rᵢ; Numbers in parentheses denote p-value (t-test, significant at P≤0.05). NA—Not Available.

### Example 6: In Vivo Inhibition of Downy Mildew Disease Development in Detached Grapevine Leaves

### Experimental Procedures

Isolated microbial strains were grown in R2A medium (as described in Example 1 hereinabove) at 28° C. for 24 hours. Cell pellets were then collected by centrifugation at 10,000 rpm at room temperature for 5 minutes and re-suspended in 10 ml of sterile PBS. Cell density of each microbial suspension was determined (O.D. 600). Cell concentration was determined by plating serial dilutions (in PBS) on R2G plates, counting and calculating Colony Forming Units (CFUs) after 2 days of growth at 28° C. in the dark.

Inhibition of Downy Mildew disease caused by Plasmopara viticola was evaluated using a detached leaf assay essentially as described by Prajongjai et al. (S. Afr. J. Eno. Vitic 35:43; 2014). Leaves of similar age and area were taken from plants grown in a greenhouse at Evogene Farm (Israel) from two primary grape (V. vinifera) varieties: Cabernet and Merlot.

The leaves were surface-disinfected with 0.09% (w/v) sodium hypochlorite and 0.01% (v/v) Tween 20, rinsed with sterile distilled water, and placed abaxial surface up on moist filter paper in a Petri dish.

Bacterial suspensions were sprayed on the leaf surface until run-off: about 1 ml suspension/leaf corresponding to a bacterial concentration of 10⁷-10⁸ CFU/ml, (4 leaf repeats per concentration), and allowed to dry before pathogen application.

Plasmopara viticola sporangia were collected from Downy Mildew-infected V. vinifera leaves, routinely maintained in a controlled growth chamber, by flooding the leaf surface with water. The sporangial suspension concentration was estimated using a hemocytometer and adjusted to 1×10⁴ sporangia/ml.

To examine the anti-fungal activity of the bacteria strains, the sporangial suspensions were sprayed onto the abaxial leaf surface of each detached leaf described above until evenly wet. Leaves with no prior bacterial treatment (fungi only) served as the normal disease development control for the experiment. Petri dishes were incubated at 24° C., 16 h photoperiod for 7 days. Infected leaves were then transferred into an incubator at 19° C. at 100% relative humidity (RH) overnight for enhanced sporulation for disease estimation. Disease severity of infected leaf area was classified into 3 classes: 0=no sporulation detected; 1=10-20% infected leaf area; 2=30-50% infected leaf area; 3=>50% infected leaf area. Percent disease inhibition afforded by the bacterial treatment was calculated relative to the untreated leaves' score.

Table 7 shows the microbial strains that significantly decreased Downy Mildew development in grapevine detached leaves compared to the non-treated control (Fisher Exact test, p-value<0.2).

TABLE 7
Microbial strains inhibiting Downy Mildew development
in detached leaves of grapevine plants

| Microbial | | |
|---|---|---|
| strain number | % Inhibition | P value |
| LAV59524 | 70 | 0.004 |
| LAV104960 | 100 | 0.004 |
| LAV104961 | 100 | 0.004 |
| LAV104533 | 100 | 0.001 |
| LAV58567 | 100 | 0.001 |

Table 7: Inhibition of Downy Mildew development on detached leaves calculated according to the formula (DS_{c}−DSₜ)/DS_{c}×100, wherein DS_{c}=disease score in untreated (control) leaves; DSₜ=disease score in leaves treated with the indicated microbial strain.

### Example 7: In Vivo Inhibition of Downy Mildew Disease in Intact Grapevine Plants

Bacterial strains which successfully passed the detached leaf assay described in Example 6 (Table 7) hereinabove were evaluated for antifungal activity on whole grapevine plants in a greenhouse-controlled environment.

Cuttings were taken from grapevines containing four or five nodes and four or more internodes on each section. A clean cut was made straight across just below a leaf node, and the basal end planted in gardening soil. Young plants with up to two shoots were used for the whole plant assays. Leaves of similar age were chosen and marked, and the abaxial side of each leaf was treated with a bacterial suspension followed by pathogen application as described in Example 6 hereinabove.

In this experiment the pathogen was applied 24 hours after the bacteria application for all isolates except for isolate LAV58567. For the latter isolate, the pathogen was applied after 6 days, since the survival of this isolate was proven to be longer (Example 2 hereinabove).

The isolates were tested on 5 plants. Two leaves on each plant were treated, one with a bacterial suspension at 10⁷ CFU/ml and the other at 10⁸ CFU/ml.

After infection, the plants were kept overnight in a mist chamber at 19° C., 100% RH in the dark and then transferred to the greenhouse at 22-24° C., 50-60% RH, 16 hours of light.

After 7 days, treated leaves were cut and placed on moist filter paper in a Petri dish and transferred into an incubator at 19° C. at 100% RH overnight for enhanced sporulation. Disease was estimated as described for the detached leaf assay (Example 6 hereinabove). Percent disease inhibition afforded by the bacterial treatment was calculated relative to the untreated leaves' score.

Table 8 shows significantly decreased Downy Mildew development in treated leaves of intact grapevine plants compared to the non-treated control (Fisher Exact test, p-value<0.2).

TABLE 8
Microbial strains inhibiting Downy Mildew disease
development in leaves of intact grapevine plants

| Microbial | | |
|---|---|---|
| strain number | % Inhibition | P value |
| LAV59524 | 80 | 0.0003 |
| LAV104960 | 80 | 0.004 |
| LAV104961 | 80 | 0.0003 |
| LAV104533 | 85 | 0.002 |
| LAV58567 | 95 | 0.001 |

Table 8: Inhibition of Downy Mildew development on leaves of intact grapevine plants calculated according to the formula (DS_{c}−DSₜ)/DS_{c}×100, wherein DS_{c}=disease score in untreated (control) leaves; DSₜ=disease score in leaves treated with the indicated microbial strain. Numbers in parentheses denote concentration of bacterial strain applied.

Isolate LAV58567 was also tested in a greenhouse plant assay against 3 different isolates of Plasmopara viticola (courtesy of the SAGEA Centro di Saggio s.r.l, Cuneo, Italy) with resistance to prominent chemical fungicide compounds (mefenoxam, cyazofamid and cymoxanil, respectively). The pathogen was applied 1 day after bacteria application. Effects of LAV58567 on disease development were compared to the effects of the three fungicide compounds described above after 2 weeks.

Table 9 compares the efficacy of the different treatments on Downy Mildew development, calculated as the percent reduction of disease severity in the treatments compared to the negative control.

TABLE 9
Inhibition of Downy Mildew disease development caused by fungicide-resistant
Plasmopara viticola isolates in leaves of intact grapevine plants

| | Efficacy on disease severity | | | |
|---|---|---|---|---|
| | | Ranman TOP | Twingo | Ridomil Gold |
| | | (AI-Cyazofamid | (AI-Cymoxanil | (AI- Mefenoxam |
| Pathogen strain | LAV58567 | 160 g/L) | 4%) | 40%) |
| Cymoxanil | 79% ^{b} | | 2.6% ^{c} | 89% ^{a} |
| resistant | | | | |
| Mefenoxam | 75% ^{b} | 88% ^{a} | | 3.4% ^{c} |
| resistant | | | | |
| Cyazofamide | 76.2% ^{b} | 3.6% ^{c} | 91% ^{a} | |
| resistant | | | | |
| Susceptible | 68% ^{b} | | 83.4% ^{a} | 89% ^{a} |

Table 9: Reduction of Downy Mildew disease severity was calculated according to Abbott's formula: (X−Y)/X×100 wherein X is disease severity in the control, and Y is disease severity in the treatment. Letters next to values denote statistically significant groupings (P=0.05, Student-Newman-Keuls). AI—active ingredient in product.

### Example 8: Vineyard Trial for Inhibition of Downy Mildew Development

Candidate isolate LAV58567 was tested over two consecutive years in commercial vineyards in northeast and northwest Italy with endemic Downy Mildew infection. The strain was tested as a dried culture re-dissolved in water at a concentration of 2 g/L Four to six consecutive applications were carried out at 7-10 day intervals.

Plant culturing conditions (soil type and fertilization) were uniform for all plots, and conformed to local viticultural practices. The design and layout of the trials was performed according to EPPO Standard PP 1/181 (Conduct and reporting of efficacy evaluation trials).

For testing Downy Mildew development, disease severity and incidence were evaluated from 100 leaves or 50 bunches from each one of the 4 plots that were set for each treatment, one week after each application. Efficacy of treatment was compared to a standard chemical positive control, biological positive control and untreated negative control. Two tests were performed in each of the two years.

TABLE 10
LAV58567 inhibition of Downy Mildew disease development in leaves
and bunches of grapevine plants in commercial vineyards

| | 2020 | | 2021 | |
|---|---|---|---|---|
| | Efficacy | Efficacy on | Efficacy | Efficacy on |
| Treatment | on leaves | bunches | on leaves | bunches |
| LAV58567 | 54.5% ^{c} | 72.5% ^{b} | 43% ^{c} | 44% ^{c} |
| Chemical | 93% ^{a} | 98% ^{a} | 97% ^{a} | 99% ^{a} |
| control | | | | |
| Organic | 70% ^{b} | 81% ^{b} | 81% ^{b} | 63% ^{b} |
| control | | | | |

Table 10: Reduction of Downy Mildew disease severity was calculated according to Abbott's formula: (X−Y)/X×100 where X is disease severity in the untreated negative control, and Y is disease severity in the treatment. Values are averages of 2 trials/year. Letters next to values denote statistically significant groupings (P=0.05, Student-Newman-Keuls).

### Example 9: Inhibition of Pythium Seedling Wilt in Corn Seedling Assay

Pythium aphanidermatum and Pythium irregulare were grown on PDA plates for 48 h at 25° C. 1 cm diameter discs were cut from the culture edge to inoculate 100 g autoclaved pearl millet seeds with the addition of 30 ml sterile water. The inoculated seeds were incubated at 25° C. for 6 days and ground to a fine mixture. 1 g of the mixture was used to inoculate 200 g of planting soil mixture [Peat (Kekila) and perlite (1:2, v/v)]. Corn seeds (Supersweet) were then planted in the mixture in trays. For a positive control, one set of corn seeds was planted in uninoculated soil mixture. Each one of two repeat experiments included 24 seeds per treatment. One ml of Bacteria solution (10⁸ CFU/ml) was added at planting time. One ml of sterile water was added to corn seeds for the mock treatment (untreated control).

After 10 days from planting, seedling emergence was recorded and the percentage was calculated, and compared to that of the untreated control.

TABLE 11
Microbial strains that significantly decrease
Pythium seedling wilt in corn

| | Pythium aphanidermatum | | Pythium irregulare | |
|---|---|---|---|---|
| | % | | % | |
| Microbial | Germination | | Germination | |
| strain number | Improvement | P value | Improvement | P value |
| LAV104794 | 97 | 0.001 | NA | NA |
| LAV104661 | 69 | 0.005 | 74 | 0.04 |
| LAV104662 | 76 | 0.001 | 60 | 0.05 |
| LAV104632 | 72 | 0.002 | 70 | 0.08 |
| LAV104629 | 50 | 0.03 | 100 | 0.009 |
| LAV104665 | 83 | 0.006 | 95 | 0.002 |
| LAV104858 | 86 | 0.05 | NA | NA |
| LAV104664 | 93 | 0.001 | 35 | 0.05 |
| LAV104692 | 92 | 0.03 | NA | NA |
| LAV104630 | 50 | 0.03 | 48 | 0.05 |
| LAV104618 | 63 | 0.07 | 78 | 0.09 |
| LAV104891 | 75 | 0.06 | NA | NA |
| LAV104922 | 100 | 0.005 | NA | NA |
| LAV1000506 | NA | NA | 55 | 0.05 |
| LAV1000636 | NA | NA | 47 | 0.04 |
| LAV1000965 | 55 | 0.02 | NA | NA |
| LAV1000847 | NA | NA | 75 | 0.001 |
| LAV1000933 | 45 | 0.04 | 63 | 0.007 |
| LAV1001081 | NA | NA | 42 | 0.04 |
| LAV1001107 | NA | NA | 54 | 0.05 |
| LAV1005101 | NA | NA | 39 | 0.07 |

Table 11: Inhibition of Pythium seedling wilt in corn seedlings. The improvement of germination compared to the untreated control was calculated according to the following formula: 00 improvement=(Germination in treatment-germination in negative control)/(Germination in positive control-germination in negative control)*100, wherein “positive control” are seedlings germinated without the pathogen and “negative control” are seedlings germinated in the presence of the pathogen without any inhibitory treatment. P-value was calculated using Fisher test. NA—not available

### Example 10: Function-Based Clustering of Microbial Strains

Reduction of plant disease symptoms by application of a microbial strain according to certain embodiments of the present invention is indicative of specific functional properties of the microbial strains. These functional properties contribute to plant tolerance against a disease caused either by a fungus or an oomycete when the microbial strains are present in/on the plant. Microorganisms are known to produce lytic enzymes, especially chitinases, alpha- and beta glucanases and xylanases (CHIs), which hydrolyze chitin, a major component of fungal cell walls, and/or cellulose, the major component of oomycete cell walls. Bacteria producing lytic enzymes are therefore an alternative strategy for controlling phytopathogens. Such bacteria may be clustered according to their lytic properties.

### Xylanase Activity

To detect xylanase production ability, 10 microliters of an overnight culture of each one of the microbial strains are spotted in 8 replicates on 0.1% xylan agar medium (Composition: yeast extract 3.0 g/L, peptone 1.5 g/L, NaCl 3.5 g/L, NaNO₃ 1.0 g/L, KH₂PO₄ 1.0 g/L, MgSO₄·7H₂O 0.3 g/L, Agar 20 g/L, and 0.1% beechwood xylan) plates (pH 5.5). Plates are incubated at 28±2° C. for 72 h. All the plates are stained with 0.5% Congo red dye for about half an hour and are then de-stained using 1 M NaCl solution at room temperature. Xylanase activity is indicated by zones of clearance scored by visual estimate. (Bushra K & Nazish M A 2016, Optimization of fermentation media and growth conditions for microbial xylanase production. 3 Biotech 6:122).

### Cellulolytic Activity

For screening of bacteria producing cellulolytic enzymes, single colonies are inoculated onto Mandels and Reese medium (Mandels M, Reese E T. Induction of cellulase in Trichoderma viride as influenced by carbon sources and metals. Journal of Bacteriology. 1957; 73 (2):269-278) containing carboxymethyl cellulose sodium salt [KH₂PO₄ 2.0 g/L; (NH₄)₂SO₄ 1.4 g/L; MgSO₄·7H₂O 0.3 g/L, CaCl₂) 0.3 g/L, yeast extract 0.4 g/L, FeSO₄·7H₂O 0.005 g/L; MnSO₄, 0.0016 g/L; ZnCl₂, 0.0017 g/L, CoCl₂ 0.002 g/L, carboxymethyl cellulose sodium (CMC-Na) 5.0 g/L, and agar 15.0 g/L; pH 5.0]. After incubation at 28° C. for 48 h, all plates are stained with 1% (w/v) Congo-red solution for 15 min and discolored with 1 M NaCl for 15 min. Degradation zones are visible around the bacteria, showing hydrolyzation of CMC by the inoculated strains.

### Chitinolytic Activity

For screening of chitinase producing bacteria, an agar solidified medium amended with colloidal chitin [Na2HPO4 6 g/L, KH2PO4 3 g/L, NH4Cl 1 g/L, NaCl 0.5 g/L, yeast extract 0.05 g/L, agar 15 g/L, and colloidal chitin 1% (w/v)] is used. Colonies showing clearance zones on a cream-colored background are considered chitinase-producing bacteria.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A bacterial preparation comprising a plurality of at least one isolated bacterial strain or a functional homolog thereof at a concentration which exceeds that found in nature, wherein the isolated bacterial strain is selected from the group consisting of:
(1) strain LAV58567, the strain being selected from the group consisting of
a. a strain deposited under Accession Number 43954 at NCIMB;
b. a strain comprising a 16S-rRNA sequence comprising the nucleic acid sequence set forth in SEQ ID NO:5;
c. a strain comprising at least one genomic marker comprising the nucleic acid sequence set forth in any one of SEQ ID NOs:36-38;
and any combination thereof,
(2) strain LAV104662, the strain being selected from the group consisting of
a. a strain deposited under Accession Number 43955 at NCIMB;
b. a strain comprising a 16S-rRNA sequence comprising the nucleic acid sequence set forth in SEQ ID NO:4;
c. a strain comprising at least one genomic marker comprising the nucleic acid sequence set forth in any one of SEQ ID NOs:33-35;
and any combination thereof,
(3) strain LAV104858, the strain being selected from the group consisting of
a. a strain deposited under Accession Number 43956 at NCIMB;
b. a strain comprising a 16S-rRNA sequence comprising the nucleic acid sequence set forth in SEQ ID NO:3;
c. a strain comprising at least one genomic marker comprising the nucleic acid sequence set forth in any one of SEQ ID NOs:30-32;
and any combination thereof,
(4) strain LAV104632, the strain being selected from the group consisting of
a. a strain deposited under Accession Number 43957 at NCIMB;
b. a strain comprising a 16S-rRNA sequence comprising the nucleic acid sequence set forth in SEQ ID NO:2;
c. a strain comprising at least one genomic marker comprising the nucleic acid sequence set forth in SEQ ID NO:29;
and any combination thereof,
(5) strain LAV104629, the strain being selected from the group consisting of
a. a strain deposited under Accession Number 43958 at NCIMB;
b. a strain comprising a 16S-rRNA sequence comprising the nucleic acid sequence set forth in SEQ ID NO:1;
c. a strain comprising at least one genomic marker comprising the nucleic acid sequence set forth in any one of SEQ ID NOs:27-28;
and any combination thereof,
(6) strain LAV1000636, the strain being selected from the group consisting of
a. a strain deposited under Accession Number 43964 at NCIMB;
b. a strain comprising a 16S-rRNA sequence comprising the nucleic acid sequence set forth in SEQ ID NO:6;
c. a strain comprising at least one genomic marker comprising the nucleic acid sequence set forth in SEQ ID NO:39;
and any combination thereof,
(7) strain LAV1000506, the strain being selected from the group consisting of
a. a strain deposited under Accession Number 43965 at NCIMB;
b. a strain comprising a 16S-rRNA sequence comprising the nucleic acid sequence set forth in SEQ ID NO:7;
c. a strain comprising at least one genomic marker comprising the nucleic acid sequence set forth in any one of SEQ ID NOs:40-42;
and any combination thereof,
(8) strain LAV1005101, the strain being selected from the group consisting of:
a. a strain deposited under Accession Number 43959 at NCIMB;
b. a strain comprising a 16S-rRNA sequence comprising the nucleic acid sequence set forth in SEQ ID NO:13;
c. a strain comprising at least one genomic marker comprising the nucleic acid sequence set forth in any one of SEQ ID NOs:62-66;
and any combination thereof,
(9) strain LAV1001107, the strain being selected from the group consisting of:
a. a strain deposited under Accession Number 43960 at NCIMB;
b. a strain comprising a 16S-rRNA sequence comprising the nucleic acid sequence set forth in SEQ ID NO:12;
c. a strain comprising at least one genomic marker comprising the nucleic acid sequence set forth in any one of SEQ ID NOs:57-61;
and any combination thereof,
(10) strain LAV1001081, the strain being selected from the group consisting of:
a. a strain deposited under Accession Number 43961 at NCIMB;
b. a strain comprising a 16S-rRNA sequence comprising the nucleic acid sequence set forth in SEQ ID NO:11;
c. a strain comprising at least one genomic marker comprising the nucleic acid sequence set forth in SEQ ID NO:56;
and any combination thereof,
(11) strain LAV1000847, the strain being selected from the group consisting of:
a. a strain deposited under Accession Number 43962 at NCIMB;
b. a strain comprising a 16S-rRNA sequence comprising the nucleic acid sequence set forth in SEQ ID NO:9;
c. a strain comprising at least one genomic marker comprising the nucleic acid sequence set forth in any one of SEQ ID NOs:48-50;
and any combination thereof,
(12) strain LAV1000965, the strain being selected from the group consisting of:
a. a strain deposited under Accession Number 43963 at NCIMB;
b. a strain comprising a 16S-rRNA sequence comprising the nucleic acid sequence set forth in SEQ ID NO:8;
c. a strain comprising at least one genomic marker comprising the nucleic acid sequence set forth in any one of SEQ ID NOs:43-47;
and any combination thereof,
(13) strain LAV1000933, the strain being selected from the group consisting of:
a. a strain comprising a 16S-rRNA sequence comprising the nucleic acid sequence set forth in SEQ ID NO:10;
b. a strain comprising at least one genomic marker comprising the nucleic acid sequence set forth in any one of SEQ ID NOs:51-55;
and a combination thereof,
(14) strain LAV104661, the strain being selected from the group consisting of
d. a strain comprising a 16S-rRNA sequence comprising the nucleic acid sequence set forth in SEQ ID NO:14;
e. a strain comprising at least one genomic marker comprising the nucleic acid sequence set forth in any one of SEQ ID NOs:67-68;
and any combination thereof,
(15) strain LAV104665, the strain being selected from the group consisting of
a. a strain comprising a 16S-rRNA sequence comprising the nucleic acid sequence set forth in SEQ ID NO:15;
b. a strain comprising at least one genomic marker comprising the nucleic acid sequence set forth in any one of SEQ ID NOs:69-71;
and a combination thereof,
(16) strain LAV104794, the strain being selected from the group consisting of
a. a strain comprising a 16S-rRNA sequence comprising the nucleic acid sequence set forth in SEQ ID NO:16;
b. a strain comprising at least one genomic marker comprising the nucleic acid sequence set forth in any one of SEQ ID NOs:72-74;
and a combination thereof,
(17) strain LAV104618, the strain being selected from the group consisting of
a. a strain comprising a 16S-rRNA sequence comprising the nucleic acid sequence set forth in SEQ ID NO:17;
b. a strain comprising at least one genomic marker comprising the nucleic acid sequence set forth in any one of SEQ ID NOs:75-77;
and a combination thereof,
(18) strain LAV104692, the strain being selected from the group consisting of
a. a strain comprising a 16S-rRNA sequence comprising the nucleic acid sequence set forth in SEQ ID NO:18;
b. a strain comprising at least one genomic marker comprising the nucleic acid sequence set forth in any one of SEQ ID NOs:78-80;
and a combination thereof,
(19) strain LAV104891, the strain being selected from the group consisting of
a. a strain comprising a 16S-rRNA sequence comprising the nucleic acid sequence set forth in SEQ ID NO:19;
b. a strain comprising at least one genomic marker comprising the nucleic acid sequence set forth in any one of SEQ ID NOs:81-83;
and a combination thereof,
(20) strain LAV104630, the strain being selected from the group consisting of
a. a strain comprising a 16S-rRNA sequence comprising the nucleic acid sequence set forth in SEQ ID NO:20;
b. a strain comprising at least one genomic marker comprising the nucleic acid sequence set forth in any one of SEQ ID NOs:84-85;
and a combination thereof,
(21) strain LAV104664, the strain being selected from the group consisting of
a. a strain comprising a 16S-rRNA sequence comprising the nucleic acid sequence set forth in SEQ ID NO:21;
b. a strain comprising at least one genomic marker comprising the nucleic acid sequence set forth in any one of SEQ ID NOs:86-88;
and a combination thereof,
(22) strain LAV104922, the strain being selected from the group consisting of
a. a strain comprising a 16S-rRNA sequence comprising the nucleic acid sequence set forth in SEQ ID NO:22;
b. a strain comprising at least one genomic marker comprising the nucleic acid sequence set forth in any one of SEQ ID NOs:89-91;
and a combination thereof,
(23) strain LAV59524, the strain being selected from the group consisting of
a. a strain comprising a 16S-rRNA sequence comprising the nucleic acid sequence set forth in SEQ ID NO:23;
b. a strain comprising at least one genomic marker comprising the nucleic acid sequence set forth in any one of SEQ ID NOs:92-94;
and a combination thereof,
(24) strain LAV104961, the strain being selected from the group consisting of
a. a strain comprising a 16S-rRNA sequence comprising the nucleic acid sequence set forth in SEQ ID NO:25;
b. a strain comprising at least one genomic marker comprising the nucleic acid sequence set forth in any one of SEQ ID NOs:95-97;
and a combination thereof,
(25) strain LAV104960, the strain comprising a 16S-rRNA sequence comprising the nucleic acid sequence set forth in SEQ ID NO:24; and
(26) strain LAV104533, the strain comprising a 16S-rRNA sequence comprising the nucleic acid sequence set forth in SEQ ID NO:26.

2. The bacterial preparation according to claim 1, wherein strain LAV58567 is of the genus Lysobacter; strain LAV104662 is of the genus Sphingobium; strains LAV104858, LAV1000636, LAV1001107, LAV1000847, LAV1000965, LAV104665, LAV104794, and LAV104664, each is of the genus Pseudomonas; strains LAV104632 and LAV59524, each is of the genus Bacillus; strains LAV104629 and LAV104692, each is of the genus Pantoea; strain LAV1000506 is of the genus Gordonia; strain LAV1005101 is of the genus Pseudoxanthomonas; strain LAV1001081 is of the genus Rahnella; strain LAV1000933 is of the genus Paenibacillus; strain LAV104661 is of the genus Erwinia; strain LAV104618 is of the genus Ensifer; strains LAV104891 and LAV104922, each is of the genus Acinetobacter; strain LAV105630 is of the genus Enterobacter; strains LAV104960 and LAV104961, each is of the genus Streptomyces; and strain LAV104533 is of the genus Actinokineospora.

3. The bacterial preparation according to claim 1, wherein:
the functional homolog of bacterial strain LAV58567 comprises a 16S-rRNA sequence at least 97% identical to SEQ ID NO:5; at least one genomic marker having at least 95% local identity to a nucleic acid sequence set forth in any one of SEQ ID NOs:36-38 over 90% coverage; or a combination thereof;
the functional homolog of bacterial strain LAV104662 comprises a 16S-rRNA sequence at least 97% identical to SEQ ID NO:4; at least one genomic marker having at least 95% local identity to a nucleic acid sequence set forth in any one of SEQ ID NOs:33-35 over 90% coverage; or a combination thereof;
the functional homolog of bacterial strain LAV104858 comprises a 16S-rRNA sequence at least 97.9% identical to SEQ ID NO:3; at least one genomic marker having at least 95% local identity to a nucleic acid sequence set forth in any one of SEQ ID NOs:30-32 over 90% coverage; or a combination thereof;
the functional homolog of bacterial strain LAV104632 comprises a 16S-rRNA sequence at least 97% identical to SEQ ID NO:2; at least one genomic marker having at least 95% local identity to a nucleic acid sequence set forth in SEQ ID NO:29 over 90% coverage; or a combination thereof;
the functional homolog of bacterial strain LAV104629 comprises a 16S-rRNA sequence at least 97% identical to SEQ ID NO:1; at least one genomic marker having at least 95% local identity to a nucleic acid sequence set forth in any one of SEQ ID NOs:27-28 over 90% coverage; or a combination thereof;
the functional homolog of bacterial strain LAV1000636 comprises a 16S-rRNA sequence at least 98.7% identical to SEQ ID NO:6; at least one genomic marker having at least 95% local identity to a nucleic acid sequence set forth in SEQ ID NO:39 over 90% coverage, or a combination thereof;
the functional homolog of bacterial strain LAV1000506 comprises a 16S-rRNA sequence at least 97% identical to SEQ ID NO:7; at least one genomic marker having at least 95% local identity to a nucleic acid sequence set forth in any one of SEQ ID NOs:40-42 over 90% coverage; or a combination thereof;
the functional homolog of bacterial strain LAV1005101 comprises a 16S-rRNA sequence at least 97% identical to SEQ ID NO:13; at least one genomic marker having at least 95% local identity to a nucleic acid sequence set forth in any one of SEQ ID NOs:62-66 over 90% coverage; or a combination thereof;
the functional homolog of bacterial strain LAV10011071 comprises a 16S-rRNA sequence at least 97% identical to SEQ ID NO:12; at least one genomic marker having at least 95% local identity to a nucleic acid sequence set forth in any one of SEQ ID NOs:57-61 over 90% coverage; or a combination thereof;
the functional homolog of bacterial strain LAV1001081 comprises a 16S-rRNA sequence at least 97% identical to SEQ ID NO:11; a genomic marker having at least 95% local identity to the nucleic acid sequence set forth in SEQ ID NO:56 over 90% coverage; or a combination thereof;
the functional homolog of bacterial strain LAV1000847 comprises a 16S-rRNA sequence at least 99.5% identical to SEQ ID NO:9; at least one genomic marker having at least 95% local identity to a nucleic acid sequence set forth in any one of SEQ ID NOs:48-50 over 90% coverage; or a combination thereof;
the functional homolog of bacterial strain LAV1000965 comprises a 16S-rRNA sequence at least 97.6% identical to SEQ ID NO:8; at least one genomic marker having at least 95% local identity to a nucleic acid sequence set forth in any one of SEQ ID NOs:43-47 over 90% coverage; or a combination thereof;
the functional homolog of bacterial strain LAV1000933 comprises a 16S-rRNA sequence at least 97% identical to SEQ ID NO:10; at least one genomic marker having at least 95% local identity to a nucleic acid sequence set forth in any one of SEQ ID NOs:51-55 over 90% coverage; or a combination thereof;
the functional homolog of bacterial strain LAV104661 comprises a 16S-rRNA sequence at least 97% identical to SEQ ID NO:14; at least one genomic marker having at least 95% local identity to a nucleic acid sequence set forth in any one of SEQ ID NOs:67-68 over 90% coverage; or a combination thereof;
the functional homolog of bacterial strain LAV104665 comprises a 16S-rRNA sequence at least 97% identical to SEQ ID NO:15; at least one genomic marker having at least 95% local identity to a nucleic acid sequence set forth in any one of SEQ ID NOs:69-71 over 90% coverage; or a combination thereof;
the functional homolog of bacterial strain LAV104794 comprises a 16S-rRNA sequence at least 97% identical to SEQ ID NO:16; at least one genomic marker having at least 95% local identity to a nucleic acid sequence set forth in any one of SEQ ID NOs:72-74 over 90% coverage; or a combination thereof;
the functional homolog of bacterial strain LAV104618 comprises a 16S-rRNA sequence at least 97.1% identical to SEQ ID NO:17; at least one genomic marker having at least 95% local identity to a nucleic acid sequence set forth in any one of SEQ ID NOs:75-77 over 90% coverage, or a combination thereof;
the functional homolog of bacterial strain LAV104692 comprises a 16S-rRNA sequence at least 97% identical to SEQ ID NO:18; at least one genomic marker having at least 95% local identity to a nucleic acid sequence set forth in any one of SEQ ID NOs:78-80 over 90% coverage; or a combination thereof;
the functional homolog of bacterial strain LAV104891 comprises a 16S-rRNA sequence at least 97% identical to SEQ ID NO:19; at least one genomic marker having at least 95% local identity to a nucleic acid sequence set forth in any one of SEQ ID NOs:81-83 over 90% coverage; or a combination thereof;
the functional homolog of bacterial strain LAV104630 comprises a 16S-rRNA sequence at least 97% identical to SEQ ID NO:20; at least one genomic marker having at least 95% local identity to a nucleic acid sequence set forth in any one of SEQ ID NOs:84-85 over 90% coverage; or a combination thereof;
the functional homolog of bacterial strain LAV104664 comprises a 16S-rRNA sequence at least 97% identical to SEQ ID NO:21; at least one genomic marker having at least 95% local identity to a nucleic acid sequence set forth in any one of SEQ ID NOs:86-88 over 90% coverage; or a combination thereof;
the functional homolog of bacterial strain LAV104922 comprises a 16S-rRNA sequence at least 97% identical to SEQ ID NO:22; at least one genomic marker having at least 95% local identity to a nucleic acid sequence set forth in any one of SEQ ID NOs:89-91 over 90% coverage; or a combination thereof;
the functional homolog of bacterial strain LAV59524 comprises a 16S-rRNA sequence at least 97% identical to SEQ ID NO:23; at least one genomic marker having at least 95% local identity to a nucleic acid sequence set forth in any one of SEQ ID NOs:92-94 over 90% coverage; or a combination thereof;
the functional homolog of bacterial strain LAV104961 comprises a 16S-rRNA sequence at least 97% identical to SEQ ID NO:25; at least one genomic marker having at least 95% local identity to a nucleic acid sequence set forth in any one of SEQ ID NOs:95-97 over 90% coverage; or a combination thereof;
the functional homolog of bacterial strain LAV104960 comprises a 16S-rRNA sequence at least 97.9% identical to SEQ ID NO:24; and
the functional homolog of bacterial strain LAV104533 comprises a 16S-rRNA sequence at least 97% identical to SEQ ID NO:26.

4. The bacterial preparation according to claim 1, wherein the bacterial strain or functional homolog is characterized by a capability to inhibit the growth and/or the development and/or the activity of at least one plant pathogenic fungus and/or oomycete, and/or by a capability to protect a plant from the at least one pathogenic fungus and/or oomycete.

5-9. (canceled)

10. A lysate of the bacterial preparation according to claim 1, wherein the lysate is selected from the group consisting of a whole cell lysate of the at least one bacterial strain or functional homolog thereof, and a soluble fraction of the at least one bacterial strain or functional homolog thereof.

11. (canceled)

12. An extract of the bacterial preparation according to claim 1.

13. A whole cell broth collected from fermentation of the bacterial preparation according to claim 1.

14. An agricultural composition comprising the bacterial preparation according to claim 1, a lysate, an extract, or a whole cell broth thereof, further comprising an agriculturally acceptable diluent or carrier.

15. (canceled)

16. The agricultural composition according to claim 14, wherein the carrier is a plant seed.

17-18. (canceled)

19. A seed coated with the agricultural composition according to claim 14.

20-21. (canceled)

22. A method for at least one of enhancing and/or conferring resistance to a plant or a part thereof toward a disease caused by a phytopathogenic fungus and/or oomycete, and preventing or treating a plant disease caused by the phytopathogenic fungus and/or oomycete, comprising contacting the plant, a part thereof or the habitat of said plant with at least one bacterial strain or a functional homolog thereof, a bacterial preparation comprising same, a lysate thereof, an extract thereof, a whole cell broth thereof, or an agricultural composition comprising same, wherein the bacterial strain is selected from the group consisting of:
(1) strain LAV58567, the strain being selected from the group consisting of
a. a strain deposited under Accession Number 43954 at NCIMB;
b. a strain comprising a 16S-rRNA sequence comprising the nucleic acid sequence set forth in SEQ ID NO:5;
c. a strain comprising at least one genomic marker comprising the nucleic acid sequence set forth in any one of SEQ ID NOs:36-38:
and any combination thereof;
(2) strain LAV104662, the strain being selected from the group consisting of
a. a strain deposited under Accession Number 43955 at NCIMB;
b. a strain comprising a 16S-rRNA sequence comprising the nucleic acid sequence set forth in SEQ ID NO:4;
c. a strain comprising at least one genomic marker comprising the nucleic acid sequence set forth in any one of SEQ ID NOs:33-35:
and any combination thereof;
(3) strain LAV104858, the strain being selected from the group consisting of
a. a strain deposited under Accession Number 43956 at NCIMB;
b. a strain comprising a 16S-rRNA sequence comprising the nucleic acid sequence set forth in SEQ ID NO:3;
c. a strain comprising at least one genomic marker comprising the nucleic acid sequence set forth in any one of SEQ ID NOs:30-32:
and any combination thereof;
(4) strain LAV104632, the strain being selected from the group consisting of
a. a strain deposited under Accession Number 43957 at NCIMB;
b. a strain comprising a 16S-rRNA sequence comprising the nucleic acid sequence set forth in SEQ ID NO:2;
c. a strain comprising at least one genomic marker comprising the nucleic acid sequence set forth in SEQ ID NO:29:
and any combination thereof;
(5) strain LAV104629, the strain being selected from the group consisting of
a. a strain deposited under Accession Number 43958 at NCIMB;
b. a strain comprising a 16S-rRNA sequence comprising the nucleic acid sequence set forth in SEQ ID NO:1;
c. a strain comprising at least one genomic marker comprising the nucleic acid sequence set forth in any one of SEQ ID NOs:27-28:
and any combination thereof;
(6) strain LAV1000636, the strain being selected from the group consisting of
a. a strain deposited under Accession Number 43964 at NCIMB;
b. a strain comprising a 16S-rRNA sequence comprising the nucleic acid sequence set forth in SEQ ID NO:6;
c. a strain comprising at least one genomic marker comprising the nucleic acid sequence set forth in SEQ ID NO:39:
and any combination thereof;
(7) strain LAV1000506, the strain being selected from the group consisting of
a. a strain deposited under Accession Number 43965 at NCIMB;
b. a strain comprising a 16S-rRNA sequence comprising the nucleic acid sequence set forth in SEQ ID NO:7;
c. a strain comprising at least one genomic marker comprising the nucleic acid sequence set forth in any one of SEQ ID NOs:40-42:
and any combination thereof;
(8) strain LAV1005101, the strain being selected from the group consisting of:
a. a strain deposited under Accession Number 43959 at NCIMB;
b. a strain comprising a 16S-rRNA sequence comprising the nucleic acid sequence set forth in SEQ ID NO:13;
c. a strain comprising at least one genomic marker comprising the nucleic acid sequence set forth in any one of SEQ ID NOs:62-66:
and any combination thereof;
(9) strain LAV1001107, the strain being selected from the group consisting of:
a. a strain deposited under Accession Number 43960 at NCIMB;
b. a strain comprising a 16S-rRNA sequence comprising the nucleic acid sequence set forth in SEQ ID NO:12;
c. a strain comprising at least one genomic marker comprising the nucleic acid sequence set forth in any one of SEQ ID NOs:57-61:
and any combination thereof;
(10) strain LAV1001081, the strain being selected from the group consisting of:
a. a strain deposited under Accession Number 43961 at NCIMB;
b. a strain comprising a 16S-rRNA sequence comprising the nucleic acid sequence set forth in SEQ ID NO:11;
c. a strain comprising at least one genomic marker comprising the nucleic acid sequence set forth in SEQ ID NO:56:
and any combination thereof;
(11) strain LAV1000847, the strain being selected from the group consisting of:
a. a strain deposited under Accession Number 43962 at NCIMB;
b. a strain comprising a 16S-rRNA sequence comprising the nucleic acid sequence set forth in SEQ ID NO:9;
c. a strain comprising at least one genomic marker comprising the nucleic acid sequence set forth in any one of SEQ ID NOs:48-50:
and any combination thereof;
(12) strain LAV1000965, the strain being selected from the group consisting of:
a. a strain deposited under Accession Number 43963 at NCIMB;
b. a strain comprising a 16S-rRNA sequence comprising the nucleic acid sequence set forth in SEQ ID NO:8;
c. a strain comprising at least one genomic marker comprising the nucleic acid sequence set forth in any one of SEQ ID NOs:43-47;
and any combination thereof;
(13) strain LAV1000933, the strain being selected from the group consisting of:
a. a strain comprising a 16S-rRNA sequence comprising the nucleic acid sequence set forth in SEQ ID NO:10;
b. a strain comprising at least one genomic marker comprising the nucleic acid sequence set forth in any one of SEQ ID NOs:51-55:
and a combination thereof;
(14) strain LAV104661, the strain being selected from the group consisting of
a. a strain comprising a 16S-rRNA sequence comprising the nucleic acid sequence set forth in SEQ ID NO:14;
b. a strain comprising at least one genomic marker comprising the nucleic acid sequence set forth in any one of SEQ ID NOs:67-68:
and any combination thereof;
(15) strain LAV104665, the strain being selected from the group consisting of
a. a strain comprising a 16S-rRNA sequence comprising the nucleic acid sequence set forth in SEQ ID NO:15;
b. a strain comprising at least one genomic marker comprising the nucleic acid sequence set forth in any one of SEQ ID NOs:69-71:
and a combination thereof;
(16) strain LAV104794, the strain being selected from the group consisting of
a. a strain comprising a 16S-rRNA sequence comprising the nucleic acid sequence set forth in SEQ ID NO:16;
b. a strain comprising at least one genomic marker comprising the nucleic acid sequence set forth in any one of SEQ ID NOs:72-74:
and a combination thereof;
(17) strain LAV104618, the strain being selected from the group consisting of
a. a strain comprising a 16S-rRNA sequence comprising the nucleic acid sequence set forth in SEQ ID NO:17;
b. a strain comprising at least one genomic marker comprising the nucleic acid sequence set forth in any one of SEQ ID NOs:75-77:
and a combination thereof;
(18) strain LAV104692, the strain being selected from the group consisting of
a. a strain comprising a 16S-rRNA sequence comprising the nucleic acid sequence set forth in SEQ ID NO:18;
b. a strain comprising at least one genomic marker comprising the nucleic acid sequence set forth in any one of SEQ ID NOs:78-80:
and a combination thereof;
(19) strain LAV104891, the strain being selected from the group consisting of
a. a strain comprising a 16S-rRNA sequence comprising the nucleic acid sequence set forth in SEQ ID NO:19;
b. a strain comprising at least one genomic marker comprising the nucleic acid sequence set forth in any one of SEQ ID NOs:81-83:
and a combination thereof;
(20) strain LAV104630, the strain being selected from the group consisting of
a. a strain comprising a 16S-rRNA sequence comprising the nucleic acid sequence set forth in SEQ ID NO:20;
b. a strain comprising at least one genomic marker comprising the nucleic acid sequence set forth in any one of SEQ ID NOs:84-85:
and a combination thereof;
(21) strain LAV104664, the strain being selected from the group consisting of
a. a strain comprising a 16S-rRNA sequence comprising the nucleic acid sequence set forth in SEQ ID NO:21;
b. a strain comprising at least one genomic marker comprising the nucleic acid sequence set forth in any one of SEQ ID NOs:86-88:
and a combination thereof;
(22) strain LAV104922, the strain being selected from the group consisting of
a. a strain comprising a 16S-rRNA sequence comprising the nucleic acid sequence set forth in SEQ ID NO:22;
b. a strain comprising at least one genomic marker comprising the nucleic acid sequence set forth in any one of SEQ ID NOs:89-91:
and a combination thereof;
(23) strain LAV59524, the strain being selected from the group consisting of
a. a strain comprising a 16S-rRNA sequence comprising the nucleic acid sequence set forth in SEQ ID NO:23;
b. a strain comprising at least one genomic marker comprising the nucleic acid sequence set forth in any one of SEQ ID NOs:92-94:
and a combination thereof;
(24) strain LAV104961, the strain being selected from the group consisting of
a. a strain comprising a 16S-rRNA sequence comprising the nucleic acid sequence set forth in SEQ ID NO:25;
b. a strain comprising at least one genomic marker comprising the nucleic acid sequence set forth in any one of SEQ ID NOs:95-97:
and a combination thereof;
(25) strain LAV104960, the strain comprising a 16S-rRNA sequence comprising the nucleic acid sequence set forth in SEQ ID NO:24; and
(26) strain LAV104533, the strain comprising a 16S-rRNA sequence comprising the nucleic acid sequence set forth in SEQ ID NO:26.

23-31. (canceled)

32. The method of 22, wherein the phytopathogenic fungus is of a genus selected from the group consisting of Fusarium, Botrytis, Erysiphe, Aspergillus, and Rhizopus and wherein the oomycete is of a genus selected from the group consisting of Plasmopara, Pythium, Phytophthora, and Pseudoperonospora.

33-35. (canceled)

36. The method of claim 32, wherein the pathogenic fungus is Fusarium graminearum, and the bacterial strain is selected from the group consisting of LAV58567, LAV104794, LAV104632, LAV104664, LAV104630, LAV104891 LAV104922, LAV59524, LAV1000965, LAV1000847, LAV1000933, LAV1001081, LAV1001107, and LAV1005101, functional homologs thereof and any combination thereof.

37. The method of claim 32, wherein the pathogenic fungus is Fusarium verticillioides, and the bacterial strain is selected from the group consisting of LAV58567, LAV104794, LAV104661, LAV104662, LAV104629, LAV104665, LAV104664, LAV104692, LAV104630, LAV104618, LAV104891, LAV104922 LAV59524, LAV104960, LAV104961, LAV1000965, LAV1000847, LAV1000933, LAV1001081, LAV1001107, and LAV1005101, functional homologs thereof and any combination thereof.

38. The method of claim 32, wherein the pathogenic fungus is Botrytis cinerea, and the bacterial strain is selected from the group consisting of LAV104794, LAV104661, LAV104662, LAV104632, LAV104665, LAV104858, LAV104692, LAV104630, LAV104618, LAV104891, LAV104922, LAV59524 LAV104960, LAV104961, LAV1000965, LAV1000847, LAV1000933, LAV1001081, LAV1001107, and LAV1005101, functional homologs thereof and any combination thereof.

39. The method of claim 32, wherein the pathogenic oomycete is Pythium aphanidermatum, and the bacterial strain is selected from the group consisting of LAV104794, LAV104629, LAV104665, LAV104858, LAV104664, LAV104692, LAV104630, LAV104891, LAV104922, LAV59524, LAV104961, LAV58567, LAV104661, LAV104662, LAV104632 LAV104618, LAV1000965, LAV1000847, LAV1000933, LAV1001081, LAV1001107, and LAV1005101, functional homologs thereof and any combination thereof.

40. The method of claim 32, wherein the pathogenic oomycete is Pythium irregulare, and the bacterial strain is selected from the group consisting of LAV104661, LAV104662, LAV104632, LAV104629, LAV104665, LAV104664, LAV104630, LAV104618, LAV104618, LAV1000506, LAV1000636, LAV1000847, LAV1000933, LAV1001081, LAV1001107, and LAV1005101, functional homologs thereof and any combination thereof.

41. The method of claim 32, wherein the pathogenic oomycete is Plasmopara viticola, and the bacterial strain is selected from the group consisting of LAV59524, LAV104960, LAV104961, LAV104533, and LAV58567, functional homologs thereof and any combination thereof.

42-43. (canceled)

44. A method of obtaining a modified bacterial strain having an improved capability to confer or enhance the resistance of a plant towards pathogenic fungi and/or oomycetes, or to protect the plant from a disease caused by the pathogenic fungi and/or oomycetes, as compared to a parent unmodified bacterial strain, comprising:
(a) culturing a parent bacterial strain having a capability to confer or enhance the resistance of a plant towards pathogenic fungi and/or oomycetes, or to protect the plant from a disease caused by the pathogenic fungi and/or oomycetes, under conditions suitable for expanding a population of the bacterial strain and allowing development of at least one bacterial mutant; and
(b) selecting at least one bacterial mutant resultant of step (a) for an improved capability to confer or enhance the resistance of a plant towards the pathogenic fungi and/or oomycetes, or to protect the plant from a disease caused by said pathogenic fungi and/or oomycetes,
thereby obtaining the modified bacterial strain having the improved capability to confer or enhance the resistance of a plant towards said pathogenic fungi and/or oomycetes, or to protect the plant from a disease caused by said pathogenic fungi and/or oomycetes as compared to the unmodified parent bacterial strain.

45-46. (canceled)

47. The method of claim 22, wherein,
the functional homolog of bacterial strain LAV58567 comprises a 16S-rRNA sequence at least 97% identical to SEQ ID NO:5; at least one genomic marker having at least 95% local identity to a nucleic acid sequence set forth in any one of SEQ ID NOs:36-38 over 90% coverage; or a combination thereof;
the functional homolog of bacterial strain LAV104662 comprises a 16S-rRNA sequence at least 97% identical to SEQ ID NO:4; at least one genomic marker having at least 95% local identity to a nucleic acid sequence set forth in any one of SEQ ID NOs:33-35 over 90% coverage; or a combination thereof;
the functional homolog of bacterial strain LAV104858 comprises a 16S-rRNA sequence at least 97.9% identical to SEQ ID NO:3; at least one genomic marker having at least 95% local identity to a nucleic acid sequence set forth in any one of SEQ ID NOs:30-32 over 90% coverage; or a combination thereof;
the functional homolog of bacterial strain LAV104632 comprises a 16S-rRNA sequence at least 97% identical to SEQ ID NO:2; at least one genomic marker having at least 95% local identity to a nucleic acid sequence set forth in SEQ ID NO:29 over 90% coverage; or a combination thereof;
the functional homolog of bacterial strain LAV104629 comprises a 16S-rRNA sequence at least 97% identical to SEQ ID NO:1; at least one genomic marker having at least 95% local identity to a nucleic acid sequence set forth in any one of SEQ ID NOs:27-28 over 90% coverage; or a combination thereof;
the functional homolog of bacterial strain LAV1000636 comprises a 16S-rRNA sequence at least 98.7% identical to SEQ ID NO:6; at least one genomic marker having at least 95% local identity to a nucleic acid sequence set forth in SEQ ID NO:39 over 90% coverage, or a combination thereof;
the functional homolog of bacterial strain LAV1000506 comprises a 16S-rRNA sequence at least 97% identical to SEQ ID NO:7; at least one genomic marker having at least 95% local identity to a nucleic acid sequence set forth in any one of SEQ ID NOs:40-42 over 90% coverage; or a combination thereof;
the functional homolog of bacterial strain LAV1005101 comprises a 16S-rRNA sequence at least 97% identical to SEQ ID NO:13; at least one genomic marker having at least 95% local identity to a nucleic acid sequence set forth in any one of SEQ ID NOs:62-66 over 90% coverage; or a combination thereof;
the functional homolog of bacterial strain LAV10011071 comprises a 16S-rRNA sequence at least 97% identical to SEQ ID NO:12; at least one genomic marker having at least 95% local identity to a nucleic acid sequence set forth in any one of SEQ ID NOs:57-61 over 90% coverage; or a combination thereof;
the functional homolog of bacterial strain LAV1001081 comprises a 16S-rRNA sequence at least 97% identical to SEQ ID NO:11; a genomic marker having at least 95% local identity to the nucleic acid sequence set forth in SEQ ID NO:56 over 90% coverage; or a combination thereof;
the functional homolog of bacterial strain LAV1000847 comprises a 16S-rRNA sequence at least 99.5% identical to SEQ ID NO:9; at least one genomic marker having at least 95% local identity to a nucleic acid sequence set forth in any one of SEQ ID NOs:48-50 over 90% coverage; or a combination thereof;
the functional homolog of bacterial strain LAV1000965 comprises a 16S-rRNA sequence at least 97.6% identical to SEQ ID NO:8; at least one genomic marker having at least 95% local identity to a nucleic acid sequence set forth in any one of SEQ ID NOs:43-47 over 90% coverage; or a combination thereof;
the functional homolog of bacterial strain LAV1000933 comprises a 16S-rRNA sequence at least 97% identical to SEQ ID NO:10; at least one genomic marker having at least 95% local identity to a nucleic acid sequence set forth in any one of SEQ ID NOs:51-55 over 90% coverage; or a combination thereof;
the functional homolog of bacterial strain LAV104661 comprises a 16S-rRNA sequence at least 97% identical to SEQ ID NO:14; at least one genomic marker having at least 95% local identity to a nucleic acid sequence set forth in any one of SEQ ID NOs:67-68 over 90% coverage; or a combination thereof;
the functional homolog of bacterial strain LAV104665 comprises a 16S-rRNA sequence at least 97% identical to SEQ ID NO:15; at least one genomic marker having at least 95% local identity to a nucleic acid sequence set forth in any one of SEQ ID NOs:69-71 over 90% coverage; or a combination thereof;
the functional homolog of bacterial strain LAV104794 comprises a 16S-rRNA sequence at least 97% identical to SEQ ID NO:16; at least one genomic marker having at least 95% local identity to a nucleic acid sequence set forth in any one of SEQ ID NOs:72-74 over 90% coverage; or a combination thereof;
the functional homolog of bacterial strain LAV104618 comprises a 16S-rRNA sequence at least 97.1% identical to SEQ ID NO:17; at least one genomic marker having at least 95% local identity to a nucleic acid sequence set forth in any one of SEQ ID NOs:75-77 over 90% coverage; or a combination thereof;
the functional homolog of bacterial strain LAV104692 comprises a 16S-rRNA sequence at least 97% identical to SEQ ID NO:18; at least one genomic marker having at least 95% local identity to a nucleic acid sequence set forth in any one of SEQ ID NOs:78-80 over 90% coverage; or a combination thereof;
the functional homolog of bacterial strain LAV104891 comprises a 16S-rRNA sequence at least 97% identical to SEQ ID NO:19; at least one genomic marker having at least 95% local identity to a nucleic acid sequence set forth in any one of SEQ ID NOs:81-83 over 90% coverage; or a combination thereof;
the functional homolog of bacterial strain LAV104630 comprises a 16S-rRNA sequence at least 97% identical to SEQ ID NO:20; at least one genomic marker having at least 95% local identity to a nucleic acid sequence set forth in any one of SEQ ID NOs:84-85 over 90% coverage; or a combination thereof;
the functional homolog of bacterial strain LAV104664 comprises a 16S-rRNA sequence at least 97% identical to SEQ ID NO:21; at least one genomic marker having at least 95% local identity to a nucleic acid sequence set forth in any one of SEQ ID NOs:86-88 over 90% coverage; or a combination thereof;
the functional homolog of bacterial strain LAV104922 comprises a 16S-rRNA sequence at least 97% identical to SEQ ID NO:22; at least one genomic marker having at least 95% local identity to a nucleic acid sequence set forth in any one of SEQ ID NOs:89-91 over 90% coverage; or a combination thereof;
the functional homolog of bacterial strain LAV59524 comprises a 16S-rRNA sequence at least 97% identical to SEQ ID NO:23; at least one genomic marker having at least 95% local identity to a nucleic acid sequence set forth in any one of SEQ ID NOs:92-94 over 90% coverage; or a combination thereof;
the functional homolog of bacterial strain LAV104961 comprises a 16S-rRNA sequence at least 97% identical to SEQ ID NO:25; at least one genomic marker having at least 95% local identity to a nucleic acid sequence set forth in any one of SEQ ID NOs:95-97 over 90% coverage; or a combination thereof;
the functional homolog of bacterial strain LAV104960 comprises a 16S-rRNA sequence at least 97.9% identical to SEQ ID NO:24; and
the functional homolog of bacterial strain LAV104533 comprises a 16S-rRNA sequence at least 97% identical to SEQ ID NO:26.
